(19) United States
(12) Patent Application Publication
(10) Pub. No.: US 2016/0090638 A1
(43) Pub. Date: Mar. 31, 2016

(54) METHODS OF PROGNOSTICALLY CLASSIFYING AND TREATING GLANDULAR CANCERS

(71) Applicants: Winston Chung-Yuan YU, Santa Monica, CA (US); NATIONAL HEALTH RESEARCH INSTITUTES, Zhunan Town (TW)

(72) Inventors: Kun-Chih Kelvin TSAI, Zhunan Town (TW); Chi-Rong LI, Taipei (TW); Chung-Chi HSU, Zhunan Town (TW)

(73) Assignee: NATIONAL HEALTH RESEARCH INSTITUTES, Zhunan Township (TW)

(21) Appl. No.: 14/891,959

(22) PCT Filed: May 16, 2014
(86) PCT No.: PCT/US14/38504

§ 371 Date: Nov. 17, 2015

Related U.S. Application Data
(60) Provisional application No. 61/824,679, filed on May 17, 2013.

(51) Int. Cl. C12Q 1/68; G01N 33/574

(52) U.S. Cl. CPC C12Q 1/6886; G01N 33/57438; C12Q 2600/118; C12Q 2600/112; C12Q 2600/158; G01N 2800/52; G01N 2800/56; G01N 2333/4703

(57) ABSTRACT

This disclosure includes the identification of molecular markers, including ASPM, ATP9A, ACOX3, CD-C45L, SLC40A1, AGR2, and those found in TABLE 2, that are associated with the differentiation and the clinical prognosis of pancreatic cancer. More specifically, the disclosure includes the identification of sets of gene markers whose expression levels can be used to distinguish pancreatic cancers with higher degrees of differentiation from those with lower degrees of differentiation. These markers can be used to predict clinical prognosis of pancreatic cancer, including disease progression, recurrence or death of the hosts. The disclosure also provides methods of treating glandular cancers and kits for assaying glandular cancers, such as pancreatic cancer, breast cancer, and prostate cancer, by inhibiting the expression of ASPM or its ability to activate or maintain the Wnt signaling activity and/or the cancer stem cell populations of said glandular cancers.

### CROSS-REFERENCE TO RELATED APPLICATIONS

This application claims the benefit of U.S. Provisional Application No. 61/824,679, filed May 17, 2013, herein incorporated by reference.

### FIELD OF THE INVENTION

This disclosure includes systems and methods kits for classifying pancreatic cancer and glandular cancers and predicting disease progression, recurrence, and death. This disclosure also includes methods and kits for treatment of pancreatic cancer and glandular cancers.

### BACKGROUND

Pancreatic ductal adenocarcinoma (PDAC) is a devastating malignancy. Because of the paucity of symptoms in early diseases and the aggressive behaviors of the tumors, less than 20% of patients with PDAC present with localized and resectable diseases at the time of diagnosis. Even with curative-intent surgery, the majority of patients with initially localized tumors developed recurrent or metastatic diseases and only a small subset (18-26%) of the patients could attain long-term survival (Ahmad et al., 2001; Oettle et al., 2007). Thus, further improvements in the prognosis of patients with localized PDAC may rely on elucidating the pathogenesis underlying tumor recurrence and clinically reliable prognostic prediction that may guide patient-tailored treatment plans.

The malignant transformation of glandular epithelium involves a gradual and variable loss of the normal glandular architectures. As such human pancreatic cancer frequently displays considerable intra-tumoral heterogeneity in glandular differentiation, a factor widely used for the pathological classification of glandular cancer such as the Gleason grading system in prostate cancer (Gleason, 1992). Thus, glandular differentiation has been used in the histopathological assessment for other types of gland-derived malignancies, including breast cancer and pancreatic cancer (Adsay et al., 2005; Gleason, 1992; Hruban and Fukushima, 2007; Rakha et al., 2008). However, morphology-based pathological classification systems are only modestly prognostic and do not allow for risk stratification of pancreatic cancer with similar histopathological characteristics. Assessments of tissue architectures did not provide functional or mechanistic insights into observed tumor variations. There is thus a critical need for pathway-informed and molecularly-based diagnostic assays with increased accuracy in the prediction of clinical outcome in pancreatic cancer.

Recently, high throughput genomic profiling techniques have facilitated the molecular characterization of human malignant tumors, including pancreatic cancer (Glinsky et al., 2004; Henshall et al., 2003; Singh et al., 2002; Stratford et al., 2010; van't Veer et al., 2002; van de Vijver et al., 2002). The profound prognostic utilities of these genomic markers point to the intrinsic molecular characteristic of tumors as a crucial determinant to their clinical behaviors (Ramaswamy et al., 2003). For instance, by comparing the gene expression patterns of metastatic and primary PDAC, Stratford and colleagues identified a 6-gene metastasis-associated signature that was predictive of survival in patients with early-stage PDAC (Stratford et al., 2010). By comparing the transcriptomes of 27 micro-dissected PDAC tissues, Collisson and colleagues identified a 62-gene signature termed “PDAssigner” that could be used to define molecular subtypes in PDAC (Collisson et al., 2011).

It should be noted that the above mentioned molecular patterns were identified from clinical pancreatic tumor specimen and might only reflect established tumor characteristics without providing mechanisms underlying the pathogenesis of these tumor variations. In this regard, knowledge-based approaches offer an opportunity to identify more rational markers or classification systems that benefit clinical decision-making and therapeutic advancement. Such approaches have been used to establish the prognostic roles of gene profiles associated with tumor progenitor cells, stromal activation or tissue differentiation in several types of solid tumors (Chang et al., 2004; Fournier et al., 2006; Liu et al., 2007; Sotiriou et al., 2006).

Currently prevailing models of tumorigenesis suggest that tissue differentiation and tumor progression share similar gene regulations and molecular pathways. Molecular changes associated with the differentiation process of glandular epithelium may be difficult to study in vivo. However, a physiological relevant three-dimensional organotypic culture model has been used to recapitulate the structural and functional differentiation processes of mammary acini, the basic structural unit of normal mammary epithelium (Debnath and Brugge, 2005; Lee et al., 2007). Similar models have successfully recapitulated the morphogenetic and differentiation processes of pancreatic, pancreatic and pulmonary epithelium (Gutierrez-Barrera et al., 2007; Mondrinos et al., 2006; Webber et al., 1997). Comparative gene expression analysis using this developmental model has led to the identification of gene expression profiles and marker genes that showed significant association with breast cancer prognosis (Fournier et al., 2006; Kenny et al., 2007). Whether or not the same paradigm can be applied to pancreatic cancer remains unclear.

The human ASPM gene encodes a large (409.8 kDa) and multi-functional protein that plays a critical role in neurogenesis, neuronal migration and the expansion of glioma stem cells (Bikeye et al., 2010; Buchman et al., 2011). ASPM was initially identified as a centrosomal protein that regulates neurogenesis and brain size (Bond et al., 2003; Kouprina et al., 2005). However, ASPM is now known to be widely expressed in a variety of fetal and adult tissues beyond the central nervous system (Bruning-Richardson et al., 2011; Kouprina et al., 2005). Recent studies also demonstrated that ASPM expression is up-regulated and prognostically important in several types of malignant tumors. For instance, ASPM expression positively correlated with the pathological grade of glioma and was up-regulated in recurrent tumors (Bikeye et al., 2010). ASPM expression also correlated with the pathological grade and poor survival in patients with ovarian cancer or hepatocellular carcinoma (Bikeye et al., 2010; Bruning-Richardson et al., 2011; Lin et al., 2008). Interestingly, these studies also demonstrated that ASPM was both cytoplasmic and nuclear localized in interphase and its cytoplasmic expression levels were highly variable among tumors (Bruning-Richardson et al., 2011), suggesting that it may have diverse biological functions in malignant tissues.

It is accordingly an object of certain embodiments to provide clinically reliable prognostic prediction that may guide patient-tailored treatment plans for individuals with pancreatic or glandular cancers. This is achieved by providing panels of diagnostic markers and methods of using them, as well as providing exemplary methods and kits for use in treating pancreatic or glandular cancers.

### SUMMARY

The current disclosure includes the identification of gene markers listed in TABLE 2, which are associated with the extent of differentiation in pancreatic cancer tissues, and the use of these markers to predict clinical prognosis of pancreatic cancer. More specifically, the disclosure includes the identification of sets of gene markers whose expression levels can be used to distinguish pancreatic cancers with higher degrees of differentiation from those with lower degrees of differentiation. In addition, the transcript or protein expression levels of these gene markers identified in the present disclosure can be used to predict clinical prognosis of pancreatic cancer, including disease progression, recurrence or death of subjects with pancreatic cancer. The present disclosure also includes the use of ASPM to predict clinical prognosis of other types of glandular cancers such as breast cancer, prostate cancer, colon cancer and gastric cancer. In addition, the present disclosure includes methods of treating glandular cancers, such as pancreatic cancer, breast cancer, and prostate cancer, by inhibiting the expression of ASPM or its ability to activate or maintain the Wnt signaling activity and/or the cancer stem cell populations of said glandular cancers.

In a specific embodiment of the above method, predicting clinical prognosis of pancreatic cancer comprises the determination of the transcript or protein expression levels of ASPM, ATP9A, ACOX3, CDC45L, SLC40A1, AGR2 and those found in TABLE 2, or any combination thereof in pancreatic tumor specimens obtained from biopsy or surgical procedures, and the use of combinations of the expression levels to forecast outcome of subjects carrying said pancreatic tumors.

In a specific embodiment, determining the transcript expression levels of said gene markers comprises polymerase chain reaction, northern blotting, RNase protection assay, or cDNA or oligonucleotide microarray analysis on frozen or formalin fixed paraffin embedded (FFPE) pancreatic tumor specimens.

In another embodiment, determining the protein expression levels of said gene markers comprises immunoblotting, immunohistochemistry, protein array or two-dimensional protein electrophoresis, or mass spectroscopy analysis.

In an embodiment of the above method, determining the protein expression levels comprises the use of antibodies specific to said markers and immunohistochemistry staining on frozen or FFPE pancreatic tumor tissues.

In another embodiment, the current disclosure describes the prediction of pancreatic cancer prognosis by determining the protein expression levels of ASPM, ATP9A, ACOX3, CDC45L, SLC40A1 or AGR2 or any combination thereof using specific antibodies and immunohistochemistry staining on frozen or FFPE pancreatic tumor specimens obtained from biopsy or surgical procedures.

The present disclosure also provides a kit for predicting the clinical prognosis of pancreatic cancer, comprising means for detecting in a tumor the transcript or the protein of ASPM, ATP9A, ACOX3, CDC45L, SLC40A1, AGR2, and those found in TABLE 2 or any combination of any of the foregoing.

In one embodiment, the kit for predicting the clinical prognosis of pancreatic cancer comprises specific antibodies for detecting in a tumor the protein of ASPM, ATP9A, ACOX3, CDC45L, SLC40A1 or AGR2 or any combination thereof.

The present disclosure additionally provides an array of nucleic acid probes specific for a transcript of ASPM, ATP9A, ACOX3, CDC45L, SLC40A1, AGR2, and those found in TABLE 2, or one or a plurality of housekeeping genes or any combination thereof for predicting the clinical prognosis of pancreatic cancer.

Another embodiment provides an array of antibodies or aptamers specific for a protein of ASPM, ATP9A, ACOX3, CDC45L, SLC40A1, AGR2, and those found in TABLE 2, or one or a plurality of housekeeping genes or any combination thereof for predicting the clinical prognosis of pancreatic cancer.

Certain embodiments relate to the use of ASPM to predict clinical prognosis of other types of glandular cancers such as breast cancer, prostate cancer, colon cancer and gastric cancer. In a specific embodiment, predicting clinical prognosis of glandular cancers comprises the determination of the transcript or protein expression levels of ASPM in tumor specimens obtained from biopsy or surgical procedures, and the use of combinations of said expression levels to forecast outcome of subjects carrying said glandular cancers. Certain embodiments relate to the prediction of glandular cancer prognosis by determining the protein expression levels of ASPM using specific antibodies and immunohistochemistry staining on frozen or FFPE tumor specimens obtained from biopsy or surgical procedures.

Certain embodiments provide methods of treating pancreatic cancer or other types of glandular cancers by inhibiting the expression and/or the activity of ASPM in said cancer. In one embodiment, these methods comprise the inhibition of ASPM expression by administering to an individual with said cancer a nucleic acid complimentary to an ASPM mRNA, including an siRNA, shRNA, microRNA, or antisense oligonucleotide.

In one embodiment, inhibiting the activity of ASPM comprises the administration of a nucleic acid complimentary to an ASPM mRNA, including an siRNA, shRNA, microRNA, or antisense oligonucleotide, that is sufficient to inhibit the ability of ASPM to activate the Wnt signaling pathway and/or cancer stem cell populations in said cancer.

In another embodiment, inhibiting the activity of ASPM comprises the administration of a nucleic acid complimentary to an ASPM mRNA, including an siRNA, shRNA, microRNA, or antisense oligonucleotide, that is sufficient to Inhibit the ability of ASPM to promote or to maintain cancer stem cell populations or their tumor-initiating and/or metastasis-promoting capabilities.

Certain embodiments include a kit for assaying ASPM levels for evaluating risk, presence, stage, or severity of pancreatic cancer, wherein the kit comprises a reagent capable of detecting ASPM levels in a biological sample of a subject and a test substrate; and instructions for contacting the reagent or substrate with a sample from the subject and instructions for evaluating the risk, predisposition, or prognosis for pancreatic cancer in a subject, wherein increased ASPM levels indicate an increased risk, an increased predisposition, or a poor prognosis.

Additional objects and advantages will be set forth in part in the description which follows, and in part will be obvious from the description, or may be learned by practice of aspects of the disclosure. The disclosed objects and advantages will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

FIG. 1 includes several panels relating to the structural organization of pancreatic epithelial cells using the three-dimensional culture model. Shown are representative confocal images of HPDE cell clusters (formed at 48 hours in culture; i, ii, v, vi) and tubules (formed at day 6 in culture; iii, iv, vii, viii) in three-dimensional reconstituted basement membrane matrices. The structures were immunostained with the basal surface marker α6-integrin (red) and the adheren junction marker β-catenin (green). Nuclei were counterstained with DAPI (blue). Asterisks: cell-free lumen. Insets: representative histological sections (H & E; 400× magnification) of a low-grade human PDAC tissue. Scale bars, 100 μm.

FIG. 2 includes several panels relating to the molecular alterations related to HPDE tubular morphogenesis and structural differentiation. (A) shows expression patterns of 620 differentially expressed genes (DEGs) during HPDE tubular morphogenesis. Also shown are their expression patterns in PANC-1 cellular clusters or spheroids. The heat map depicts high (red) and low (green) relative levels of medium-centered gene expression in log space. (B) shows fold changes in the transcript levels of CEL, CA9, MUC1, AGR2, and MUC20 as measured by qRT-PCR analysis. (C) shows Western blot analysis of lipase, carbonic anhydrase 9 or mucin-1 in HPDE or PANC-1 organoids. β-tubulin was included as a loading control.

FIG. 3 shows selection of the 28-gene gene set with the highest concordance index (C-index) for the prediction of post-operative survival of patients with PDAC in the UCSF cohort. Genes in the set of 620 differentially expressed genes during HPDE tubular morphogenesis were ranked-ordered according to the Cox's regression P-value. Multiple sets of genes were generated by repeatedly adding one more genes each time from top of the descendingly ranked list, starting from the first three top-ranked genes. For each selected probe set the C-index was used to evaluate the predictive accuracy in the survival analysis. C-index statistics analysis was conducted using the ‘survcomp’ package in the statistical programming language R (cran.r-project.org).

FIG. 4 shows Kaplan-Meier survival curves comparing post-operative survival in three independent cohorts (the UCSF cohort, the JHMI cohort, and the NW/NSU cohort) of patients with localized PDAC. The patients were stratified into two groups based on predicted risk of relapse (risk score; RS) calculated by the 28-gene prognostic signature described in Example 2. P values were calculated using the log-rank test. Shown on right are hazard ratios (with 95% confidence limits) of death according to the RS and clinico-pathological criteria in a Cox proportional-hazards analysis. *, P<0.05; **, P<0.01.

FIG. 5 shows Kaplan-Meier survival curves comparing overall survival of patients with PDAC in the UCSF cohort. The patients were stratified into two groups based on the transcript abundance levels of selected top-ranked (Cox regression P<0.01) gene markers in TABLE 2. Cut-off value that best discriminates between groups with respect to outcome was determined according to the maximal Youden's index. P values were calculated using the log-rank test.

FIG. 6 shows Kaplan-Meier survival curves comparing overall survival of patients with PDAC in the UCSF, JHMI, and NW/NSU cohorts. The patients were stratified into two groups based on the transcript abundance levels of ASPM. Cut-off value that best discriminates between groups with respect to outcome was determined according to the maximal Youden's index. P values were calculated using the log-rank test.

FIG. 7 includes several panels relating to the expression level of ASPM in pancreatic tissues and PDAC cell lines. (A) shows box plots of relative transcript levels of ASPM in microdissected normal pancreatic ducts (n=11) and PDAC tissues (n=11) interrogated using Oncomine (https://www.oncomine.com/resource/login.html). *, P<0.05. (B) shows the transcript levels of ASPM in HPDE cells and various PDAC cell lines as measured by qRT-PCR analysis. Data are represented as mean±SEM. n=3. **, P<0.01; ***, P<0.001.

FIG. 8 includes several panels relating to the functional importance of ASPM in PDAC cell proliferation and migration. (A) shows effect of shRNA-mediated silencing of ASPM in AsPC-1 or PANC-1 cells by Western blot analysis. β-tubulin was included as a loading control. (B) shows the rate of growth of control or ASPM shRNA-transduced AsPC-1 or PANC-1 cells. n=3. *, P<0.05; ***, P<0.001 versus control shRNA cells. (C) shows silencing of ASPM expression attenuated the migratory capacity of AsPC-1 or PANC-1 cells in response to pancreatic stellate cells in a modified Boyden chamber assay. n=3. *, P<0.05; ***, P<0.001.

FIG. 9 includes several panels relating to the role of ASPM in pancreatic cancer aggressiveness in vivo. (A) shows representative bioluminescence images (BLI) of NOD-SCID mice implanted in the pancreatic tails with ffLuc-labeled, control or ASPM shRNA-transduced AsPC-1 cells at the indicated time points following cell implantation. (B) shows tumor bulk quantified as BLI normalized photon counts as a function of time. Data are represented as mean±SEM. n=6. *, P<0.05; **, P<0.01. (C) shows the amounts of ascites in mice implanted with control shRNA-transduced AsPC-1 cells or ASPM shRNA-transduced cells measured at 6 weeks following cell implantation. n=3. **, P<0.01. (D) shows percent survival as a function of time in mice described in (A). P values were calculated using the log-rank test.

FIG. 10 includes several panels relating to the role of ASPM in the Wnt signaling pathway. (A) shows enrichment plot of Gene Set Enrichment Analysis showing that the KEGG Wnt signaling pathway was enriched in the differential gene expression profile of ASPM shRNA versus control shRNA transduced AsPC-1 cells. (B) shows fold Wnt-mediated luciferase expression in control or ASPM shRNA-transduced AsPC-1 cells. The luciferase activity of the cells was measured relative to basal activity 16 hours following treatment of the cells with Wnt-3a or vehicle. Data are represented as mean±SEM. n=3. ***, P<0.001.

FIG. 11 includes several panels relating to the role of ASPM in regulation β-catenin. (A) shows Western blot analysis on the protein abundance of β-catenin in control—or ASPM shRNA-transduced AsPC-1 or PANC-1 cells. β-tubulin was used as a loading control. (B) shows fold changes in the population doubling rate (left) and migration (right) of AsPC-1 cells that were transduced with the control or ASPM shRNA with or without co-expression of the S33Y β-catenin mutant. n=3. **, P<0.05; ***, P<0.001 versus control shRNA cells.

FIG. 12 includes several panels relating to the role of ASPM in pancreatic cancer stem cells. (A) shows Gene Set Enrichment Analysis showing significant enrichment of a core embryonic stem cell-like module gene set in the differential gene expression profile of ASPM-deficient versus control AsPC-1 cells. (B) shows representative plots showing patterns of CD44 and CD24 staining of AsPC-1 cells expressing the ASPM shRNA or control shRNA, with the frequency of the boxed CD44⁺CD24⁺ cell population as a percentage of cancer cells shown. (C) shows the mean (±SEM) percentages of CD44⁺CD24⁺ cell population from three independent measurements. *, P<0.05. (D) shows representative phase contrast images of tumorspheres formed by control- or ASPM-shRNA-transduced CD44⁺CD24^{low/-} AsPC-1 cells. Bars, 100 μm. (E) Bar graphs showing diameters of tumorspheres in (D). **, P<0.01.

FIG. 13 shows Kaplan-Meier survival curves comparing post-operative survival in three independent cohorts (the UCSF cohort, the JHMI cohort, and the NW/NSU cohort) of patients with localized PDAC. The patients were stratified into two groups based on predicted risk of relapse (risk score; RS) calculated by a 12-gene (ATP9A, ASPM, ACOX3, CDC45L, SLC40A1, AGR2, ATP11C, FAM72A, PLA2G10, MATN2, APITD1, and KIF11) prognostic signature described in Example 7. P values were calculated using the log-rank test.

FIG. 14 shows Kaplan-Meier survival curves comparing post-operative survival in three independent cohorts (the UCSF cohort, the JHMI cohort, and the NW/NSU cohort) of patients with localized PDAC. The patients were stratified into two groups based on predicted risk of relapse (risk score; RS) calculated by a six-gene (ASPM, ATP9A, ACOX3, CDC45L, SLC40A1, and AGR2) prognostic signature described in Example 8. P values were calculated using the log-rank test.

FIG. 15 shows Kaplan-Meier survival curves comparing post-operative survival in three independent cohorts (the UCSF cohort, the JHMI cohort, and the NW/NSU cohort) of patients with localized PDAC. The patients were stratified into two groups based on predicted risk of relapse (risk score; RS) calculated by a three-gene (ASPM, ATP9A, and ACOX3) prognostic signature described in Example 9. P values were calculated using the log-rank test.

FIG. 16 shows the transcript levels of ASPM in multiple breast cancer transcriptome data sets queried from Oncomine (www.oncomine.org) (Curtis et al., 2012; Ma et al., 2009; Richardson et al., 2006). ***, P<0.001 vs. normal. DCIS, ductal carcinoma in situ; IDC, invasive ductal carcinoma; ILC, invasive lobular carcinoma; TCGA, The Cancer Genome Atlas.

FIG. 17 shows Kaplan-Meier survival curves comparing overall or relapse-free survival in different large cohorts of patients with breast cancer (Curtis et al., 2012; Pawitan et al., 2005; Wang et al., 2005). The patients were grouped into quartiles according to the transcript abundance levels of ASPM. The log-rank test was used to calculate the P value.

FIG. 18 includes several panels relating to the functional importance of ASPM in breast cancer cell proliferation, migration and Wnt activity. (A) shows effect of shRNA-mediated silencing of ASPM in breast cancer HCC-1954 cells by Western blot analysis. β-tubulin was included as a loading control. (B) shows the rate of growth of control or ASPM shRNA-transduced MDA-MD-436 or HCC-1954 cells. n=3. **, P<0.01; ***, P<0.001 versus control shRNA cells. (C) shows silencing of ASPM expression attenuated the migratory capacity of MDA-MD-436 or HCC-1954 cells in response to primary breast carcinoma-associated fibroblasts in a modified Boyden chamber assay. n=3. ***, P<0.001. (D) shows Wnt-mediated luciferase expression in control—or ASPM-shRNA-transduced MDA-MD-436 or HCC-1954 cells. Data are represented as mean±SEM (n=3). *, P<0.05 versus control shRNA.

FIG. 19 includes several panels relating to the role of ASPM in breast cancer stem cells. (A) shows representative plots showing patterns of CD44 and CD24 staining of MDA-MB-436 cells expressing the ASPM shRNA or control shRNA, with the frequency of the boxed CD44⁺CD24^{-/low} cell population as a percentage of cancer cells shown. (B) shows the mean (±SEM) percentages of CD44+CD24^{-/low} cell population from three independent measurements. ***, P<0.001. (C) shows representative phase contrast images of tumorspheres formed by control—or ASPM-shRNA-transduced CD44⁺CD24^{low/-} MDA-MB-436 cells. Bars, 100 μm. (D) Bar graphs showing diameters of tumorspheres in (C). ***, P<0.001.

FIG. 20 includes several panels relating to the role of ASPM in breast tumorigenesis in vivo. (A) shows representative bioluminescence images (BLI) of NOD-SCID mice implanted in the mammary fat pads with firefly luciferase-labeled, control or ASPM shRNA-transduced breast cancer MDA-MB-436 cells at the indicated time points following cell implantation. (B) shows tumor bulk quantified as BLI normalized photon counts as a function of time. Data are represented as mean±SEM (n=6 in each group). *, P<0.05; ***, P<0.001 vs. Control shRNA.

FIG. 21 includes several panels relating to the expression level of ASPM in human prostate cancer tissues. (A) shows the transcript levels of ASPM in normal prostate (n=9) and prostate cancer tissues (n=73) in human Tissue cDNA Arrays (Origene) as measured by quantitative reverse transcriptase polymerase chain reaction analysis. GS, Gleason score. ***, P<0.001 vs. normal tissues. (B) shows the transcript levels of ASPM in primary and metastatic prostate cancers in multiple transcriptome data sets queried from Oncomine (www.oncomine.org) (Chandran et al., 2007; Grasso et al., 2012; Varambally et al., 2005). **, P<0.01; ***, P<0.001 vs. primary prostate cancer.

FIG. 22 includes several panels relating to the functional importance of ASPM in prostate cancer cell proliferation, migration and Wnt activity. (A) shows effect of shRNA-mediated silencing of ASPM in prostate cancer PC-3 cells by Western blot analysis. β-tubulin was included as a loading control. (B) shows the rate of growth of control or ASPM shRNA-transduced PC-3 cells. n=3. **, P<0.01; ***, P<0.001 versus control shRNA cells. (C) shows silencing of ASPM expression attenuated the migratory capacity of PC-3 in response to serum-containing growth medium in a modified Boyden chamber assay. n=3. ***, P<0.001. (D) shows Wnt-mediated luciferase expression in control—or ASPM-shRNA-transduced PC-3 cells. Data are represented as mean±SEM (n=3). ***, P<0.001 vs. control shRNA.

FIG. 23 includes several panels relating to the role of ASPM in prostate cancer stem cells. (A) shows representative plots showing patterns of CD133 and CD44 staining of PC-3 cells expressing the ASPM shRNA or control shRNA, with the frequency of the boxed CD133⁺CD44⁺ cell population as a percentage of cancer cells shown. (B) shows the mean (±SEM) percentages of CD133⁺CD44⁺ cell population from three independent measurements. **, P<0.01 vs. control shRNA.

### DETAILED DESCRIPTION

The present disclosure includes methods of diagnosing the degree of differentiation and predicting clinical prognosis of pancreatic cancer by examining molecular markers (either the protein or the RNA encoding the protein), including ATP9A, ASPM, ACOX3, CDC45L, SLC40A1, AGR2, and those found in TABLE 2, or a combination thereof, including wild-type, truncated or alternatively spliced forms, in biological samples obtained from any subject having pancreatic tissues suspected of being or known to be cancerous, e.g. pancreatic cancer tissue. The methods provided in the disclosure have enabled, among other things, the prediction of clinical prognosis, including disease recurrence, metastasis, treatment response, and overall survival in any subject with pancreatic cancer. Accordingly, the certain embodiments can be used to screen subjects with pancreatic cancer for poor clinical prognosis, including, for example, disease recurrence following treatments, which can direct treatment decisions and the choice of treatment modalities for subjects with pancreatic cancer. Thus, the subject (e.g., a pancreatic cancer patient) and the caregiver can make better informed decisions of whether or not to perform surgery (e.g., radical pancreaticctomy), neo-adjuvant (i.e., before surgery), adjuvant therapy (i.e., after surgery), including, without limitation, radiation treatment, chemotherapy treatment, treatment with biological agents, or hormone therapy, and/or other alternate treatment(s).

Disclosed methods involve determining the level of a polypeptide or polynucleotide in a patient and then comparing the level to a threshold reference or range. Typically, the threshold reference value is representative of a polypeptide or polynucleotide in a large number of persons or tissues with pancreatic cancer and whose clinical prognosis data are available, as measured using a tissue sample or biopsy or other biological sample such as a cell, serum or blood. Said threshold reference values are determined by defining levels wherein said subjects whose tumors have expression levels of said markers above said threshold reference level(s) are predicted as having a higher or lower degree of differentiation or risk of poor clinical prognosis or disease progression than those with expression levels below said threshold reference level(s). Variation of levels of a polypeptide or polynucleotide from the reference range (either up or down) indicates that the patient has a higher or lower degree of differentiation or risk of poor clinical prognosis or disease progression than those with expression levels below said threshold reference level(s).

In certain embodiments, the method includes obtaining a measurement of the transcript or protein expression levels of one or more marker genes in one or more tumor samples from a subject. In certain embodiments, tumor samples can be obtained by the methods of aspiration, biopsy, or surgical resection. In certain embodiments, the tumor sample may be a fresh sample, a frozen sample, or a fixed, wax-embedded sample.

The methods of predicting clinical prognosis of subjects with pancreatic cancer based on the expression levels of ASPM, ATP9A, ACOX3, CDC45L, SLC40A1, AGR2, and those found in TABLE 2, can also involve the use of statistical methods, including, without limitation, class distinction using unsupervised methods (e.g., k-means, hierarchical clustering, principle components, non-negative matrix factorization, or multidimensional scaling) (Hastie et al., 2009), supervised methods (e.g., discriminant analysis, support vector machines, or k-nearest-neighbors) or semi-supervised methods, or outcome prediction (e.g., relapse-free survival, disease progression, or overall survival) using Cox regression model (Kalbfleisch and Prentice, 2002), accelerated failure time model, Bayesian survival model, or smoothing analysis for survival data (Wand, 2003).

In certain embodiments, methods of diagnosing the degree of differentiation and predicting clinical prognosis of pancreatic cancer involve determining in a biological sample from a subject with pancreatic cancer the expression level of one or more of the gene markers including ASPM, ATP9A, ACOX3, CDC45L, SLC40A1, AGR2, and those disclosed in TABLE 2. The markers useful in a disclosed method include any individual marker in TABLE 2, or any combination of two or more markers thereof (e.g., any two, three, four, five, six, seven, eight, nine, 10, 11, 12, 13, 14, 15, 16, 17, 18, 19, 20, 21, 22, 23, 24, 25, 26, 27 or all 28 of the markers in TABLE 2, or at least two, at least three, at least four, at least five, at least six, at least seven, at least eight, at least nine, at least 10, at least 11, at least 12, at least 13, at least 14, at least 15, at least 16, at least 17 at least 18 at least 19 at least 20, at least 21, at least 22, at least 23, at least 24, at least 25, at least 26, or at least 27 of the markers in TABLE 2).

Certain embodiments relate to the prediction of pancreatic cancer prognosis by determining the protein expression levels of ASPM, ATP9A, or ACOX3 or any combination thereof (e.g, any two, or all of the three markers, or at least two of these markers) in a biological sample from a subject with pancreatic cancer.

Certain embodiments relate to the prediction of pancreatic cancer prognosis by determining the protein expression levels of ASPM, ATP9A, ACOX3, CDC45L, SLC40A1, or AGR2 or any combination thereof (e.g, any two, any three, any four, any five or all of the six markers, or at least two, at least three, at least four, or at least five of these markers) in a biological sample from a subject with pancreatic cancer.

Certain embodiments relate to the prediction of pancreatic cancer prognosis by determining the protein expression levels of ASPM, ATP9A, ACOX3, CDC45L, SLC40A1, AGR2, ATP11C, FAM72A, PLA2G10, MATN2, APITD1, or KIF11 or any combination thereof (e.g, any two, any three, any four, any five, any six, any seven, any eight, any nine, any 10, any 11 or all of the 12 markers, or at least two, at least three, at least four, at least five, at least six, at least seven, at least eight, at least nine, at least 10, or at least 11 of these markers) in a biological sample from a subject with pancreatic cancer.

In certain embodiments, a predicted clinical prognosis can include changes in the number, size, or volume of one or a plurality of measurable tumor lesions. In certain embodiments, assessing or evaluating the number, size, or volume of tumor lesions can include visual, radiological, and/or pathological examination of a tumor or pancreatic cancer before or at various time points during and after diagnosis or surgery.

In exemplary methods, determining the protein expression levels comprises the use of antibodies specific to said gene markers and immunohistochemistry staining on fixed (e.g., formalin-fixed) and/or wax-embedded (e.g., paraffin-embedded) pancreatic tumor tissues. Fixatives for tissue preparations or cells are well known in the art and include formalin, gluteraldehyde, methanol, or the like (Carson, Histotechology: A Self-Instructional Text, Chicago: ASCP Press, 1997). The immunohistochemistry methods may be performed manually or in an automated fashion.

Antibody reagents can be used in assays to detect expression levels of ASPM, ATP9A, ACOX3, CDC45L, SLC40A1, AGR2, and/or those found in TABLE 2 in patient samples using any of a number of immunoassays known to those skilled in the art. Immunoassay techniques and protocols are generally described in Price and Newman, “Principles and Practice of Immunoassay,” 2nd Edition, Grove's Dictionaries, 1997; and Gosling, “Immunoassays: A Practical Approach,” Oxford University Press, 2000. A variety of immunoassay techniques, including competitive and non-competitive immunoassays, can be used. See, e.g., Self et al., Curr. Opin. Biotechnol., 7:60-65 (1996). The term immunoassay encompasses techniques including, without limitation, enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with laser induced fluorescence. See, e.g., Schmalzing et al., Electrophoresis, 18:2184-93 (1997); Bao, J. Chromatogr. B. Biomed. Sci., 699:463-80 (1997). Liposome immunoassays, such as flow-injection liposome immunoassays and liposome immunosensors, are also suitable for use in certain embodiments. See, e.g., Rongen et al., J. lmmunol. Methods, 204:105-133 (1997). In addition, nephelometry assays, in which the formation of protein/antibody complexes results in increased light scatter that is converted to a peak rate signal as a function of the marker concentration, are suitable for use in the methods certain embodiments. Nephelometry assays are commercially available from Beckman Coulter (Brea, Calif.; Kit #449430) and can be performed using a Behring Nephelometer Analyzer (Fink et al., J. Clin. Chem. Clin. Biochem., 27:261-276 (1989)).

Specific immunological binding of the antibody to nucleic acids can be detected directly or indirectly. Direct labels include fluorescent or luminescent tags, metals, dyes, radionuclides, and the like, attached to the antibody. An antibody labeled with iodine-125 (¹²⁵I) can be used. A chemiluminescence assay using a chemiluminescent antibody specific for the nucleic acid is suitable for sensitive, non-radioactive detection of protein levels. An antibody labeled with fluorochrome is also suitable. Examples of fluorochromes include, without limitation, DAPI, fluorescein, Hoechst 33258, R-phycocyanin, B-phycoerythrin, R-phycoerythrin, rhodamine, Texas red, and lissamine. Indirect labels include various enzymes well known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-β-D-galactopyranoside (ONPG), which yields a soluble product detectable at 410 nm. An urease detection system can be used with a substrate such as urea-bromocresol purple (Sigma Immunochemicals; St. Louis, Mo.).

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis can be made using a spectrophotometer such as an EMAX Microplate Reader (Molecular Devices; Menlo Park, Calif.) in accordance with the manufacturer's instructions. If desired, the assays of certain embodiments can be automated or performed robotically, and the signal from multiple samples can be detected simultaneously.

The antibodies can be immobilized onto a variety of solid supports, such as magnetic or chromatographic matrix particles, the surface of an assay plate (e.g., microtiter wells), pieces of a solid substrate material or membrane (e.g., plastic, nylon, paper), in the physical form of sticks, sponges, papers, wells, and the like. An assay strip can be prepared by coating the antibody or a plurality of antibodies in an array on a solid support. This strip can then be dipped into the test sample and processed quickly through washes and detection steps to generate a measurable signal, such as a colored spot.

Alternatively, nucleic acid binding molecules such as probes, oligonucleotides, oligonucleotide arrays, and primers can be used in assays to detect differential RNA expression of ASPM, ATP9A, ACOX3, CDC45L, SLC40A1, AGR2, and/or those found in TABLE 2 in patient samples, e.g., RT-PCR. In one embodiment, RT-PCR is used according to standard methods known in the art. In another embodiment, PCR assays such as Taqman® assays available from, e.g., Applied Biosystems, can be used to detect nucleic acids and variants thereof. In other embodiments, qPCR and nucleic acid microarrays can be used to detect nucleic acids. Reagents that bind to selected cancer biomarkers can be prepared according to methods known to those of skill in the art or purchased commercially.

Analysis of nucleic acids can be achieved using routine techniques such as Southern analysis, reverse-transcriptase polymerase chain reaction (RT-PCR), or any other methods based on hybridization to a nucleic acid sequence that is complementary to a portion of the marker coding sequence (e.g., slot blot hybridization) are also within the scope of certain embodiments. Applicable PCR amplification techniques are described in, e.g., PCR Protocols: A Guide to Methods and Applications (Innis et al, eds, 1990). General nucleic acid hybridization methods are described in Anderson, “Nucleic Acid Hybridization,” BIOS Scientific Publishers, 1999. Amplification or hybridization of a plurality of nucleic acid sequences (e.g., genomic DNA, mRNA or cDNA) can also be performed from mRNA or cDNA sequences arranged in a microarray. Microarray methods are generally described in Hardiman, “Microarrays Methods and Applications: Nuts & Bolts,” DNA Press, 2003; and Baldi et al., “DNA Microarrays and Gene Expression: From Experiments to Data Analysis and Modeling,” Cambridge University Press, 2002.

Analysis of nucleic acid markers and their variants can be performed using techniques known in the art including, without limitation, microarrays, polymerase chain reaction (PCR)-based analysis, sequence analysis, and electrophoretic analysis. A non-limiting example of a PCR-based analysis includes a Taqman® allelic discrimination assay available from Applied Biosystems. Non-limiting examples of sequence analysis include Maxam-Gilbert sequencing, Sanger sequencing, capillary array DNA sequencing, thermal cycle sequencing (Sears et al., Biotechniques, 13:626-633 (1992)), solid-phase sequencing (Zimmerman et al., Methods Mol. Cell BioL, 3:39-42 (1992)), sequencing with mass spectrometry such as matrix-assisted laser desorption/ionization time-of-flight mass spectrometry (MALDI-TOF/MS; Fu et al., Nat. Biotechnol., 16:381-384 (1998)), and sequencing by hybridization. Chee et al., Science, 274:610-614 (1996); Drmanac et al., Science, 260:1649-1652 (1993); Drmanac et al., Nat. Biotechnol., 16:54-58 (1998). Non-limiting examples of electrophoretic analysis include slab gel electrophoresis such as agarose or polyacrylamide gel electrophoresis, capillary electrophoresis, and denaturing gradient gel electrophoresis. Other methods for detecting nucleic acid variants include, e.g., the INVADER® assay from Third Wave Technologies, Inc., restriction fragment length polymorphism (RFLP) analysis, allele-specific oligonucleotide hybridization, a heteroduplex mobility assay, single strand conformational polymorphism (SSCP) analysis, single-nucleotide primer extension (SNUPE) and pyrosequencing.

A detectable moiety can be used in the assays described herein. A wide variety of detectable moieties can be used, with the choice of label depending on the sensitivity required, ease of conjugation with the antibody, stability requirements, and available instrumentation and disposal provisions. Suitable detectable moieties include, but are not limited to, radionuclides, fluorescent dyes (e.g., fluorescein, fluorescein isothiocyanate (FITC), Oregon Green™, rhodamine, Texas red, tetrarhodimine isothiocynate (TRITC), Cy3, Cy5, etc.), fluorescent markers (e.g., green fluorescent protein (GFP), phycoerythrin, etc.), autoquenched fluorescent compounds that are activated by tumor-associated proteases, enzymes (e.g., luciferase, horseradish peroxidase, alkaline phosphatase, etc.), nanoparticles, biotin, digoxigenin, and the like.

Useful physical formats comprise surfaces having a plurality of discrete, addressable locations for the detection of a plurality of different markers. Such formats include microarrays and certain capillary devices. See, e.g., Ng et al., J. Cell Mol. Med., 6:329-340 (2002); U.S. Pat. No. 6,019,944. In these embodiments, each discrete surface location may comprise antibodies to immobilize one or more markers for detection at each location. Surfaces may alternatively comprise one or more discrete particles (e.g., microparticles or nanoparticles) immobilized at discrete locations of a surface, where the microparticles comprise antibodies to immobilize one or more markers for detection. Other useful physical formats include sticks, wells, sponges, and the like.

Analysis can be carried out in a variety of physical formats. For example, the use of microtiter plates or automation can be used to facilitate the processing of large numbers of test samples. Alternatively, single sample formats could be developed to facilitate diagnosis or prognosis in a timely fashion.

Alternatively, the antibodies or nucleic acid probes of certain embodiments can be applied to patient samples immobilized on microscope slides. The resulting antibody staining or in situ hybridization pattern can be visualized using any one of a variety of light or fluorescent microscopic methods known in the art.

Analysis of the protein or nucleic acid can also be achieved, for example, by high pressure liquid chromatography (HPLC), alone or in combination with mass spectrometry (e.g., MALDI/MS, MALDI-TOF/MS, tandem MS, etc.).

### Exemplary Molecular Markers

1. ATPase, class II, type 9A (ATP9A)

The human ATPase, class II, type 9A (ATP9A) gene (NCBI Entrez Gene 10079) is located on chromosome 20 at gene map locus 20q13.1 and encodes 912 amino acids. This gene's function is still unclear, and there is only one splice form. Exemplary ATP9A sequences are publically available, for example from GenBank (e.g., accession numbers NM_{—}006045.1 (mRNA) and NP_{—}006036.1 (protein)), or UniProtKB (e.g., Q2NLD0).

2. Asp (abnormal spindle) homolog, microcephaly associated (ASPM)

The human Asp (abnormal spindle) homolog, microcephaly associated (ASPM) gene (NCBI Entrez Gene 259266) is the human ortholog of the Drosophila melanogaster ‘abnormal spindle’ gene (asp), which is located on chromosome 1 at gene map locus 1q31 and molecular mass of 410 kD. The role of this gene is essential for normal mitotic spindle function in embryonic neuroblasts and mitotic spindle regulation. Two alternative splice variants have been identified. ASPM sequences are publically available, for example form GenBank (e.g., accession numbers NM_{—}001206846.1, and NM_{—}018136.4 (mRNAs) and NP_{—}001193775.1, and NP_{—}060606.3 (proteins)), or UniProtKB (e.g., Q8IZT6).

3. Acyl-Coenzyme A oxidase 3, pristanoyl (ACOX3)

The human Acyl-Coenzyme A oxidase 3, pristanoyl (ACOX3) gene (NCBI Entrez Gene 8310) is located on chromosome 4 at map locus 4p15.3. ACOX3 is involved in the desaturation of 2-methyl branched fatty acids in peroxisomes. It is suggested that the enzyme is expressed only under special situations, such as during particular developmental stages, or in specialized tissues. ACOX3 has two alternative splice variants. Exemplary ACOX3 sequences are publically available, for example form GenBank (e.g., accession numbers NM_{—}001101667.1, and NM_{—}003501.2 (mRNAs) and NP_{—}001095137.1, and NP_{—}003492.2 (proteins)), or UniProtKB (e.g., O15254).

4. CDC45 cell division cycle 45-like (CDC45L)

The human CDC45 cell division cycle 45 (CDC45L) gene (NCBI Entrez Gene 259266) is located on chromosome 22 at map locus 22q11.21. CDC45L is a member of the highly conserved multiprotein complex including Cdc6/Cdc18, the minichromosome maintenance proteins (MCMs) and DNA polymerase, which is important for early steps of DNA replication in eukaryotes. Multiple alternatively spliced transcript variants encoding different isoforms have been found for CDC45L. Exemplary CDC45L sequences are publically available, for example from GeneBank (e.g., accession numbers NM_{—}001178010.1, NM_{—}001178011.1, and NM_{—}003504.3 (mRNAs) and NP_{—}001171481.1, NP_{—}001171482.1, and NP_{—}003495.1 (proteins)), or UniProtKB (e.g., O75419).

5. Solute carrier family 40 (iron-regulated transporter), member 1 (SLC40A1)

The human Solute carrier family 40 (iron-regulated transporter), member 1 (SLC40A1) gene (NCBI Entrez Gene 30061) is located on chomosome 2 at gene map locus 2q32. The SLC40A1 gene encodes a cell membrane protein that may be involved in iron export from duodenal epithelial cells and is up-regulated in the iron overload disease hereditary hemochromatosis. Only one splice form has been identified. Exemplary SLC40A1 sequences are publically available, for example from GenBank (e.g., accession numbers NM_{—}014585.5 (mRNA) and NP_{—}997512.1 (protein)), or UniProtKB (e.g., Q9NP59).

6. Anterior gradient homolog 2 (AGR2)

The human Anterior gradient homolog 2 (AGR2) gene (NCBI Entrez Gene 10551) is located on chromosome 7 at map locus 7p21.3. AGR2 mRNA and protein exhibits similar expression patterns in breast cancer tissues. Expression of AGR2 shows a positive correlation with expression of estrogen receptor and a negative correlation with expression of EGF receptor. Exemplary AGR2 sequences are publically available, for example from GenBank (e.g., accession numbers NM_{—}006408.3 (mRNA) and NP_{—}006399.1 (protein)), or UniProtKB (e.g., Q4JM47).

7. ATPase, class VI, type 11C (ATP11C)

The human ATPase, class VI, type 11C (ATP11C) gene (NCBI Entrez Gene 10079) is located on chromosome X at gene map locus Xq27.1 and encodes 1132 amino acids. This gene's function is still unclear. Two alternative splice forms have been identified. Exemplary ATP11C sequences are publically available, for example form GenBank (e.g., accession numbers NM_{—}001010986.2, and NM_{—}173694.4 (mRNA) and NP_{—}001010986.1, and NP_{—}775965.2 (protein)) or UniProtKB (e.g., Q8NB49).

8. Family with sequence similarity 72, member A (FAM72A)

The family with sequence similarity 72, member A (FAM72A) gene (NCBI Entrez Gene 729533) is the human ortholog of the family with sequence similarity 72, member A, which is located on chromosome 1 at gene map locus 1p11. The FAM72A gene encodes a protein with a molecular mass of 149 kD. FAM72A is upregulated in several common cancers compared with matched normal tissues. Only one splice form of FAM72A has been identified. Exemplary FAM72A sequences are publically available, for example form GenBank (e.g., accession numbers NM_{—}00123168.1 (mRNA) and NP_{—}001116640.1 (protein)) or UniProtKB (e.g., Q5TYM5).

9. Phospholipase A2, group X (PLA2G10)

The human phospholipase A2, group X (PLA2G10) gene (NCBI Entrez Gene 8399) is located on chomosome 16 at gene map locus 16p13.12 and encodes a protein consisting of 42 amino acids. The function of the PLA2G10 gene is still unclear, and only one splice form has been identified. Exemplary ATP9A sequences are publically available, for example form GenBank (e.g., accession numbers NM_{—}003561.1 (mRNA) and NP_{—}003552.1(protein)) or UniProtKB (e.g., O15496).

10. Matrilin 2 (MATN2)

The human Matrilin 2 (MATN2) gene (NCBI Entrez Gene 4147) is located on chromosome 8 at gene map locus 8q22 and encodes a protein consisting of 956 amino acids. Two mRNA transcripts of the MATN2 gene have been identified. Exemplary MATN2 sequences are publically available, for example form GenBank (e.g., accession numbers NM_{—}002380.3, and NM_{—}030583.2 (mRNA) and NP_{—}002371.3, and NP_{—}085072.2 (protein)) or UniProtKB (e.g., O00339).

11. Apoptosis-inducing, TAF9-like domain 1 (APITD1)

The human apoptosis-inducing, TAF9-like domain 1 (APITD1) gene (NCBI Entrez Gene 378708) is identified in the neuroblastoma tumor suppressor candidate region on chromosome 1p36. It contains a TFIID-31 domain, similar to that found in TATA box-binding protein-associated factor, TAF(II)31, which is required for p53-mediated transcription activation. This gene is expressed at very low levels in neuroblastoma tumors, and was shown to reduce cell growth in neuroblastoma cells, suggesting that it may have a role in a cell death pathway. Multiple alternatively spliced transcript variants have been identified. Exemplary APITD1 sequences are publically available, for example form GenBank (e.g., accession numbers NM_{—}001270517.1, NM_{—}198544.3, NM_{—}199006.2 and NM_{—}001243768.1 (mRNAs) and NP_{—}001257446.1, NP_{—}940946.1, and NP_{—}950171.2, and NP_{—}001230697.1 (proteins)) or UniProtKB (e.g., H2PXZ6).

12. Kinesin family member 11 (KIF11)

The human Kinesin family member 11(KIF11) gene (NCBI Entrez Gene 3832) is located on chromosome 10 at gene map locus 10q24.1. KIF11 encodes a motor protein that belongs to the kinesin-like protein family. Members of this protein family are known to be involved in various kinds of spindle dynamics. The function of KIF11 includes chromosome positioning, centrosome separation and establishing a bipolar spindle during cell mitosis. There is only one splice form of KIF11. Exemplary KIF11 sequences are publically available, for example from GenBank (e.g., accession numbers NM_{—}004523.3 (mRNA) and NP_{—}004514.2 (protein)) or UniProtKB (e.g., P52732).

### Exemplary Kits, Apparatuses, and Compositions

A. Kits

We contemplated kits useful for facilitating the practice of certain embodiments of the disclosed methods. In one embodiment, kits are provided for detecting one or more of the genes disclosed in TABLE 2 (such as, at least one, at least two, at least three, at least four, at least five, at least six, at least seven, at least eight, at least nine, at least 10, at least 11, at least 12, at least 13, at least 14, at least 15, at least 16, at least 17 at least 18 at least 19 at least 20, at least 21, at least 22, at least 23, at least 24, at least 25, at least 26, or at least 27 or all of the 28 genes disclosed in TABLE 2). In one embodiment, a kit is provided for detecting at least ASPM and ATP9 Anucleic acid or protein molecules, for example in combination with one to a plurality of housekeeping genes or proteins (e.g., β-actin, GAPDH, RPL13A, tubulin, and the like well known in the art of protein biochemistry). In yet another embodiment, a kit is provided for detecting at least ASPM, ATP9A, and ACOX3 nucleic acid or protein molecules, for example in combination with one to a plurality of housekeeping genes or proteins. The detectors or methods of detection can include detectors of a genomic alteration involving the gene and/or a gene expression product, such as an mRNA or protein. The detectors can include, without limitation, a nucleic acid probe specific for a genomic sequence including said disclosed gene, a nucleic acid probe specific for a transcript (e.g., mRNA) encoded by said gene, a pair of primers for specific amplification of said disclosed gene, an antibody or antibody fragment specific for a protein encoded by said disclosed gene, or an aptamers specific for a protein encoded by said disclosed genes. In a particular example, kits can include one or more (such as two, three, or four) detectors selected from a nucleic acid probe specific for ASPM transcript, a nucleic acid probe specific for ATP9A transcript, a nucleic acid probe specific for ACOX3 transcript, and nucleic acid probes specific for the transcripts of the other genes listed in TABLE 2, a pair of primers for specific amplification of ASPM, a pair of primers for specific amplification of ATP9A, a pair of primers for specific amplification of ACOX3, and pairs of primers for specific amplification of the transcripts of the other genes listed in TABLE 2, an antibody specific for ATP9A protein, an antibody specific for ASPM protein, an antibody specific for ACOX3 protein, and antibodies specific for the proteins encoded by the genes listed in TABLE 2. Particular kit embodiments can further include, for instance, one or more (such as two, three or four) detectors selected from a nucleic acid probe specific for a housekeeping transcript, a pair of primers for specific amplification of housekeeping transcript, and an antibody specific for one or more housekeeping protein.

In some embodiments, the primary detection means (e.g., nucleic acid probe, nucleic acid primers, or antibody) can be directly labeled with a fluorophore, chromophore, or enzyme capable of producing a detectable product (e.g., alkaline phosphates, horseradish peroxidase and others commonly known in the art). In other embodiments, kits are provided including secondary detection means, such as secondary antibodies or non-antibody hapten-binding molecules (e.g., avidin or streptavidin). In some such instances, the secondary detection means will be directly labeled with a detectable moiety. In other instances, the secondary or higher order antibody can be conjugated to a hapten (e.g., biotin, DNP, or FITC), which is detectable by a cognate hapten binding molecule (e.g., streptavidin horseradish peroxidase, streptavidin alkaline phosphatase, or streptavidin QDot™). Some kit embodiments can include colorimetric reagents in suitable containers to be used in concert with primary, secondary or higher order detection means that are labeled with enzymes for the development of such colorimetric reagents.

In one embodiment, kits include positive or negative control samples, such as nucleic acid samples that correspond or do not correspond to transcripts of the genes listed in TABLE 2, protein lysates that contain or do not contain proteins or fragmented proteins encoded by the genes listed in TABLE 2, and/or cell line or tissue known to express or not express a gene or gene product listed in TABLE 2.

Nucleic acid probes or primers used in the methods provided herein can be obtained from a commercially available source or prepared using techniques well known in the art. Nucleic acid probes and primers are nucleic acid molecules capable of hybridizing with a target nucleic acid molecule (e.g., genomic target nucleic acid molecule). For instance, probes specific to ASPM, ATP9A, ACOX3 or a gene listed in TABLE 2, when hybridized to the target, are capable of being detected either directly or indirectly. Primers specific for ASPM, ATP9A, ACOX3 or a gene listed in TABLE 2, when hybridized to the target, are capable of amplifying the target gene, and the resulting amplicons capable of being detected either directly or indirectly.

Antibodies or aptamers used in the methods provided here can be obtained from a commercially available source or prepared using techniques well known in the art. Antibodies are immunoglobulin molecules (or combinations thereof) that specifically bind to, or are immunologically reactive with, a particular antigen, and includes polyclonal, monoclonal, genetically engineered and otherwise modified forms of antibodies, including but not limited to chimeric antibodies, humanized antibodies, hetero-conjugate antibodies, single chain Fv antibodies, polypeptides that contain at least a portion of an immunoglobulin that is sufficient to confer specific antigen biding to the polypeptide, and antigen binding fragments of antibodies. Antibody fragments include proteolytic antibody fragments, recombinant antibody fragments, complementarity determining region fragments, camelid antibodies (e.g., U.S. Pat. Nos. 6,015,695; 6,005,079; 5,874,541; 5,840,526; 5,800,988; and 5,759,808), and antibodies produced by cartilaginous and bony fishes and isolated binding domains thereof.

Commercial sources of antibodies include Sigma-Aldrich (St. Louis, Mo., USA), Santa Cruz (Santa Cruz, Calif., USA), Abnova (Taipei, Taiwan), SDIX (Neward, Del., USA), EMD Millipore (Billerica, Mass., USA), GeneTex (Irvine, Calif., USA), Epitomics (Burlingame, Calif., USA), LSBio (Seattle, Wash., USA) and other antibody providers. TABLE 1 shows exemplary commercial sources of antibodies for ATP9A, ASPM, ACOX3, CDC45L, SLC40A1, and AGR2.

TABLE 1
Exemplary commercial sources of antibodies

| | Antibody | | Catalog | |
|---|---|---|---|---|
| Marker | type | Source | number | Application |
| ATP9A | Rabbit | Sigma- | HPA035630 | IHC, WB |
| | polyclonal | Aldrich | | |
| | Rabbit | Santa Cruz | sc-85287 | WB, IP, IF, |
| | monoclonal | Abnova | H00010079- | ELISA |
| | Mouse | | M02 | IF, ELISA, |
| | monoclonal | | | WB-Re |
| ASPM | Rabbit | SDIX | 2597.00.02 | IHC, IF |
| | monoclonal | | | |
| | Rabbit | Santa Cruz | sc-98903 | WB, IP, IF, |
| | monoclonal | EMD | 09-066 | ELISA |
| | Rabbit | Millipore | | IH, ELISA |
| | polyclonal | | | |
| ACOX3 | Rabbit | Santa Cruz | sc-98757 | WB, IP, IF, |
| | monoclonal | GeneTex | GTX115077 | ELISA |
| | Rabbit | | | IHC-P, WB |
| | polyclonal | | | |
| | Rabbit | Atlas | HPA035840 | IHC, WB |
| | polyclonal | | | |
| CDC45L | Rabbit | GeneTex | GTX109454 | ICC/IF, WB |
| | polyclonal | | | |
| | Rabbit | Epitomics | S1672 | WB, ICC |
| | Polyclonal | | | |
| | Mouse | LSBio | LS-C78972-50 | WB |
| | monoclonal | | | |
| SLC40A1 | Rabbit | GeneTex | GTX85744 | ELISA, IHC-P, |
| | Polyclonal | | | WB |
| | Rabbit | Abnova | PAB5460 | ELISA, WB |
| | Polyclonal | | | |
| | Rabbit | LSBio | LS-B1836-50 | ELISA, IHC-P, |
| | Polyclonal | | | WB |
| AGR2 | Mouse | Abnova | PAB12150 | WB, IHC-P |
| | Polyclonal | | | |
| | Mouse | Santa Cruz | sc-101211 | WB, IP, ELISA |
| | monoclonal | LSBio | LS-C122675- | ELISA, IHC, WB |
| | Sheep | | 100 | |
| | Polyclonal | | | |

Methods of generating antibodies (e.g., monoclonal or polyclonal antibodies) are well known in the art (e.g., Harlow and Lane, Antibodies: A Laboratory Manual, Cold Spring Harbor Laboratory, New York, 1988). For example, peptide fragments of one of the proteins listed in TABLE 2, such as ASPM, ATP9A, ACOX3, CDC45L, SLC40A1, or AGR2, can be conjugated to a carrier molecules (or nucleic acids encoding such epitopes) can be injected into non-human mammals (e.g., mice or rabbits), followed by boost injections, to produce an antibody response. Serum isolated from immunized animals may be isolated for the polyclonal antibodies contained therein, or spleens from immunized animals may be used for the production of hybridomas and monoclonal antibodies. Antibodies can be further purified before use.

Aptamers used in the methods disclosed herein include single stranded nucleic acid molecule (e.g., DNA or RNA) that assumes one or more particular, sequence-specific shapes and binds to one of the protein products of the genes listed in TABLE 2 with high affinity and specificity. In another example, an aptamer is a peptide aptamer that binds to one of the protein products of the genes listed in TABLE 2 with high affinity and specificity. Peptide aptamers include a peptide loop which is specific for the target protein attached at both ends to a protein scaffold. The scaffold may be any protein which is stable, soluble, small, and non-toxic. Peptide aptamer selection can be made using different systems, such as the yeast two-hybrid system or the Lex A interaction trap system.

In certain embodiments, a kit may include a carrier means, such as a box, a bag, a vial, a tube, a satchel, plastic carton, wrapper, or other container. In some examples, kit components will be enclosed in a single packing unit, which may have compartments into which one or more components of the kit can be placed. In other examples, a kit includes one or more containers that can retain, for example, one or more biological samples to be tested. In some embodiments, a kit may include buffers and other reagents that can be used for the practice of a particular disclosed method. Such kits and appropriate contents are well known to those skilled in the art.

B. Arrays

Microarrays useful for facilitating the practice of a disclosed method are contemplated. Microarrays for the detection of genes or proteins are well known in the art. Microarrays include a solid surface (e.g., glass slide) upon which many (e.g., hundreds or thousands) of specific binding agents (e.g., cDNA probes, mRNA probes, or antibodies) are immobilized. The specific binding agents are distinctly located in an addressable (e.g., grid) format on the array. The specific binding agents interact with their cognate targets present in the sample. The pattern of binding of targets among all immobilized agents provides a profile of gene expression. Representative microarrays are described, e.g., in U.S. Pat. Nos. 5,412,087, 5,445,934, 5,744,305, 6,897,073, 7,247,469, 7,166,431, 7,060,431, 7,033,754, 6,998,274, 6,942,968, 6,890,764, 6,858,394, 6,770,441, 6,620,584, 6,544,732, 6,429,027, 6,396,995, and 6,355,431.

Disclosed herein are nucleic acid or protein arrays for the detection of at least three of genes or gene products listed in TABLE 2. In particular embodiments, disclosed arrays consist of binding agents specific for at least three, at least four, at least five, at least six, at least seven, at least eight, at least nine, at least 10, at least 11, at least 12, at least 13, at least 14, at least 15, at least 16, at least 17 at least 18 at least 19 at least 20, at least 21, at least 22, at least 23, at least 24, at least 25, at least 26, at least 27 or all 28 of the disclosed genes. In a specific embodiment, an array consists of nucleic acid probes or antibodies specific for ASPM, ATP9A, and ACOX3. In another specific embodiment, an array consists of nucleic acid probes or antibodies specific for ASPM, ATP9A, ACOX3, CDC45L, SLC40A1, and AGR2. In another specific embodiment, an array consists of nucleic acid probes or antibodies specific for ASPM, ATP9A, ACOX3, CDC45L, SLC40A1, AGR2, ATP11C, FAM72A, PLA2G10, MATN2, APITD1, and KIF11. Other array embodiments consist of nucleic acid probes or antibodies specific for each one of the 28 genes listed in TABLE 2, including ASPM, ATP9A, ACOX3, CDC45L, SLC40A1, AGR2, ATP11C, FAM72A, PLA2G10, MATN2, APITD1, KIF11, HPGD, HMMR, ELF3, PTTG1, UPP1, CCNB2, CREG1, ARSD, CENPN, SMC4, DLGAP5, PIK3AP1, TLR3, TWIST1, GCLM and CTSS. In particular examples, the array further includes nucleic acid probes or antibodies specific for one or a plurality of housekeeping genes or gene products, such as mRNA, cDNA or protein.

The nucleic acid probes or antibodies forming the array can be directly linked to the support or attached to the support by oligonucleotides or other molecules that serve as spacers or linkers to the solid support.

The array solid support can be glass slides or formed from an organic polymer. A variety of array formats can be employed in accordance with certain embodiments. For instance, a linear array of oligonucleotide bands, a two-dimensional pattern of discrete cells, or other formats (e.g., U.S. Pat. No. 5,981,185).

A suitable array can be prepared by a variety of approaches. In one example, oligonucleotide or protein sequences are synthesized separately and then attached to a solid support (e.g., U.S. Pat. No. 6,013,789). In another example, sequences are synthesized directly onto the support to provide the desired array (e.g., U.S. Pat. No. 5,554,501). Oligonucleotide probes can be bound to the support by either the 3′ end of the oligonucleotide or by the 5′ end of the oligonucleotide.

### Definitions

As used herein, “pancreatic cancer” refers to malignant mammalian cancers, especially adenocarcinomas, derived from epithelial cells in the exocrine pancreatic tissues. Pancreatic cancers embraced in the current application include both metastatic and non-metastatic cancers.

As used herein, “glandular cancer” refers to malignant tumor originating in glandular epithelium, which includes, but not limited to, exocrine pancreatic glands (pancreatic adenocarcinoma), mammary glands (breast cancer), prostatic glands (prostate cancer), colonic epithelium (colon cancer), gastric epithelium (gastric cancer), salivary glands (salivary gland carcinoma), adrenal glands (adrenal carcinoma), and thyroid glands (thyroid carcinoma).

The term “differentiation” refers to generalized or specialized changes in structures or functions of an organ or tissue during development. The concept of differentiation is well known in the art and requires no further description herein. For example, differentiation of pancreatic cells refers to, among others, the process of glandular structure formation and/or the acquisition of hormonal or secretory functions of normal pancreatic glands.

The term “cancer stem cells” refer to a subpopulation of cancer cells that can self-renew, generate diverse cells in the tumor mass, or initiate a tumor in a host.

As used herein, the term “clinical prognosis” refers to the outcome of subjects with pancreatic cancer comprising the likelihood of tumor recurrence, survival, disease progression, and response to treatments. The recurrence of pancreatic cancer after treatment (e.g., pancreatectomy) is indicative of a more aggressive cancer, a shorter survival of the host (e.g., pancreatic cancer patients), an increased likelihood of an increase in the size, volume or number of tumors, and/or an increased likelihood of failure of treatments.

As used herein, the term “predicting clinical prognosis” refers to providing a prediction of the probable course or outcome of pancreatic cancer, including prediction of metastasis, multidrug resistance, disease free survival, overall survival, recurrence, etc. The methods can also be used to devise a suitable therapy for cancer treatment, e.g., by indicating whether or not the cancer is still at an early stage or if the cancer had advanced to a stage where aggressive therapy would be ineffective.

As used herein, the term “recurrence” refers to the return of a pancreatic cancer after an initial or subsequent treatment(s). Representative treatments include any form of surgery (e.g., pancreaticoduodenectomy or Whipple procedure, distal pancreatectomy, segmental pancreatectomy, and total pancreatectomy), any form of radiation treatment, any form of chemotherapy or biological therapy, any form of hormone treatment. In some examples, recurrence of the pancreatic cancer is marked by rising serum or plasma markers of pancreatic cancer, such as carbohydrate antigen 19-9 (CA19-9) (Koprowski et al., 1981) and carcinoembryonic antigen (CEA) (Gold and Freedman, 1965), and/or by identification of pancreatic cancer cells in any biological sample from a subject with pancreatic cancer.

As used herein, the term “disease progression” refers to a situation wherein one or more indices of pancreatic cancer (e.g, serum CA19-9 or CEA levels, measurable tumor size or volume, or new lesions) show that the disease is advancing despite treatment(s).

“ASPM”, “ATP9A”, “ACOX3”, “CDC45L”, “SLC40A1”, “AGR2” and other molecular markers recited herein, including those found in TABLE 2, refer to nucleic acids, e.g., gene, pre-mRNA, mRNA, and polypeptides, polymorphic variants, alleles, mutants, and interspecies homologs that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of over a region of at least about 25, 50, 100, 200, 500, 1000, or more amino acids, to a polypeptide encoded by a referenced nucleic acid or an amino acid sequence described herein; (2) specifically bind to antibodies, e.g., polyclonal antibodies, raised against an immunogen comprising a referenced amino acid sequence, immunogenic fragments thereof, and conservatively modified variants thereof; (3) specifically hybridize under stringent hybridization conditions to a nucleic acid encoding a referenced amino acid sequence, and conservatively modified variants thereof; (4) have a nucleic acid sequence that has greater than about 60% nucleotide sequence identity, 65%, 70%, 75%, 80%, 85%, 90%, preferably 91%, 92%, 93%, 94%, 95%, 96%, 97%, 98% or 99% or higher nucleotide sequence identity, preferably over a region of at least about 10, 15, 20, 25, 50, 100, 200, 500, 1000, or more nucleotides, to a reference nucleic acid sequence. A polynucleotide or polypeptide sequence is typically from a mammal including, but not limited to, primate, e.g., human; rodent, e.g., rat, mouse, hamster; cow, pig, horse, sheep, or any mammal. The nucleic acids and proteins ofcertain embodiments include both naturally occurring or recombinant molecules. Truncated and alternatively spliced forms of these antigens are included in the definition.

The term “differentially expressed” or “differentially regulated” refers generally to a protein or nucleic acid that is overexpressed (upregulated) or underexpressed (downregulated) in one sample compared to at least one other sample in the context of certain embodiments.

The terms “molecular marker”, “gene marker”, “cancer-associated antigen”, “tumor-specific marker”, “tumor marker”, “marker”, or “biomarker” interchangeably refer to a molecule or a gene (typically protein or nucleic acid such as RNA) that is differentially expressed in the cell, expressed on the surface of a cancer cell or secreted by a cancer cell in comparison to a non-cancer cell or another cancer cells, and which is useful for the diagnosis of cancer, for providing a prognosis, and for preferential targeting of a pharmacological agent to the cancer cell. In certain embodiments, a cancer-associated antigen is a molecule that is overexpressed or underexpressed in a cancer cell in comparison to a non-cancer cell or another cancer cells, for instance, 1-fold over expression, 2-fold overexpression, 3-fold overexpression or more in comparison to a non-cancer cell or, for instance, 20%, 30%, 40%, 50% or more underexpressed in comparison to a non-cancer cell. In certain embodiments, a cancer-associated antigen is a molecule that is inappropriately synthesized in the cancer cell, for instance, a molecule that contains deletions, additions or mutations in comparison to the molecule expressed in a non-cancer cell. In certain embodiments, a cancer-associated antigen will be expressed exclusively on the cell surface of a cancer cell and not synthesized or expressed on the surface of a normal cell. Exemplified cell surface tumor markers include carbohydrate antigen 19-9 (CA19-9) (Koprowski et al., 1981) and carcinoembryonic antigen (CEA) (Gold and Freedman, 1965). In certain embodiments, a cancer-associated antigen will be expressed primarily not on the surface of the cancer cell.

It will be understood by the skilled artisan that markers may be used singly or in combination with other markers for any of the uses, e.g., diagnosis or prognosis of multidrug resistant cancers, disclosed herein.

“Biological sample” includes sections of tissues such as biopsy and autopsy samples, and frozen sections taken for histologic purposes. Such samples include pancreatic cancer tissues, blood and blood fractions or products (e.g., serum, plasma, platelets, red blood cells, and the like), sputum, tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish.

A “biopsy” refers to the process of removing a tissue sample for diagnostic or prognostic evaluation, and to the tissue specimen itself. Any biopsy technique known in the art can be applied to the diagnostic and prognostic methods of certain embodiments. The biopsy technique applied will depend on the tissue type to be evaluated (e.g., pancreas, etc.), the size and type of the tumor, among other factors. Representative biopsy techniques include, but are not limited to, excisional biopsy, incisional biopsy, needle biopsy, surgical biopsy, and bone marrow biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. A diagnosis or prognosis made by endoscopy or fluoroscopy can involve a “core-needle biopsy”, or a “fine-needle aspiration biopsy” which generally obtains a suspension of cells from within a target tissue. Biopsy techniques are discussed, for example, in Harrison's Principles of Internal Medicine, Kasper, et al., eds., 16th ed., 2005, Chapter 70, and throughout Part V.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants” and nucleic acid sequences encoding truncated forms of cancer biomarkers. Similarly, a particular protein encoded by a nucleic acid implicitly encompasses any protein encoded by a splice variant or truncated form of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. Nucleic acids can be truncated at the 5′ end or at the 3′ end. Polypeptides can be truncated at the N-terminal end or the C-terminal end. Truncated versions of nucleic acid or polypeptide sequences can be naturally occurring or recombinantly created.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution table providing functionally similar amino acids are well known in the art. Such conservatively modified variants are in addition to and do not exclude polymorphic variants, interspecies homologs, and alleles of certain embodiments.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Coding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. No. 4,946,778, U.S. Pat. No. 4,816,567) can be adapted to produce antibodies to polypeptides of certain embodiments. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature 321 :522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent complementarity determining regions (CDRs) or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some framework region (FR) residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

In one embodiment, the antibody is conjugated to an “effector” moiety. The effector moiety can be any number of molecules, including labeling moieties such as radioactive labels or fluorescent labels, or can be a therapeutic moiety. In one aspect the antibody modulates the activity of the protein.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions can include an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

### EXAMPLES

The following examples are given for illustrative purposes only and are not intended to be limiting unless otherwise specified. It should be appreciated by those of skill in the art that the techniques disclosed in the examples which follow represent techniques discovered by the inventors to function well in the practice of certain embodiments. Those of skill in the art should appreciate that many changes can be made in the specific embodiments which are disclosed and still obtain a like or similar result without departing from the spirit and scope of the disclosure.

### Example 1

This example describes the identification of the gene expression profile associated with differentiation of pancreatic epithelial tubules.

The tubular differentiation process of prostatic glands was recapitulated by culturing immortalized pancreatic epithelial cells (HPDE cells) within a physiological relevant three-dimensional (3D) culture model, as described before (Weaver et al., 1997). Human pancreatic ductal epithelial (HPDE) cells, which are human papillomavirus-E6 and -E7 gene-immortalized pancreatic ductal epithelial cells (Bello et al., 1997; Liu et al., 1998), were propagated on tissue culture plastics in Keratinocyte-SFM (Sigma-Aldrich, St. Louis, Mo.) supplemented with bovine pituitary extract, 10 ng/ml EGF, 0.5% horse serum and antibiotics (Invitrogen, Carlsbad, Calif.). HPDE cells were seeded on top of a thick layer of 3D reconstituted basement membrane gel (Matrigel, BD Biosciences). A relatively high seeding density of the cells (4×10⁴/cm²) was used to facilitate cell-to-cell interaction and the subsequent tissue morphogenetic process. The culture was maintained in Keratinocyte-SFM (Sigma-Aldrich) supplemented with bovine pituitary extract, 10 ng/ml epidermal growth factor and antibiotics (all from Invitrogen).

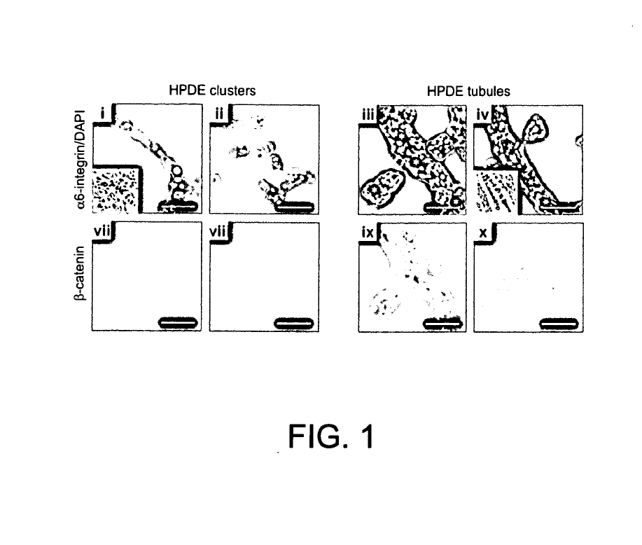

As shown in FIG. 1, when cultured within such a context for a short duration (48 hours), HPDE cells grew into unorganized clusters or cords lacking cell polarization or tissue architectures. Following a prolonged length of time in 3D culture (6-8 days), HPDE cells underwent structural organization, resulting in the formation of branching tubule-like architectures reminiscent of exocrine pancreatic ducts or the tubular structures seen in low-grade PDAC. Confocal imaging analysis revealed that these tubules consisted of a single layer of polarized cells, indicated by the polarized expressions of the basal surface marker α6-integrin and the adherens junction protein β-catenin, and a cell-free lumen.

To dissect the gene expression alterations related to this pancreatic tubular differentiation process, global gene expression profiling experiments was carried out on HPDE cells clusters formed in early-stage culture and tubules formed at latter stages. Briefly, total RNA samples were extracted using TRIZOL (Invitrogen) and then purified using a RNeasy mini-kit and a DNase treatment (Qiagen). Experiments were performed in triplicate. Gene expression analysis was performed on an Affymetrix Human Genome U133A 2.0 Plus GeneChip platform according to the manufacturer's protocol (Affymetrix). The hybridization intensity data was processed using the GeneChip Operating software (Affymetrix) and the genes were filtered based on the Affymetrix P/A/M flags to retain the genes that were present in at least three of the replicate samples in at least one of the culture conditions. A filtering criterion (P<0.01 by Student's t test, fold-change>2.0X) was used to select differentially expressed genes within a comparison group.

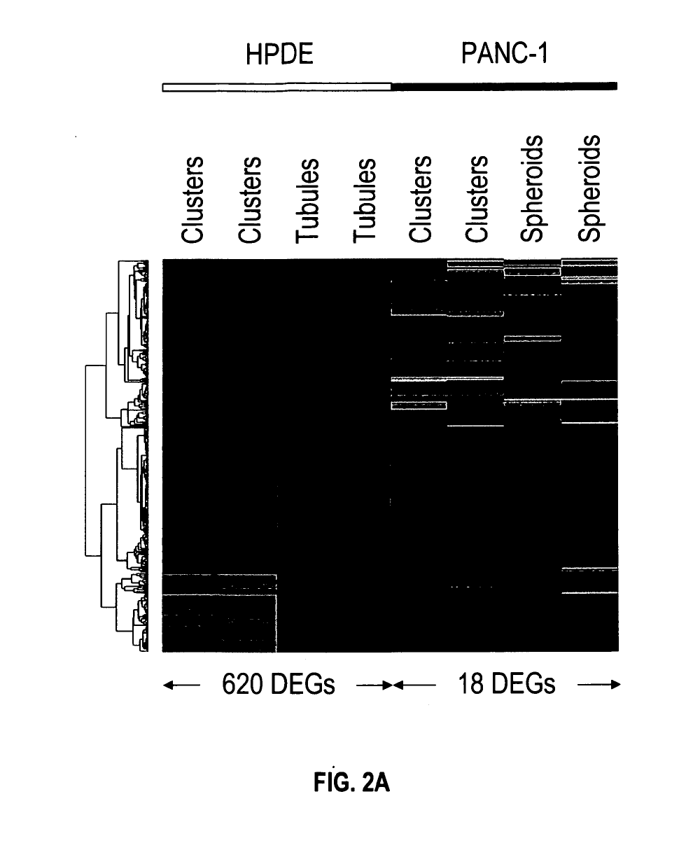

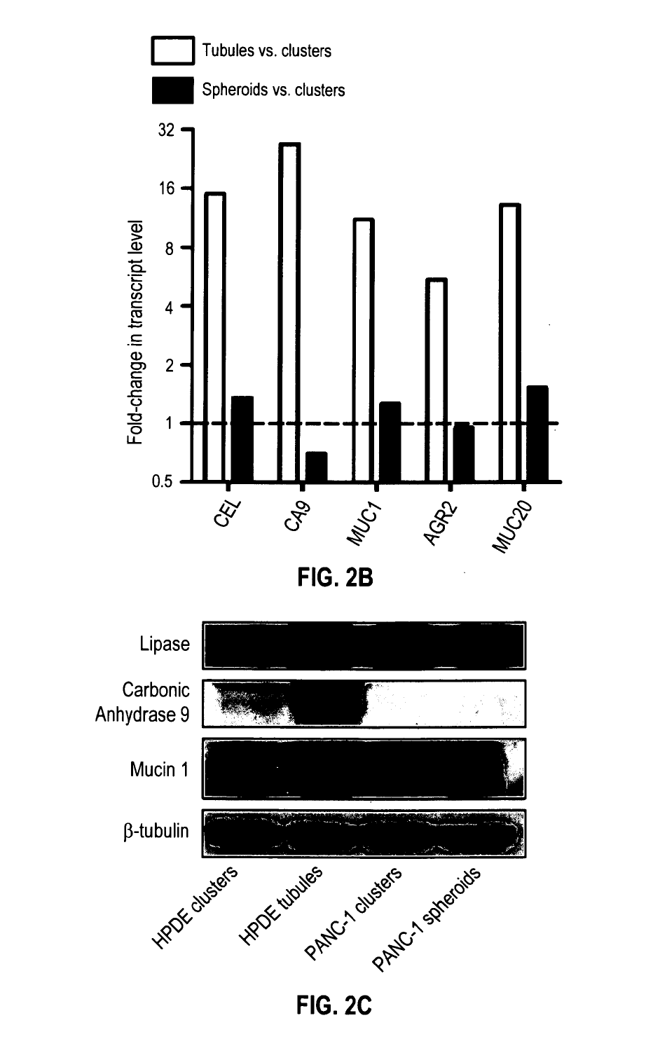

As shown in FIG. 2A, a list of 620 unique genes whose transcript levels varied significantly during tubular morphogenesis of HPDE cells was identified through the gene expression profiling experiments. As a comparison, only a few (18 genes) genes were found to be differentially expressed during the formation of PANC-1 tumor cell spheroids.

As shown in FIG. 2B, several genes that specify the exocrine functions of pancreas, including CEL (bile salt-stimulated lipase), CA9 (carbonic anhydrase 9), MUC1 (mucin 1), AGR2 (anterior gradient homolog 2), and MUC20 (mucin 20), were profoundly up-regulated (up to 26.9-fold) during tubular morphogenesis (white columns), whereas their expressions remained unaltered during the formation of tumor spheroids. Immunoblotting analysis confirmed the tubulogenesis-specific expressional changes in these pancreatic functional markers (FIG. 2C). These findings lend strong supports to our tissue organization model as a valid way to capture the molecular signals specific to the structural and functional differentiation processes of exocrine pancreatic epithelium.

### Example 2

This example describes the identification of a 28-gene prognostic model of pancreatic cancer based on the molecular profile related to pancreatic tubular differentiation.

As disruption of tissue microarchitectures is one of the hallmark features of glandular cancers including PDAC (Adsay et al., 2005; Gleason, 1992; Rakha et al., 2008; Stamey et al., 1999), we investigated whether the gene expression profile associated with pancreatic epithelial tubulogenesis may carry prognostic information in PDAC. We mapped the 620 tubulogenesis-related genes to the UCSF data set) (Collisson et al., 2011) and constructed a “risk score” based on a Cox's model to predict overall survival of the patients. We used a previously described supervised approach with modifications (Wang et al., 2005). Briefly, for each gene, univariate Cox's regression analysis was used to measure the correlation between the expression level of the gene (on a log₂ scale) and the length of survival of the patients. We constructed 1000 bootstrap samples of the patients in the cohort and performed Cox's regression analysis on each of the samples. We then determined an estimated P-value and an estimated standardized Cox regression coefficient for each gene by calculating the median P-values and the median Cox's coefficient of the 1000 bootstrap samples, respectively. The selected genes were then ranked-ordered according to the estimated P-values, and multiple sets of genes were generated by repeatedly adding one more genes each time from top of the descendingly ranked list, starting from the first three top-ranked genes. We then calculated a “risk score” (Equation 1) to measure the risk of death of a patient for a gene set:

Risk score=Σᵢ₌₃^{k}bᵢxᵢ (Equation 1)

where k is the number of probes in the probe set, bᵢ is the standardized Cox regression coefficient for the ith probe and xᵢ is the log₂ expression level for the ith probe.

For each selected probe set the concordance index (C-index) was used to evaluate the predictive accuracy in survival analysis (Pencina and D'Agostino, 2004). C-index statistics analysis was conducted using the ‘survcomp’ package in the statistical programming language R (cran.r-project.org). The gene set that achieved the maximal predictive accuracy while contained the fewest number of the genes was selected as the optimized prognostic predictor.

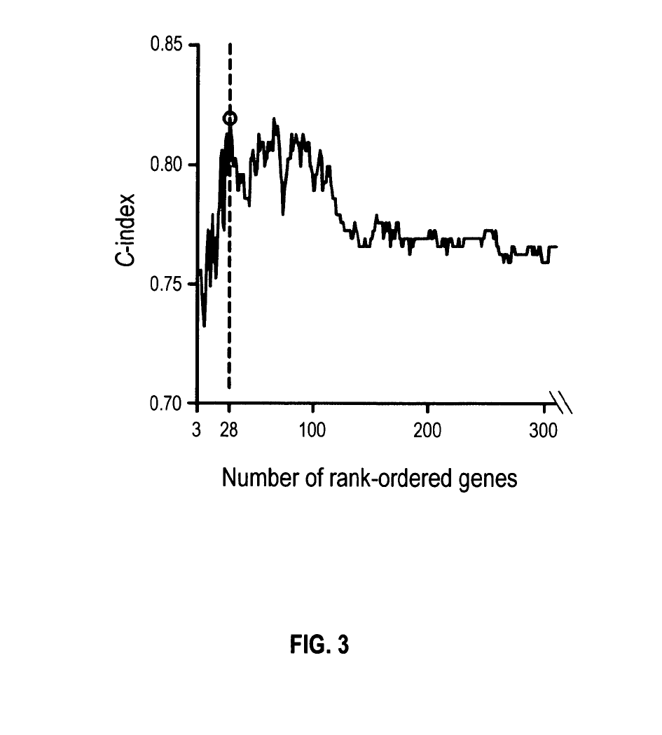

As shown in FIG. 3, through this approach, we selected a set of 28 genes whose performance in the prognostic prediction, as assessed by C-index, reached a plateau.

TABLE 2 shows the identities of the 28 selected genes.

TABLE 2
Description of genes in the 28-gene signature

| Cox | | | |
|---|---|---|---|
| regression | | Entrez | |
| P value | Symbol | gene ID | Gene title |
| 0.0026 | ATP9A | 10079 | ATPase, class II, type 9A |
| 0.0028 | ASPM | 259266 | Asp (abnormal spindle) homolog, |
| | | | microcephaly associated |
| 0.0032 | ACOX3 | 8310 | Acyl-Coenzyme A oxidase 3, pristanoyl |
| 0.0032 | CDC45L | 8318 | CDC45 cell division cycle 45-like |
| 0.0043 | SLC40A1 | 30061 | Solute carrier family 40 (iron-regulated |
| | | | transporter), member 1 |
| 0.0049 | AGR2 | 10551 | Anterior gradient homolog 2 |
| 0.0065 | ATP11C | 286410 | ATPase, class VI, type 11C |
| 0.0069 | FAM72A | 653573 | Family with sequence similarity 72, |
| | | | member A |
| 0.0083 | PLA2G10 | 8399 | Phospholipase A2, group X |
| 0.0088 | MATN2 | 4147 | Matrilin 2 |
| 0.0095 | APITD1 | 378708 | Apoptosis-inducing, TAF9-like domain 1 |
| 0.0100 | KIF11 | 3832 | Kinesin family member 11 |
| 0.0108 | HPGD | 3248 | Hydroxyprostaglandin dehydrogenase |
| | | | 15-(NAD) |
| 0.0114 | HMMR | 3161 | Hyaluronan-mediated motility receptor |
| | | | (RHAMM) |
| 0.0119 | ELF3 | 1999 | E74-like factor 3 (ets domain |
| | | | transcription factor, epithelial-specific) |
| 0.0135 | PTTG1 | 9232 | Pituitary tumor-transforming 1 |
| 0.0149 | UPP1 | 7378 | Uridine phosphorylase 1 |
| 0.0161 | CCNB2 | 9133 | Cyclin B2 |
| 0.0172 | CREG1 | 8804 | Cellular repressor of E1A-stimulated |
| | | | genes 1 |
| 0.0172 | ARSD | 414 | Arylsulfatase D |
| 0.0174 | CENPN | 55839 | Centromere protein N |
| 0.0190 | SMC4 | 10051 | Structural maintenance of |
| | | | chromosomes 4 |
| 0.0192 | DLGAP5 | 9787 | Discs, large homolog-associated |
| | | | protein 5 |
| 0.0193 | PIK3AP1 | 118788 | Phosphoinositide-3-kinase adaptor |
| | | | protein 1 |
| 0.0200 | TLR3 | 7098 | Toll-like receptor 3 |
| 0.0201 | TWIST1 | 7291 | Twist homolog 1 |
| 0.0205 | GCLM | 2730 | Glutamate-cysteine ligase, modifier |
| | | | subunit |
| 0.0208 | CTSS | 1520 | Cathepsin S |

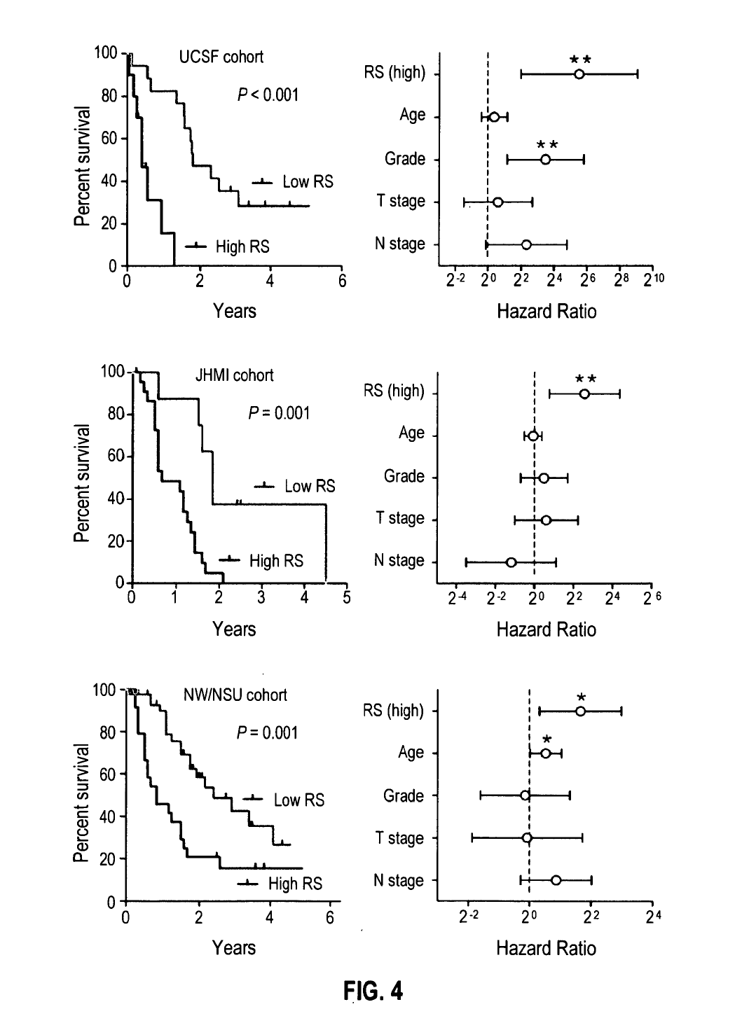

FIG. 4 shows that, based on the risk score (Equation 1), the expression profile of this 28 gene signature could very effectively stratify risk of death by Kaplan-Meier analysis in three independent cohorts of patients with PDAC, including the UCSF cohort, the JHMI cohort, and the NW/NSF cohort (log-rank test P≦0.001). For example, in the UCSF data set, patients in the high risk group had poor post-operative prognosis with a medium overall survival of 4.9 months, whereas patients in the low-risk group fared well with a medium overall survival of 21.6 months. FIG. 4 also shows that, according to multivariate Cox proportional-hazards analyses, this 28-gene signature was the strongest prognostic predictor of survival in these cohorts of patients with PDAC and its prediction significantly outperformed clinical and pathological criteria, including age, the pathologic grade of tumor, and the tumor stage or lymph node status.

As shown in TABLE 3, multivariate Cox regression analysis demonstrates that this 28-gene model provides strong and independent prognostic information to PDAC in three independent clinical data sets.

TABLE 3
Multivariate Cox regression model predicting overall survival by the
28-gene-based risk score and clinico-pathological criteria

| Variables | Hazard ratio | 95% CI | P-value |
|---|---|---|---|
| UCSF cohort | | | |
| Patient age | 1.305 | 0.751-2.269 | 0.345 |
| (per 10 years) | | | |
| Tumor grade (3 vs. <3) | 11.212 | 2.254-55.771 | 0.003* |
| T stage (3 vs. <3) | 1.528 | 0.359-6.511 | 0.566 |
| N stage (1 vs. 0) | 5.065 | 0.927-27.681 | 0.061 |
| Risk score | 45.422 | 3.924-525.749 | 0.002* |
| (high vs. low)** | | | |
| JHMI cohort | | | |
| Patient age | 0.959 | 0.702-1.31 | 0.792 |
| (per 10 years) | | | |
| Tumor grade (3 vs. <3) | 1.410 | 0.606-3.28 | 0.425 |
| T stage (3 vs. <3) | 1.522 | 0.492-4.715 | 0.466 |
| N stage (1 vs. 0) | 0.439 | 0.089-2.169 | 0.313 |
| Risk score | 5.919 | 1.702-20.578 | 0.005* |
| (high vs. low)** | | | |
| NW/NSU cohort | | | |
| Patient age | 1.446 | 1.015-2.058 | 0.041 |
| (per 10 years) | | | |
| Tumor grade (3 vs. <3) | 0.907 | 0.33-2.492 | 0.850 |
| T stage (3 vs. <3) | 0.948 | 0.273-3.298 | 0.934 |
| N stage (1 vs. 0) | 1.819 | 0.815-4.058 | 0.144 |
| Risk score | 3.159 | 1.255-7.952 | 0.015* |
| (high vs. low)** | | | |

CI: confidence interval.
*P < 0.05
**The threshold was determined by the maximal Youden's index.

TABLE 4 shows that the 28-gene model markedly enhanced the prognostic accuracy of a combined clinical model including clinical and pathological variables (P=0.000-0.011) and outperformed several previously reported prognostic gene signatures of PDAC, including the 62-gene “PDAssigner” and the 6-gene “metastasis signature” in three independent data sets (Collisson et al., 2011; Stratford et al., 2010).

TABLE 4
The prediction accuracy, as evaluated by C-index, of different
prognosis prediction models in three independent cohorts of patients
with PDAC

| | C-index | 95% CI | P-value |
|---|---|---|---|
| UCSF cohort | | | |
| Clinico-pathological criteria* | 0.802 | 0.720-0.884 | |
| Risk score by 28-gene model | 0.899 | 0.783-1.000 | 0.000 |
| Risk score by PDAssigner* * | 0.752 | 0.644-0.861 | 0.434 |
| Risk score by metastasis | 0.679 | 0.536-0.822 | 0.988 |
| signature* * * | | | |
| JHMI cohort | | | |
| Clinico-pathological criteria* | 0.574 | 0.467-0.681 | |
| Risk score by 28-gene model | 0.890 | 0.767-1 | 0.000 |
| Risk score by PDAssigner* * | 0.586 | 0.448-0.724 | 0.431 |
| Risk score by metastasis | 0.684 | 0.569-0.798 | 0.084 |
| signature* * * | | | |
| NW/NSU cohort | | | |
| Clinico-pathological criteria* | 0.672 | 0.574-0.771 | |
| Risk score by 28-gene model | 0.800 | 0.682-0.919 | 0.011 |
| Risk score by PDAssigner* * | 0.649 | 0.556-0.742 | 0.576 |
| Risk score by metastasis | 0.626 | 0.538-0.715 | 0.671 |
| signature* * * | | | |

C-index: concordance index; CI: confidence interval.
*Clinico-pathological criteria include age, tumor grade, T stage and N stage status.
* *The reported molecular subtypes of PDAC were defined by a 62-gene signature, “PDAssigner”; Collisson EA, et al. Nat. Med. 2011; 17: 500-503.
* * *The six-gene metastasis signature includes FBJ murine osteosarcoma viral oncogene homolog B (FOSB), Kruppel-like factor 6 (KLF6), nuclear factor of kappa light polypeptide gene enhancer in B-cells inhibitor, zeta (NFKBIZ), ATPase H+/K+ exchanging, alpha polypeptide (ATP4A), germ cell associated 1 (GSG1), and sialic acid binding Ig-like lectin 11 (SIGLEC11); Stratford JK, et al. PLoS Med. 2010; 7(7): e1000307.

### Example 3

This example describes the prognostic value of ASPM and selected markers listed in TABLE in human PDAC.

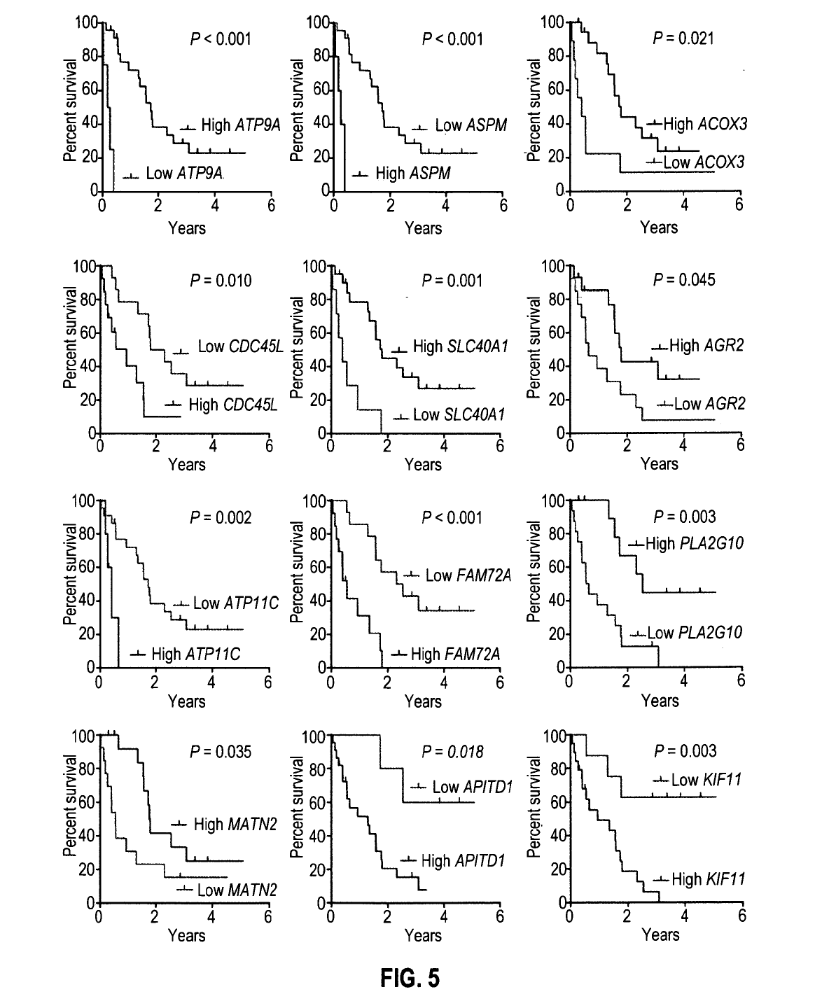

FIG. 5 shows that the top 12 selected gene markers listed in TABLE 2 could individually stratify PDAC patients into two groups that exhibited significant difference in risk for death following surgery (P=0.001-0.045 by log-rank test).

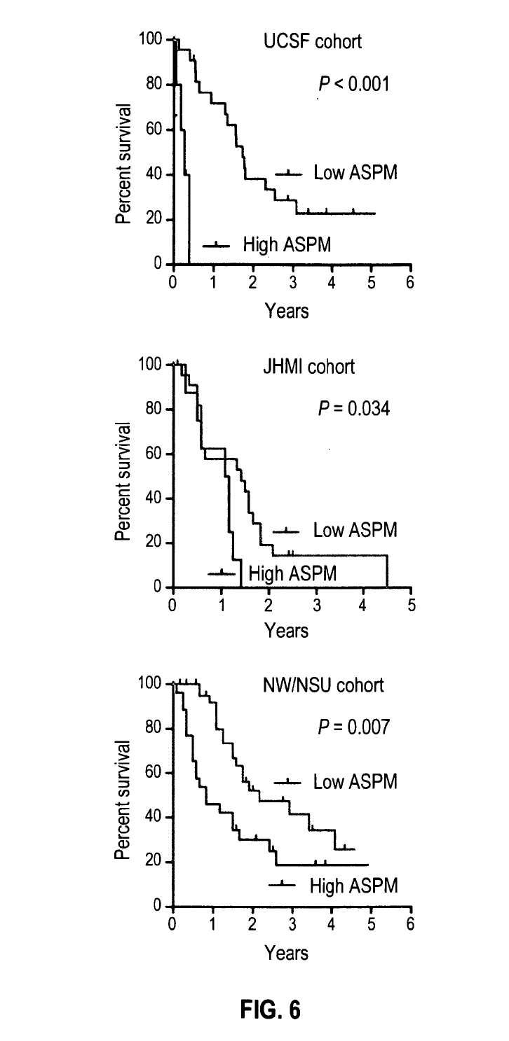

Among the constituent genes in the 28 gene signature in TABLE, the gene ASPM exhibited the most prominent transcriptional change during pancreatic tubular differentiation. We correlated the expression of ASPM with clinical outcome in patients with PDAC and found that patients with their tumors expressing high transcript levels of ASPM fared poorly across three independent patient cohorts (P=0.001-0.034 by log-rank test; FIG. 6).

### Example 4

This example describes the role of ASPM in pancreatic cancer progression.

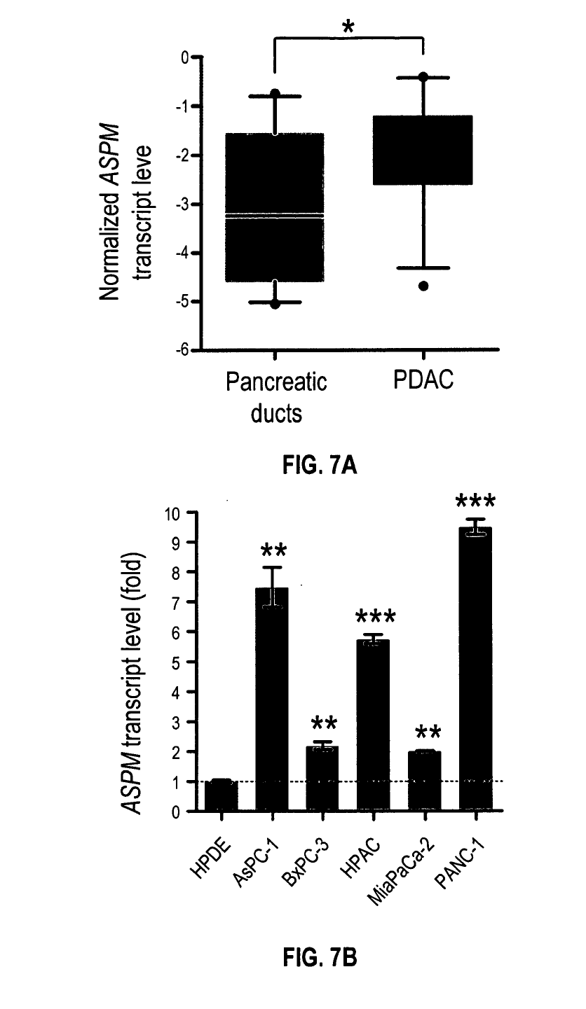

To assess if ASPM plays a role in pancreatic tumorigenesis, we conducted Oncomine expression analysis (https://www.oncomine.com/resource/login.html) and found that the transcript level of ASPM significantly increased in human PDAC tissues relative to normal pancreatic ducts (FIG. 7A) (Grutzmann et al., 2004). We surveyed the transcript level of ASPM in a panel of pancreatic epithelial cell lines, including AsPC-1, BxPC-3, HPDA, MiaPaCa-2, and PANC-1 cells. ASPM expression was up-regulated in most PDAC cells relative to HPDE cells (FIG. 7B).

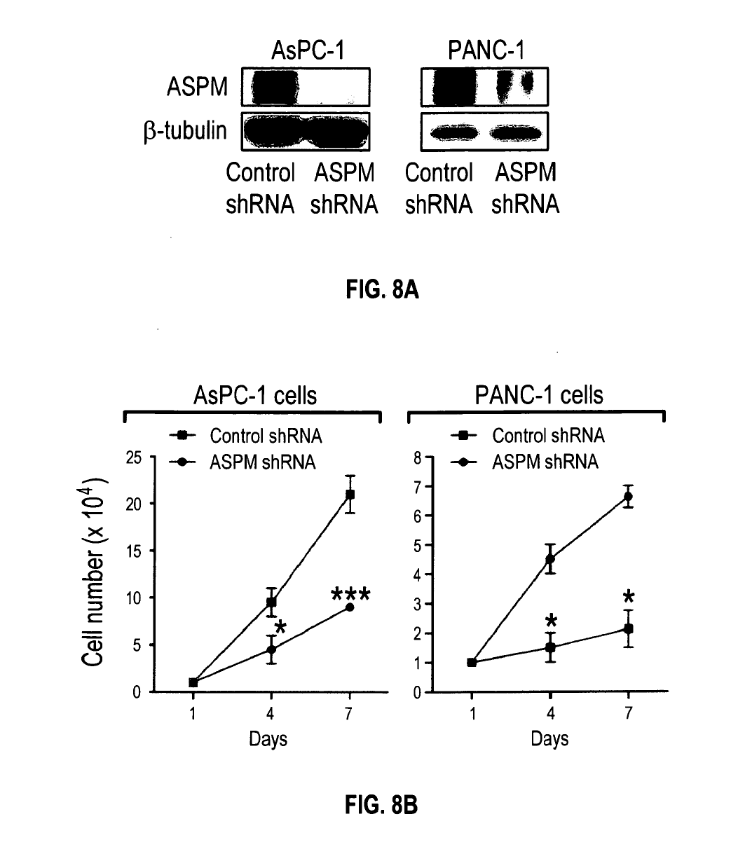

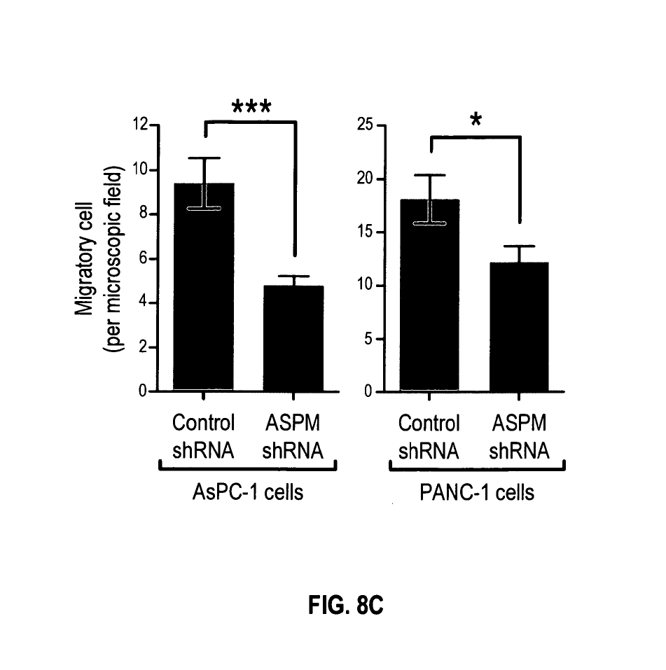

To further investigate the biological function of ASPM in PDAC cells, we stably down-regulated its expression in PDAC cells by using lentivirus-mediated RNA interference (RNAi). Sustained ASPM knockdown was achieved by using validated short hairpin RNA (shRNA) oligonucleotides in the lentivector pLKO.1-puro (MISSION shRNA lentiviruses; Sigma-Aldrich, St. Louis, Mo.) according the manufacturer's protocol. The clones selected were: ASPM (TRCN0000118905 and TRCN0000291125), and non-target control (SHC002V). FIG. 8A shows the level of ASPM knockdown as verified by immunoblot analysis.

As shown in FIG. 8B, knockdown of endogenous ASPM expression in metastatic AsPC-1 cells or primary tumor-derived PANC-1 cells could respectively attenuate cellular proliferation.

To access the effect of ASPM silencing on the migrative capacity of PDAC cells, we conducted modified Boyden chamber assay. Briefly, cells were seeded on Transwell inserts (Corning, Tewksbury, Mass.) with PSCs seeded in the lower compartments of the Transwell. After an incubation period of 24 hours, the cells that invaded the filters were fixed and stained with DAPI. Migrated cells were counted using a fluorescence microscope. As shown in FIG. 8C, silencing of ASPM expression significantly attenuated cellular migration of AsPC-1 and PANC-1 cells in response to pancreatic stellate cells.

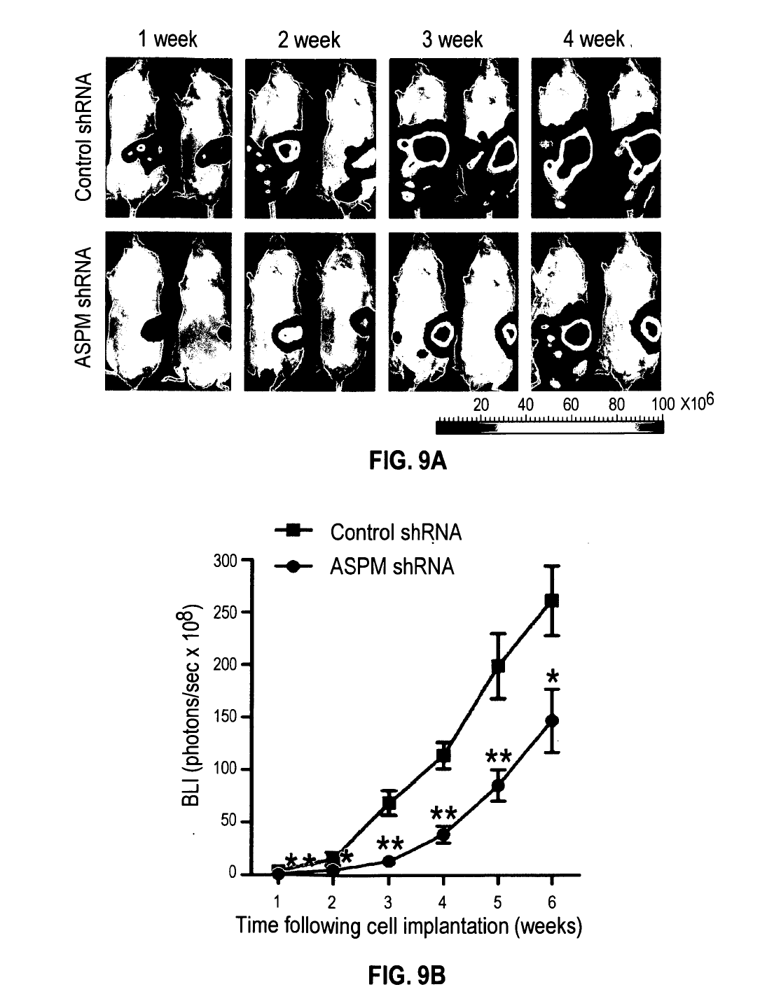

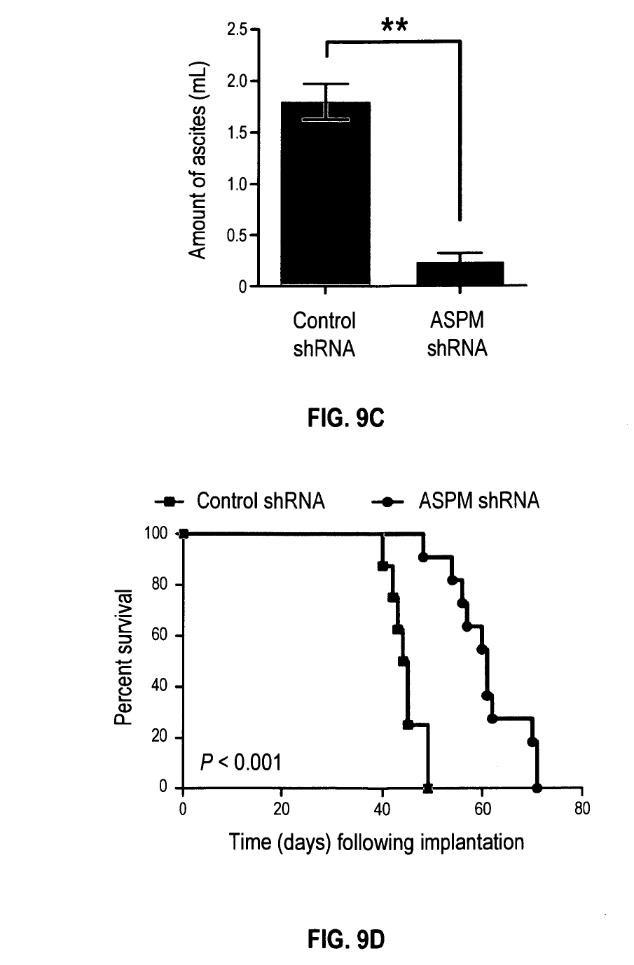

The role of ASPM in PDAC cellular growth and migration raised the possibility that it may contribute to PDAC progression in vivo. To address this possibility, we stably expressed a firefly luciferase reporter in control or ASPM shRNA-transduced AsPC-1 cells and ortho-topically implanted them into the pancreatic tail of NOD-SCID mice. Indeed, the tumors expressing the ASPM shRNA grew significantly slower than the control tumors, reaching approximately one-third the size of the control tumors 4 weeks following transplantation (FIG. 9A).

As shown in FIG. 9B, compared with mice harboring control tumors, animals with ASPM-deficient tumors exhibited significantly prolonged survival so that these animals survived on average 37% (16.5 days) longer than the control animals (log-rank test P<0.001).

### Example 5

This example describes the role of ASPM in Wnt signaling pathway and β-catenin protein stability in PDAC cells.

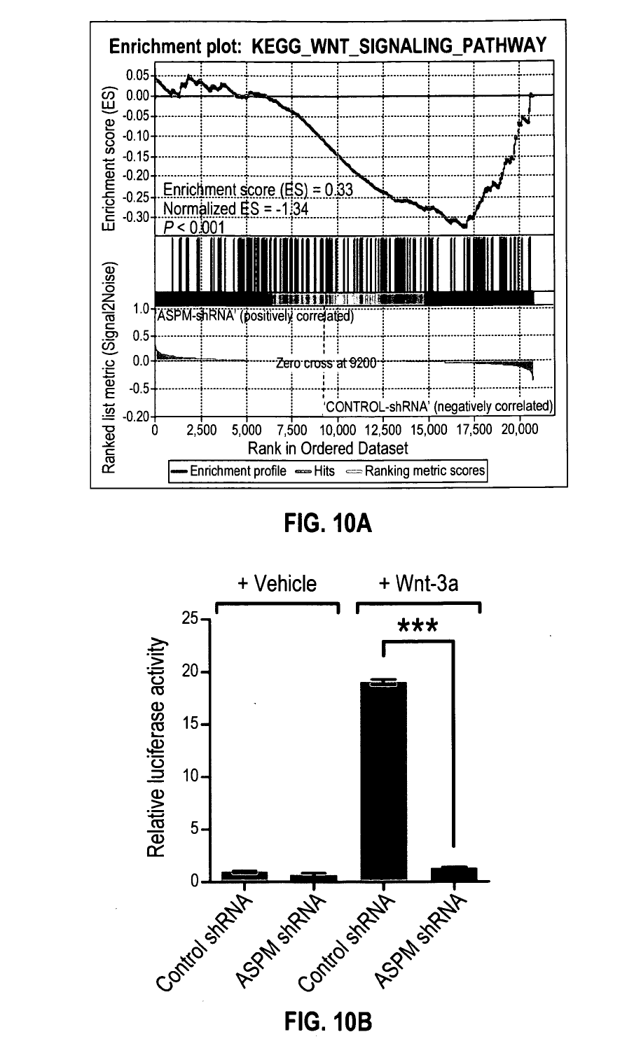

To gain insight into the molecular mechanisms underlying the oncogenic role of ASPM in PDAC, we compared the transcriptomes of control and ASPM shRNA-transduced AsPC-1 cells. We surveyed the whole gene expression profile by using Gene Set Enrichment Analysis (GSEA) and found that the KEGG Wnt signaling pathway was among the gene sets significantly enriched (P<0.001) in the differential gene expression profile (FIG. 10A).

To assess if ASPM regulates Wnt pathway activity, we expressed a Wnt reporter construct in AsPC-1 cells. Cells were transduced with Cignal Lenti TCF/LEF Reporter (Qiagen) according to the manufacturer's protocol. Following stimulation of the cells with recombinant human Wnt-3a (250 ng/mL for 16 hours; R&D Systems, Minneapolis, Minn.) or vehicle the reporter activity was measured by using the ONE-Glo™ Luciferase Assay System (Promega, Madison, Wis.).

FIG. 10B shows that, when the Wnt signaling was activated in AsPC-1 cells by the canonical Wnt ligand Wnt-3a, cells depleted with ASPM exhibited dramatically blunted Wnt-mediated luciferase reporter activation. This result confirmed that ASPM is functionally important for the Wnt signaling pathway activity in PDAC cells.

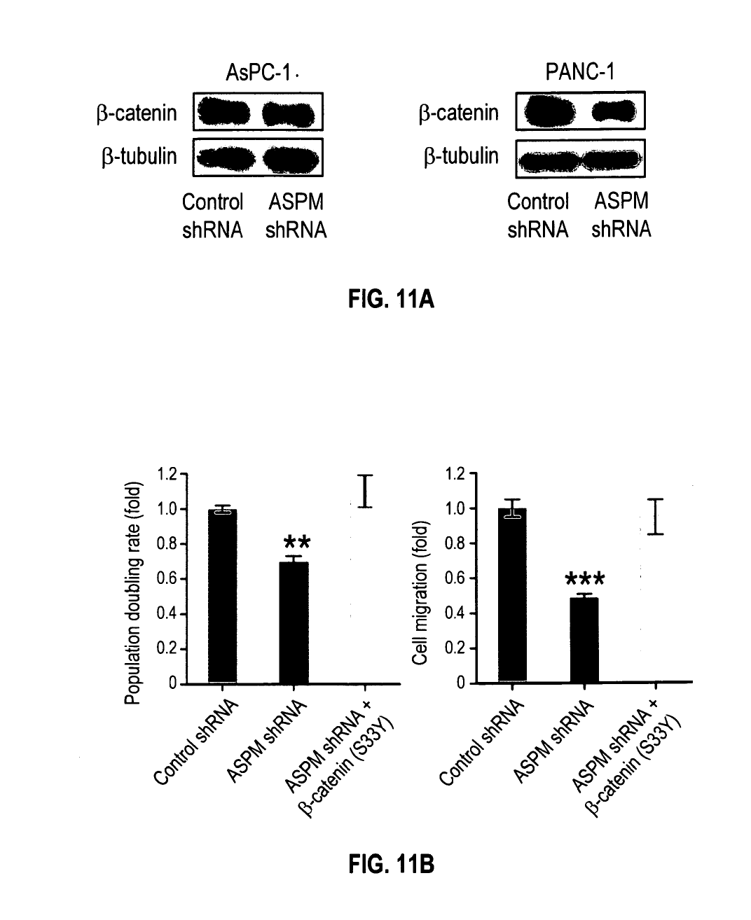

β-catenin is an essential downstream mediator of Wnt signaling pathway and its active form frequently accumulates in PDAC tissues and contributes to PDAC maintenance (Pasca di Magliano et al., 2007; Wang et al., 2009). To assess if ASPM modulates Wnt signaling pathway activity by regulating β-catenin, β-catenin expression was probed in control or ASPM shRNA-transduced cells by Western blot analysis. FIG. 11A shows that silencing of ASPM expression resulted in a decrease in the expression of β-catenin in both AsPC-1 and PANC-1 cells.

To further address if down-regulation of β-catenin mediated the cellular effects induced by ASPM silencing, we stably expressed a constitutively active S33Y mutant of β-catenin in ASPM shRNA-transduced AsPC-1 cells (Kolligs et al., 1999). Indeed, functional activation of β-catenin in ASPM-deficient cells could restore their proliferative as well as migratory potentials (FIG. 11B).

### Example 6

This example describes the role of ASPM in pancreatic cancer stem cells.

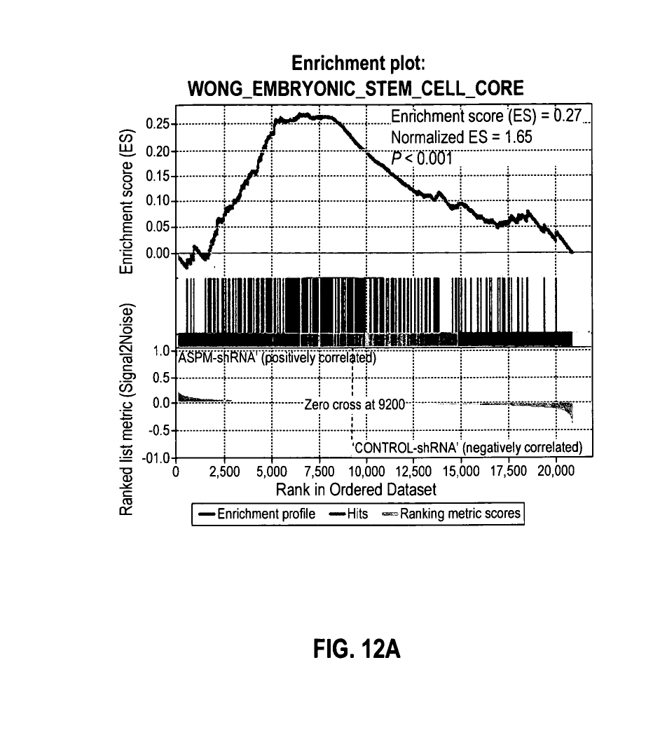

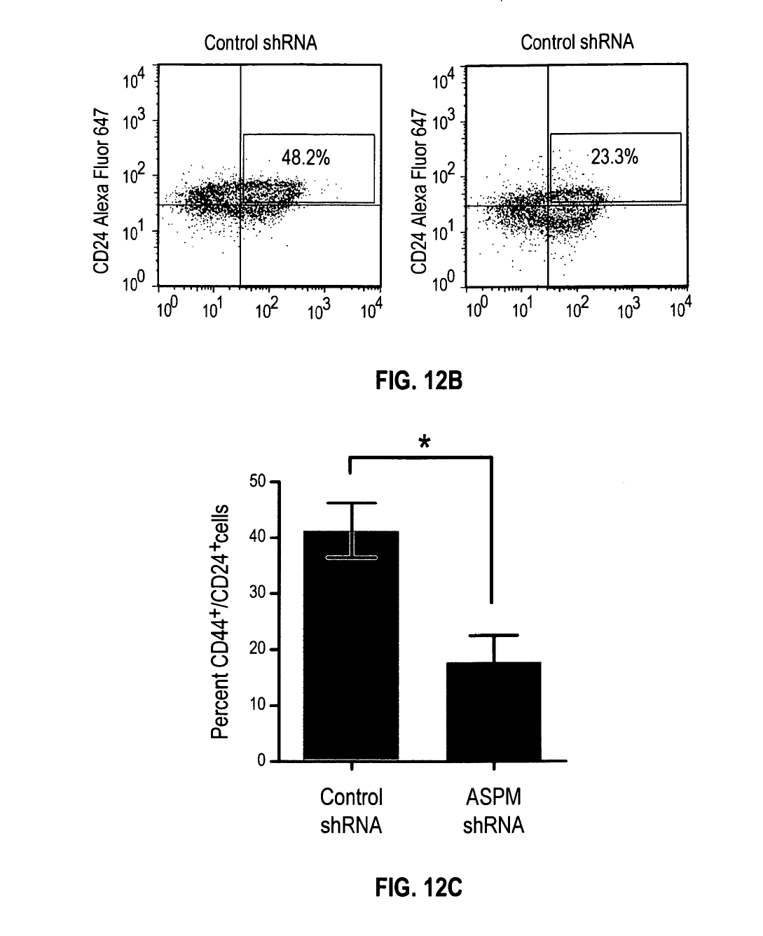

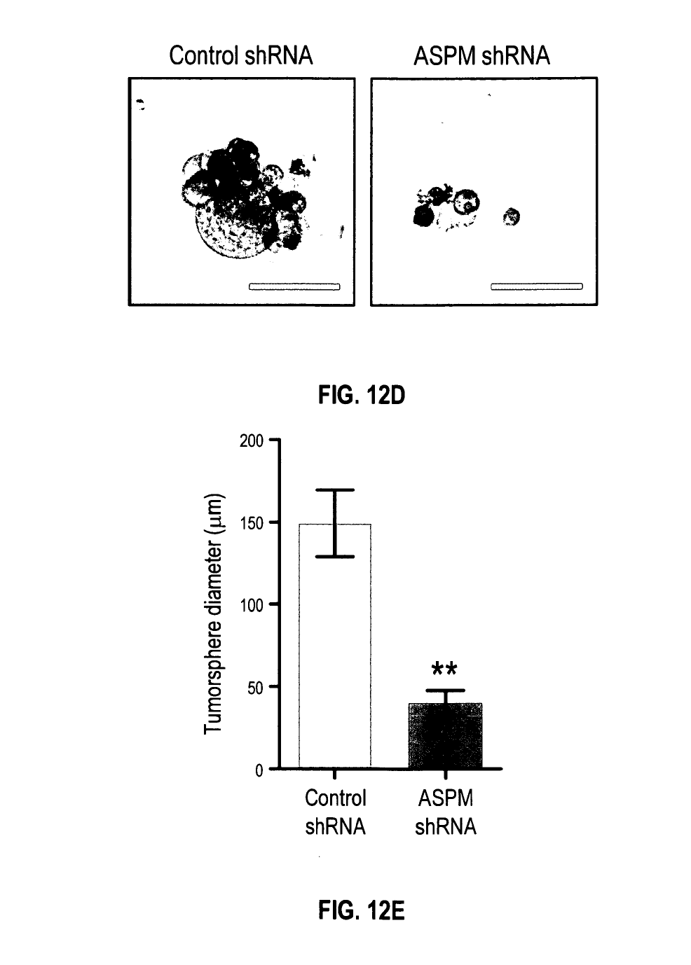

Previously, studies have indicated a role of ASPM in regulating neural stem cells (Buchman et al., 2011; Horvath et al., 2006). Consistently, we found that a core stem cells-like gene module that is activated in human cancers was significantly enriched by Gene Set Enrichment Analysis (P<0.001) in the gene profile associated with silencing ASPM (FIG. 12A) (Wong et al., 2008). This finding, together with the reported role of Wnt signaling in stem-like cells in gastrointestinal malignancies (Pasca di Magliano et al., 2007; Vermeulen et al., 2010), prompted us to investigate if ASPM regulates pancreatic cancer stem cells.

We measured the proportion of cells that co-expressed CD44 and CD24, which contains the enriched cancer stem-like cells in PDAC (Li et al., 2007), in control or ASPM shRNA-transduced AsPC-1 cells. Cells were dissociated, antibody-labeled and resuspended in HBSS/2% FBS containing DAPI as previously described (Li et al., 2007). The antibodies used included PE anti-CD44, and Alexa Fluor 647 anti-CD24 (BD Biosciences). Flow cytometry was done using a FACSCanto II flow cytometer (BD Biosciences).

As shown in FIG. 12B and FIG. 12C, knockdown of ASPM led to a substantial reduction (57.1%) of the CD44⁺CD24⁺ tumor cell population. The ability of ASPM to maintain cancer stemness provides an additional mechanistic explanation for its oncogenic role in PDAC.

To explore the functional relevance of the above finding, we preformed tumorsphere assay on flow-sorted CD44⁺CD24⁺ AsPC-1 cells as previously described (Arensman et al., 2013). Cells were sorted by FACS (BD FACSAria™ III cell sorter; BD Biosciences) and tumorspheres were maintained on ultra-low adherent plates (Corning Inc., Lowell, Mass., USA) in Neurobasal Media according to the manufacturer's instructions (Invitrogen). Equal numbers of live cells were plated in ultralow attachment plates to generate the primary spheres. After 7 days, the mammospheres's sizes were measured and pictures were taken. FIG. 12D and FIG. 12E shows that knockdown of ASPM substantially reduced the growth and the size of the tumorspheres. Together, these data suggests that ASPM is an important regulator of pancreatic cancer stemness.

### Example 7

This example describes a 12-gene prognostic model of PDAC based on the expression levels of ATP9A, ASPM, ACOX3, CDC45L, SLC40A1, AGR2, ATP11C, FAM72A, PLA2G10, MATN2, APITD1, and KIF11.

We sought to condense the prognostic signature of PDAC and investigated if we could use the expression levels of the 12 top-ranked genes in TABLE, including ATP9A, ASPM, ACOX3, CDC45L, SLC40A1, AGR2, ATP11C, FAM72A, PLA2G10, MATN2, APITD1, and KIF11 (Cox regression P value≦0.01), to establish an effective prognostic model of PDAC. We calculated the risk score (Equation 1) based on the transcript levels of these genes in independent cohorts of patients with PDAC, including the UCSF cohort, the JHMI cohort, and the NW/NSF cohort. The patients were stratified into high-risk or low-risk group according to the risk score with the threshold determined by the maximal Youden's index.

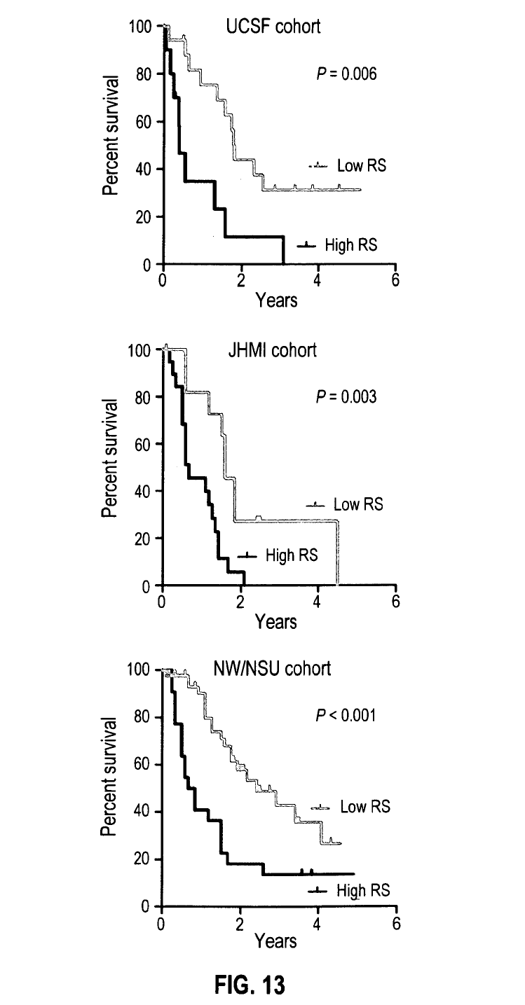

As shown in FIG. 13, based on the risk score, the transcript levels of ATP9A, ASPM, ACOX3, CDC45L, SLC40A1, AGR2, ATP11C, FAM72A, PLA2G10, MATN2, APITD1, and KIF11 could very effectively stratify risk of death by Kaplan-Meier analysis in each of the patient cohorts (log-rank test P=0.001-0.006).

As shown in TABLE 5, multivariate Cox regression analysis demonstrates that this 12-gene model provides strong prognostic information to PDAC independent of clinical and pathological criteria in each of the three PDAC data sets.

TABLE 5
Multivariate Cox regression model predicting overall survival by the
12-gene model and clinico-pathological criteria in independent cohorts
of patients with PDAC

| Variables | Hazard ratio | 95% CI | P-value |
|---|---|---|---|
| UCSF cohort | | | |
| Patient age | 1.374 | 0.831-2.274 | 0.220 |
| (per 10 years) | | | |
| Tumor grade (3 vs. <3) | 4.554 | 1.234-16.8 | 0.023* |
| T stage (3 vs. <3) | 1.232 | 0.308-4.927 | 0.770 |
| N stage (1 vs. 0) | 11.023 | 1.666-72.926 | 0.013* |
| Risk score by 12-gene | 6.398 | 1.465-27.936 | 0.014* |
| model (high vs. low)** | | | |
| JHMI data set | | | |
| Patient age | 1.037 | 0.713-1.508 | 0.848 |
| (per 10 years) | | | |
| Tumor grade (3 vs. <3) | 1.734 | 0.719-4.181 | 0.220 |
| T stage (3 vs. <3) | 1.586 | 0.464-5.421 | 0.462 |
| N stage (1 vs. 0) | 0.370 | 0.067-2.03 | 0.252 |
| Risk score by 12-gene | 4.842 | 1.45-16.163 | 0.010* |
| model (high vs. low)** | | | |
| NW/NSU cohort | | | |
| Patient age | 1.428 | 0.998-2.044 | 0.052 |
| (per 10 years) | | | |
| Tumor grade (3 vs. <3) | 1.009 | 0.392-2.595 | 0.985 |
| T stage (3 vs. <3) | 0.882 | 0.264-2.943 | 0.838 |
| N stage (1 vs. 0) | 1.936 | 0.867-4.324 | 0.107 |
| Risk score by 12-gene | 2.980 | 1.263-7.032 | 0.013* |
| model (high vs. low)** | | | |

CI, confidence interval.
*P < 0.05
**The threshold was determined by the maximal Youden's index.

TABLE 6 shows that, according to C-index values, the predictive accuracy of the 12-gene model outperformed a combined clinical model and several previously reported prognostic gene signatures of PDAC in three independent data sets.

TABLE 6
The prediction accuracy, as evaluated by C-index, of the 12-gene
model and different prognosis prediction models in independent cohorts
of patients with PDAC

| | C-index | 95% CI | P-value |
|---|---|---|---|
| UCSF cohort | | | |
| Clinico-pathological criteria* | 0.802 | 0.720-0.884 | |
| Risk score by 12-gene model | 0.894 | 0.751-1.000 | 0.090 |
| Risk score by PDAssigner* * | 0.805 | 0.692-0.919 | 0.477 |
| Risk score by metastasis | 0.573 | 0.402-0.744 | 0.993 |
| signature* * * | | | |
| JHMI cohort | | | |
| Clinico-pathological criteria* | 0.574 | 0.491-0.656 | |
| Risk score by 12-gene model | 0.832 | 0.698-0.967 | 0.001 |
| Risk score by PDAssigner* * | 0.586 | 0.448-0.724 | 0.431 |
| Risk score by metastasis | 0.684 | 0.569-0.798 | 0.084 |
| signature* * * | | | |
| NW/NSU cohort | | | |
| Clinico-pathological criteria* | 0.672 | 0.574-0.771 | |
| Risk score by 12-gene model | 0.824 | 0.695-0.954 | 0.018 |
| Risk score by PDAssigner* * | 0.686 | 0.588-0.784 | 0.410 |
| Risk score by metastasis | 0.640 | 0.538-0.743 | 0.678 |
| signature* * * | | | |

C-index, concordance index; CI, confidence interval.
*Clinico-pathological criteria include age, tumor grade, T stage and N stage status.
* *The reported molecular subtypes of PDAC were defined by a 62-gene signature, “PDAssigner”; Collisson EA, et al. Nat. Med. 2011; 17: 500-503.
* * *The six-gene metastasis signature includes FBJ murine osteosarcoma viral oncogene homolog B (FOSB), Kruppel-like factor 6 (KLF6), nuclear factor of kappa light polypeptide gene enhancer in B-cells inhibitor, zeta (NFKBIZ), ATPase H+/K+ exchanging, alpha polypeptide (ATP4A), germ cell associated 1 (GSG1), and sialic acid binding Ig-like lectin 11 (SIGLEC11); Stratford JK, et al. PLoS Med. 2010; 7(7): e1000307.

### Example 8

This example describes a six-gene prognostic model of PDAC based on the expression levels of ATP9A, ASPM, ACOX3, CDC45L, SLC40A1, and AGR2.

We investigated if we could use the expression levels of the six top-ranked genes in TABLE 2, including ATP9A, ASPM, ACOX3, CDC45L, SLC40A1, and AGR2 (Cox regression P value<0.005), to establish an effective prognostic model of PDAC. We calculated the risk score (Equation 1) based on the transcript levels of these genes in independent cohorts of patients with PDAC, including the UCSF cohort, the JHMI cohort, and the NW/NSF cohort. The patients were stratified into high-risk or low-risk group according to the risk score with the threshold determined by the maximal Youden's index.

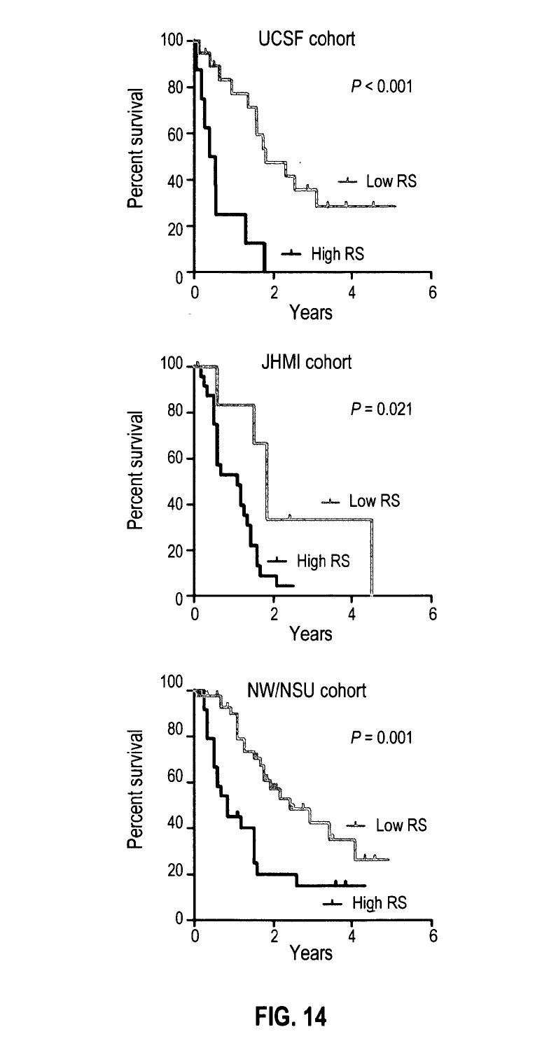

As shown in FIG. 14, based on the risk score, the staining intensities of ATP9A, ASPM, ACOX3, CDC45L, SLC40A1, and AGR2 could very effectively stratify risk of death by Kaplan-Meier analysis in each of the patient cohorts (log-rank test P=0.001-0.021).

As shown in TABLE 7, multivariate Cox regression analysis demonstrates that this six-gene model provides strong prognostic information to PDAC independent of clinical and pathological criteria in each of the three PDAC data sets.

TABLE 7
Multivariate Cox regression model predicting overall survival by the
six-gene model and clinico-pathological criteria in three independent
cohorts of patients with PDAC

| Variables | Hazard ratio | 95% CI | P-value |
|---|---|---|---|
| UCSF cohort | | | |
| Patient age | 1.255 | 0.767-2.052 | 0.370 |
| (per 10 years) | | | |
| Tumor grade (3 vs. <3) | 8.406 | 2.09-33.805 | 0.003* |
| T stage (3 vs. <3) | 1.925 | 0.487-7.606 | 0.350 |
| N stage (1 vs. 0) | 4.665 | 0.919-23.685 | 0.063 |
| Risk score by six-gene | 15.364 | 2.659-88.783 | 0.002* |
| model (high vs. low)** | | | |
| JHMI cohort | | | |
| Patient age | 0.883 | 0.644-1.211 | 0.441 |
| (per 10 years) | | | |
| Tumor grade (3 vs. <3) | 1.075 | 0.449-2.577 | 0.871 |
| T stage (3 vs. <3) | 0.867 | 0.312-2.406 | 0.783 |
| N stage (1 vs. 0) | 0.526 | 0.107-2.579 | 0.428 |
| Risk score by six-gene | 3.249 | 0.867-12.174 | 0.080 |
| model (high vs. low)** | | | |
| NW/NSU cohort | | | |
| Patient age | 1.482 | 1.025-2.142 | 0.036 |
| (per 10 years) | | | |
| Tumor grade (3 vs. <3) | 1.272 | 0.542-2.983 | 0.581 |
| T stage (3 vs. <3) | 0.969 | 0.285-3.291 | 0.959 |
| N stage (1 vs. 0) | 2.774 | 1.146-6.714 | 0.024 |
| Risk score by six-gene | 3.514 | 1.483-8.324 | 0.004* |
| model (high vs. low)** | | | |

CI, confidence interval.
*P < 0.05
**The threshold was determined by the maximal Youden's index.

TABLE 8 shows that, according to C-index values, the predictive accuracy of the six-gene model outperformed a combined clinical model and several previously reported prognostic gene signatures of PDAC in three independent data sets.

TABLE 8 shows that, according to C-index values, the predictive accuracy of the six-gene model outperformed a combined clinical model and several previously reported prognostic gene signatures of PDAC in three independent data sets.

TABLE 8
The prediction accuracy, as evaluated by C-index, of the six-gene
model and different prognosis prediction models in three independent
cohorts of patients with PDAC

| | C-index | 95% CI | P-value |
|---|---|---|---|
| UCSF cohort | | | |
| Clinico-pathological criteria* | 0.802 | 0.720-0.884 | |
| Risk score by six-gene model | 0.950 | 0.896-1.000 | 0.001 |
| Risk score by PDAssigner* * | 0.805 | 0.692-0.919 | 0.477 |
| Risk score by metastasis signature* * * | 0.573 | 0.402-0.744 | 0.993 |
| JHMI cohort | | | |
| Clinico-pathological criteria* | 0.574 | 0.491-0.656 | |
| Risk score by six-gene model | 0.833 | 0.663-1.000 | 0.002 |
| Risk score by PDAssigner* * | 0.586 | 0.448-0.724 | 0.431 |
| Risk score by metastasis signature* * * | 0.684 | 0.569-0.798 | 0.084 |
| NW/NSU cohort | | | |
| Clinico-pathological criteria* | 0.672 | 0.574-0.771 | |
| Risk score by six-gene model | 0.812 | 0.678-0.946 | 0.032 |
| Risk score by PDAssigner* * | 0.686 | 0.588-0.784 | 0.410 |
| Risk score by metastasis signature* * * | 0.640 | 0.538-0.743 | 0.678 |

C-index, concordance index; CI, confidence interval.
*Clinico-pathological criteria include age, tumor grade, T stage and N stage status.
* *The reported molecular subtypes of PDAC were defined by a 62-gene signature, “PDAssigner”; Collisson EA, et al. Nat. Med. 2011; 17: 500-503.
* * *The six-gene metastasis signature includes FBJ murine osteosarcoma viral oncogene homolog B (FOSB), Kruppel-like factor 6 (KLF6), nuclear factor of kappa light polypeptide gene enhancer in B-cells inhibitor, zeta (NFKBIZ), ATPase H+/K+ exchanging, alpha polypeptide (ATP4A), germ cell associated 1 (GSG1), and sialic acid binding Ig-like lectin 11 (SIGLEC11); Stratford JK, et al. PLoS Med. 2010; 7(7): e1000307.

### Example 9

This example describes a three-gene prognostic model of PDAC based on the expression levels of ASPM, ATP9A, and ACOX3.

We investigated if we could use the expression levels of the three top-ranked genes in TABLE 2, including ASPM, ATP9A, and ACOX3, to establish an effective prognostic model of PDAC. We calculated the risk score (Equation 1) based on the transcript levels of ASPM, ATP9A, and ACOX3 in independent cohorts of patients with PDAC, including the UCSF cohort, the JHMI cohort, and the NW/NSF cohort. The patients were stratified into high-risk or low-risk group according to the risk score with the threshold determined by the maximal Youden's index.

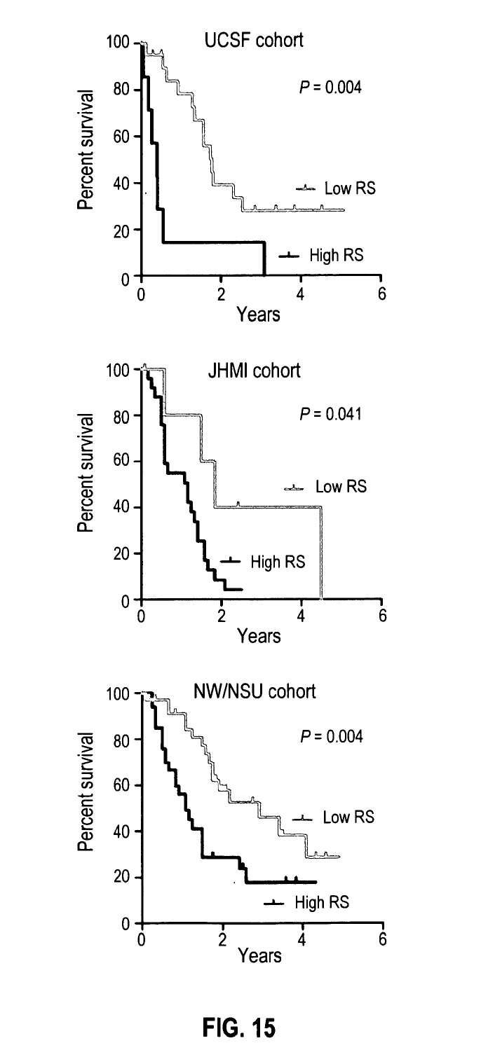

As shown in FIG. 15, based on the risk score, the transcript levels of ASPM, ATP9A, and ACOX3 could effectively stratify risk of death by Kaplan-Meier analysis in each of the patient cohorts (log-rank test P=0.004-0.041).

As shown in TABLE 9, multivariate Cox regression analysis demonstrates that this three-gene model provides strong prognostic information to PDAC independent of clinical and pathological criteria in each of the three data sets.

TABLE 9
Multivariate Cox regression model predicting overall survival by the
three-gene model and clinico-pathological criteria in independent cohorts
of patients with PDAC

| Variables | Hazard ratio | 95% CI | P-value |
|---|---|---|---|
| UCSF cohort | | | |
| Patient age | 1.351 | 0.829-2.201 | 0.230 |
| (per 10 years) | | | |
| Tumor grade (3 vs. <3) | 3.168 | 0.777-12.914 | 0.110 |
| T stage (3 vs. <3) | 1.390 | 0.366-5.279 | 0.630 |
| N stage (1 vs. 0) | 11.728 | 1.61-85.411 | 0.015* |
| Risk score by three-gene | 8.449 | 1.272-56.113 | 0.027* |
| model (high vs. low)** | | | |
| JHMI cohort | | | |
| Patient age | | | |
| (per 10 years) | 0.852 | 0.623-1.166 | 0.316 |
| Tumor grade (3 vs. <3) | 1.178 | 0.497-2.79 | 0.710 |
| T stage (3 vs. <3) | 0.753 | 0.28-2.021 | 0.573 |
| N stage (1 vs. 0) | 1.050 | 0.22-5.018 | 0.951 |
| Risk score by three-gene | 2.852 | 0.776-10.479 | 0.115 |
| model (high vs. low)** | | | |
| NW/NSU cohort | | | |
| Patient age | 1.489 | 1.038-2.135 | 0.031* |
| (per 10 years) | | | |
| Tumor grade (3 vs. <3) | 1.752 | 0.778-3.945 | 0.175 |
| T stage (3 vs. <3) | 0.764 | 0.253-2.308 | 0.633 |
| N stage (1 vs. 0) | 2.347 | 1.03-5.345 | 0.042 |
| Risk score by three-gene | 3.777 | 1.665-8.57 | 0.001* |
| model (high vs. low)** | | | |

CI, confidence interval.
*P < 0.05
**The threshold was determined by the maximal Youden's index.

TABLE 10 shows that, according to C-index values, the predictive accuracy of the three-gene model outperformed a combined clinical model including age, tumor grade, and clinical stage in each of the three PDAC data sets.

TABLE 10
The prediction accuracy, as evaluated by C-index, of the three-
gene model and clinico-pathological criteria in three independent cohorts
of patients with PDAC

| | C-index | 95% CI | P-value |
|---|---|---|---|
| UCSF cohort | | | |
| Clinico-pathological criteria* | 0.802 | 0.720-0.884 | |
| Risk score by three-gene model | 0.899 | 0.727-1.000 | 0.120 |
| JHMI cohort | | | |
| Clinico-pathological criteria* | 0.574 | 0.491-0.656 | |
| Risk score by three-gene model | 0.805 | 0.599-1.000 | 0.017 |
| NW/NSU cohort | | | |
| Clinico-pathological criteria* | 0.672 | 0.574-0.771 | |
| Risk score by three-gene model | 0.833 | 0.716-0.950 | 0.011 |

C-index, concordance index; CI, confidence interval.
*Clinico-pathological criteria include age, tumor grade, T stage and N stage status.

### Example 10

This example describes the calculation of predicted recurrence rate and expected recurrence-free survival for patients with pancreatic cancer in based on the 28-gene prognostic model shown in Example 2.

As described in Example 3, one can measure the risk of post-operative recurrence of a given patient with pancreatic cancer by calculating the Risk Score (Risk score=Σᵢ₌₃^{k}bᵢxᵢ (Equation 1) based on a selected gene set. For a patient whose risk score is known, the hazard rate of recurrence at time t of said patient can be estimated by Cox regression, and the hazard rate can be expressed as h(t)=h₀(t)exp(bx), where x is the value of risk score, b is the regression coefficient, and h₀(t) is the baseline hazard function. The predicted survival rate at time t can be estimated according to:

S(t)=S₀(t)^{exp(bx)} (Equation 2)

where S₀(t)=exp[−∫₀^{t}h₀(u)du] is the baseline survival function. The calculation can be carried out by commercial software such as the SPSS software=S0(t)exp(bx) (Equation 2 as setting St=0.5

For example, the risk score of a given patient in the UCSF cohort can be calculated based on the transcript abundance levels of the 28 gene markers of said subject as follows:

x=−4.792+(−5.608 ATP9A+4.114 ASPM−5.821 ACOX3+12.814 CDC45L−2.026 SLC40A1−1.700 AGR2+9.861 ATP11C+4.137 FAM72A//B//C//D−5.444 PLA2G10−3.591 MATN2+5.767 APITD1+12.789 KIF11−2.828 HPGD+3.087 HMMR−2.483 ELF3+4.903 PTTG1+10.213 UPP1+7.367 CCNB2−5.104 CREG1−3.033 ARSD+6.097 CENPN+3.243 SMC4+4.948 DLGAP5−3.524 PIK3AP1−7.569 TLR3+2.415 TWIST1+5.639 GCLM−3.480 CTSS)/28 (Equation 3)

The estimated Cox regression is h(t)=h₀(t)exp(1.053x). The survival function can be represented by S(t)=S₀(t)^{exp(1.053x)}(:

S(t)=S₀(t)^{exp(1.053x)} (Equation 4)

The values of estimated S₀(t) are shown in TABLE 11.

TABLE 11
Baseline survival rates of patients in the UCSF cohort estimated
according to the Cox regression based on the risk score calculated
using the 28-gene model.

| t | S₀(t) |
|---|---|
| [0, 0.055) | 1.000 |
| [0.055, 0.123) | 0.994 |
| [0.123, 0.178) | 0.989 |
| [0.178, 0.263) | 0.982 |
| [0.263, 0.394) | 0.971 |
| [0.394, 0.402) | 0.940 |
| [0.402, 0.545) | 0.871 |
| [0.545, 0.553) | 0.795 |
| [0.553, 0.643) | 0.720 |
| [0.643, 0.936) | 0.647 |
| [0.936, 1.292) | 0.576 |
| [1.292, 1.347) | 0.504 |
| [1.347, 1.563) | 0.433 |
| [1.563, 1.574) | 0.367 |
| [1.574, 1.73) | 0.304 |
| [1.73, 1.774) | 0.241 |
| [1.774, 1.796) | 0.182 |
| [1.796, 2.311) | 0.127 |
| [2.311, 2.538) | 0.080 |
| [2.538, 3.086) | 0.045 |
| [3.086, ∞) | 0.015 |

TABLE 12 shows the observed and predicted survival in four PDAC patients selected from the UCSF cohort.

TABLE 12
Overall survival and one-year survival rate of selected patients in
the UCSF cohort as predicted by the 12-gene model

| Transcript abundance level* | Patient 1 | Patient 2 | Patient 3 | Patient 4 |
|---|---|---|---|---|
| ATP9A | 8.583 | 8.311 | 7.825 | 6.904 |
| ASPM | 4.000 | 4.966 | 4.711 | 5.717 |
| ACOX3 | 7.302 | 6.946 | 6.583 | 5.002 |
| CDC45L | 4.304 | 4.519 | 4.621 | 4.459 |
| SLC40A1 | 10.886 | 11.181 | 10.647 | 6.457 |
| AGR2 | 11.602 | 11.180 | 10.873 | 7.109 |
| ATP11C | 6.190 | 6.758 | 6.806 | 7.369 |
| FAM72A///FAM72B/// | 3.197 | 3.910 | 3.599 | 4.903 |
| FAM72C///FAM72D | | | | |
| PLA2G10 | 7.807 | 6.857 | 7.130 | 6.494 |
| MATN2 | 5.963 | 4.013 | 4.217 | 3.843 |
| APITD1 | 5.127 | 5.925 | 6.508 | 6.348 |
| KIF11 | 2.902 | 2.927 | 2.801 | 3.332 |
| HPGD | 5.049 | 4.603 | 5.232 | 4.188 |
| HMMR | 3.675 | 3.997 | 4.167 | 5.931 |
| ELF3 | 6.798 | 7.474 | 7.069 | 5.630 |
| PTTG1 | 6.371 | 6.893 | 6.789 | 7.881 |
| UPP1 | 4.850 | 4.626 | 5.275 | 5.194 |
| CCNB2 | 4.609 | 4.656 | 4.777 | 5.557 |
| CREG1 | 5.174 | 5.276 | 4.415 | 3.814 |
| ARSD | 7.199 | 8.360 | 6.900 | 7.000 |
| CENPN | 4.119 | 3.997 | 3.910 | 4.185 |
| SMC4 | 4.523 | 3.505 | 4.749 | 5.904 |
| DLGAP5 | 3.373 | 3.307 | 3.737 | 4.317 |
| PIK3AP1 | 7.112 | 6.212 | 5.903 | 4.640 |
| TLR3 | 5.069 | 5.003 | 4.935 | 4.972 |
| TWIST1 | 6.476 | 5.683 | 7.186 | 9.216 |
| GCLM | 3.956 | 4.177 | 4.207 | 5.132 |
| CTSS | 8.155 | 6.404 | 6.732 | 5.744 |
| Risk score by the 28-gene | −2.453 | −1.096 | 0.007 | 3.720 |
| model | | | | |
| Observed survival (years) | 3.841 | 2.861 | 1.292 | 0.178 |
| Predicted survival (years) | >3.086 | 2.311 | 1.347 | 0.178 |
| Death before one year | No | No | No | Yes |
| Predicted one-year survival | 95.9% | 84.0% | 57.4% | <0.1% |
| rate | | | | |

*Transcript abundance levels measured by Human GeneChip U133Plus2.0® arrays (Affymetrix) and expressed as probe hybridization intensities. The data and the associated clinical information were downloaded from Gene Expression Omnibus (GSE17891) (Collisson et al., 2011).

### Example 11

This example describes the calculation of predicted recurrence rate and expected recurrence-free survival for patients with pancreatic cancer based on the six-gene prognostic model shown in Example 8.

The same principle in Example 10 can be used to apply the six-gene model as shown in Example 8 to predict the recurrence rate and expected recurrence-free survival in patients in the UCSF cohort. According to the Risk Score Risk score=Σᵢ₌₃^{k}bᵢxᵢ (Equation 1), one can calculate the risk score of a given patient in the UCSF cohort based on the staining intensities, as represented by the H-scores, of ATP9A, ASPM, ACOX3, CDC45L, SLC40A1, and AGR2 in the tumor of said patient using the following equation:

x=7.235+(−5.608 ATP9A+4.114 ASPM−5.821 ACOX3+12.814 CDC45L−2.026 SLC40A1−1.7 AGR2)/6) (Equation 5)

The estimated Cox regression is h(t)=h₀(t)exp(0.580x). The survival function can be represented by:

S(t)=1−S₀(t)^{exp(0.580x)} (Equation 6)

TABLE 13 shows the values of the estimated S₀(t):

TABLE 13
Baseline survival rates of patients in the UCSF cohort estimated
according to the Cox regression based on the risk score calculated
using the six-gene model.

| t | S₀(t) |
|---|---|
| [0, 0.055) | 1.000 |
| [0.055, 0.123) | 0.990 |
| [0.123, 0.178) | 0.979 |
| [0.178, 0.263) | 0.968 |
| [0.263, 0.394) | 0.949 |
| [0.394, 0.402) | 0.910 |
| [0.402, 0.545) | 0.846 |
| [0.545, 0.553) | 0.780 |
| [0.553, 0.643) | 0.713 |
| [0.643, 0.936) | 0.646 |
| [0.936, 1.292) | 0.582 |
| [1.292, 1.347) | 0.519 |
| [1.347, 1.563) | 0.458 |
| [1.563, 1.574) | 0.400 |
| [1.574, 1.73) | 0.345 |
| [1.73, 1.774) | 0.292 |
| [1.774, 1.796) | 0.241 |
| [1.796, 2.311) | 0.192 |
| [2.311, 2.538) | 0.148 |
| [2.538, 3.086) | 0.108 |
| [3.086, ∞) | 0.066 |

TABLE 14 shows the observed and predicted survival of four PDAC patients selected from the UCSF cohort.

TABLE 14
Overall survival and one-year survival rate of selected patients in
the UCSF cohort as predicted by the six-gene model

| Transcript abundance level* | Patient 1 | Patient 2 | Patient 3 | Patient 4 |
|---|---|---|---|---|
| ATP9A | 7.943 | 7.739 | 7.825 | 6.904 |
| ASPM | 4.220 | 4.091 | 4.711 | 5.717 |
| ACOX3 | 6.185 | 6.244 | 6.583 | 5.002 |
| CDC45L | 4.224 | 4.357 | 4.621 | 4.459 |
| SLC40A1 | 10.155 | 10.754 | 10.647 | 6.457 |
| AGR2 | 10.283 | 11.284 | 10.873 | 7.109 |
| Risk score by the six-gene | −2.900 | −0.774 | −0.042 | 5.177 |
| model | | | | |
| Observed survival (years) | 3.841 | 1.730 | 1.292 | 0.178 |
| Predicted survival (years) | >3.086 | 1.730 | 1.347 | 0.263 |
| Death after one year | No | No | No | Yes |
| Predicted one-year survival | 90.4% | 70.8% | 59.0% | <0.1% |
| rate | | | | |

*Transcript abundance levels measured by Human GeneChip U133Plus2.0® arrays (Affymetrix) and expressed as probe hybridization intensities. The data and the associated clinical information were downloaded from Gene Expression Omnibus (GSE17891) (Collisson et al., 2011).

### Example 12

This example describes the expression and the prognostic value of ASPM in human breast cancer.

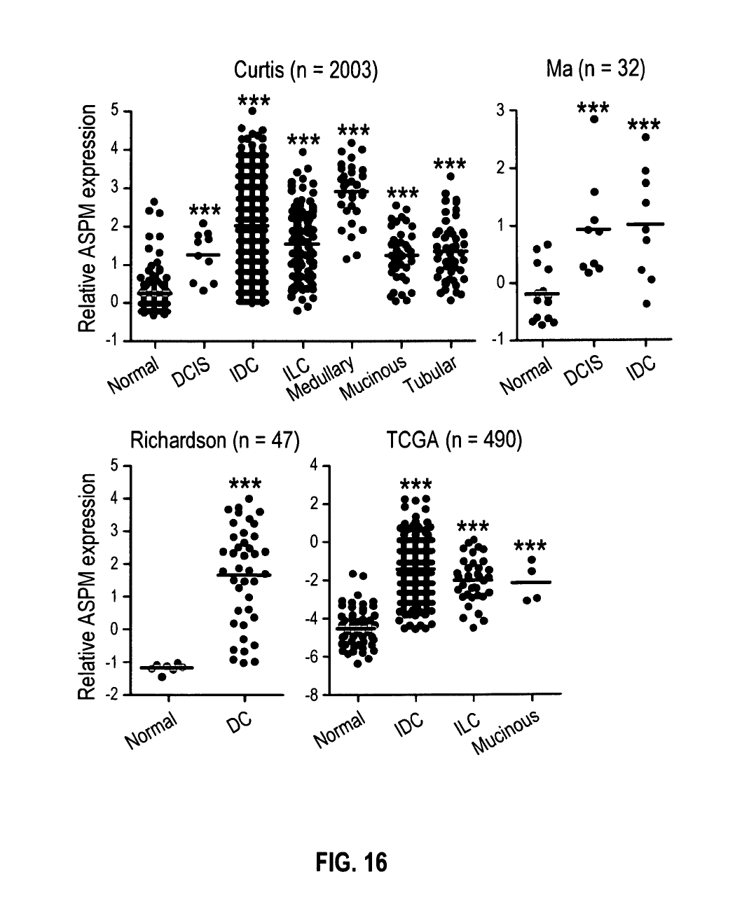

As shown in FIG. 16, by interrogating published tumor transcriptome data sets from Oncomine (www.oncomine.org), we uncovered that the transcript level of ASPM significantly increased in different pathologic subtypes of human breast cancers relative to normal breast tissues in several large cohorts of patients with breast cancer. To investigate if the up-regulated ASPM expression correlates with the clinical prognosis of patients with breast cancer, we interrogated ASPM expression from a published breast cancer transcriptome data set derived from several large cohorts (n=2416 in total) of breast cancer patients (Curtis et al., 2012; Pawitan et al., 2005; Wang et al., 2005). We correlated ASPM expression levels with clinical outcome (overall survival or relapse-free survival) in these patients.

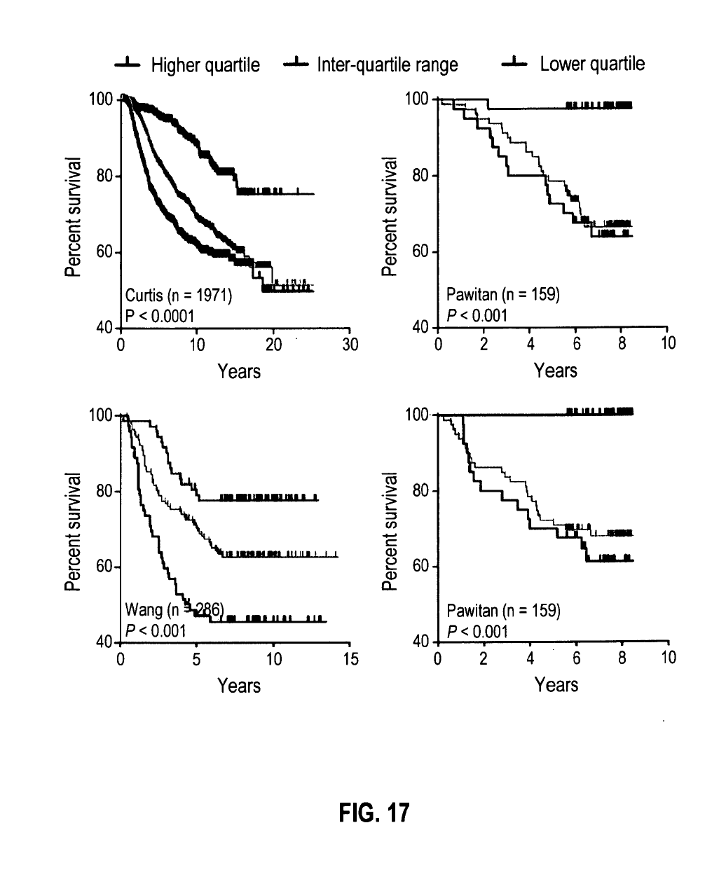

As shown in FIG. 17, when the patients in the Curtis cohort (Curtis et al., 2012) were grouped according to ASPM expression quartiles, the patients with their tumors expressing higher expression levels of ASPM had significantly shorter overall survival than those with tumors expressing intermediate or lower levels of ASPM (log-rank test P<0.0001).

Similarly, when the patients in the Pawitan cohort (Pawitan et al., 2005) were grouped according to ASPM expression quartiles, the patients with their tumors expressing higher expression levels of ASPM had significantly shorter overall or relapse-free survival than those with tumors expressing intermediate or lower levels of ASPM (log-rank test P<0.001). The same reverse correlation between ASPM expression and survival was observed in another cohort of patients with breast cancer (Wang et al., 2005) with a log-rank P value less than 0.001.

As shown in TABLE 15, multivariate Cox regression analysis demonstrates that, compared to pathological tumor grading and the molecular subtypes of breast cancer, the transcript level of ASPM provides the strongest and independent prognostic information with a hazard ratio for post-operative disease relapse reaching 4.428 (P=0.002).

TABLE 15
Multivariate Cox regression model predicting relapse-free survival
by the expression level of ASPM and tumor grade and molecular subtypes
of breast cancer

| | Hazard | | |
|---|---|---|---|
| | ratio | 95% CI | P-value |
| ASPM (high vs. low)* | 4.428 | 1.710-11.467 | 0.002 |
| Tumor grade | | | |
| Grade 2 vs. grade 1 | 2.804 | 0.619-12.705 | 0.181 |
| Grade 3 vs. grade 1 | 2.612 | 0.550-12.410 | 0.227 |
| Molecular subtype | | | |
| Normal-like & luminal B vs. | 1.667 | 0.605-4.592 | 0.323 |
| luminal A | | | |
| Basal & ERBB2+ vs. luminal A | 1.263 | 0.444-3.591 | 0.661 |

The analysis included the 159 breast cancer patients in the Stockholm cohort (Pawitan et al., 2005).
C.I., confidence interval.
*The cut-off-value for ASPM was selected by using the maximal Youden's index.

As shown in TABLE 16, multivariate Cox regression analysis demonstrates that the transcript level of ASPM provides the strongest prognostic information to breast cancer independent of pathological criteria and the molecular subtypes of breast cancer (hazard ratio=3.669; P=0.011).

TABLE 16
Multivariate Cox regression model predicting overall survival by
the expression level of ASPM and tumor grade and molecular subtypes
of breast cancer

| | Hazard | | |
|---|---|---|---|
| | ratio | 95% CI | P-value |
| ASPM (high vs. low)* | 3.669 | 1.347-9.991 | 0.011 |
| Tumor grade | | | |
| Grade 2 vs. grade 1 | 2.164 | 0.469-9.984 | 0.322 |
| Grade 3 vs. grade 1 | 1.828 | 0.374-8.924 | 0.456 |
| Molecular subtype | | | |
| Normal-like & luminal B vs. | 0.878 | 0.319-2.414 | 0.801 |
| luminal A | | | |
| Basal & ERBB2+ vs. luminal A | 1.211 | 0.450-3.258 | 0.705 |

The analysis included the 159 breast cancer patients in the Stockholm cohort (Pawitan et al., 2005).
C.I., confidence interval.
*The cut-off-value for ASPM was selected by using the maximal Youden's index.

### Example 13

This example describes the role of ASPM in breast cancer proliferation, migration, Wnt activity and stemness and the therapeutic effect of ASPM inhibition.

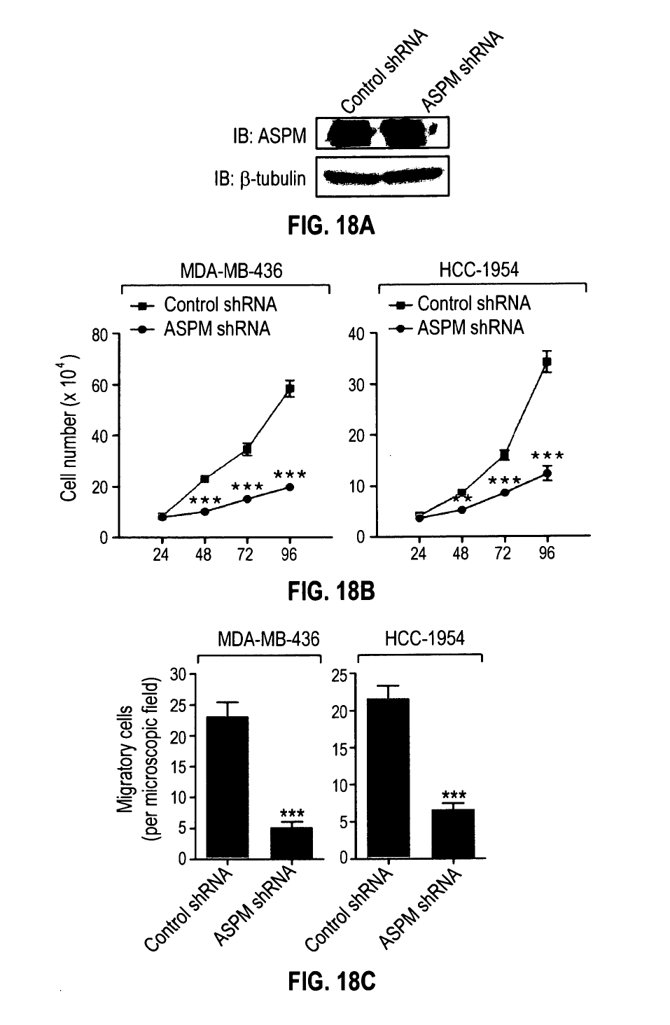

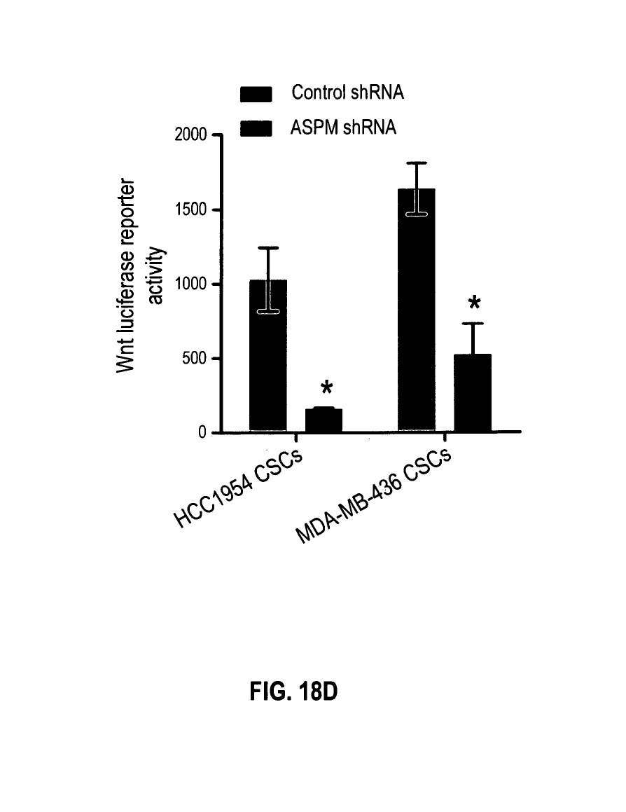

Given that ASPM is a strong and robust poorly prognostic factor in breast cancer as shown in Example 12, we assess if ASPM also plays a role in the malignant behaviors of breast cancer cells and their Wnt activity. To this end, we stably down-regulated the expression of ASPM in breast cancer cells by using lentivirus-mediated RNAi as described in Example 4. FIG. 18A shows the level of ASPM knockdown as verified by immunoblot analysis. FIG. 18B shows that, similar to the findings in PDAC cells, knockdown of endogenous ASPM expression in metastatic breast cancer MDA-MB-436 or primary tumor-derived HCC-1954 cells could respectively attenuate cellular proliferation.

To access the effect of ASPM silencing on the migratory capacity of breast cancer cells, we conducted modified Boyden chamber assay as described in Example 4. As shown in FIG. 18C, silencing of ASPM expression significantly attenuated cellular migration of MDA-MB-436 and HCC-1954 cells in response to breast carcinoma-associated fibroblasts.

To further assess if ASPM regulates Wnt pathway activity in breast cancer cells, we expressed a Wnt reporter construct in both MDA-MB-436 and HCC-1954 cells as described in Example 4. FIG. 18D shows that, when the Wnt signaling was activated in MDA-MB-436 or HCC-1954 cells by Wnt-3a, cells with silenced ASPM expression exhibited dramatically blunted Wnt-mediated luciferase reporter activation. This result confirmed that ASPM is also functionally important for the Wnt signaling pathway activity in breast cancer cells.

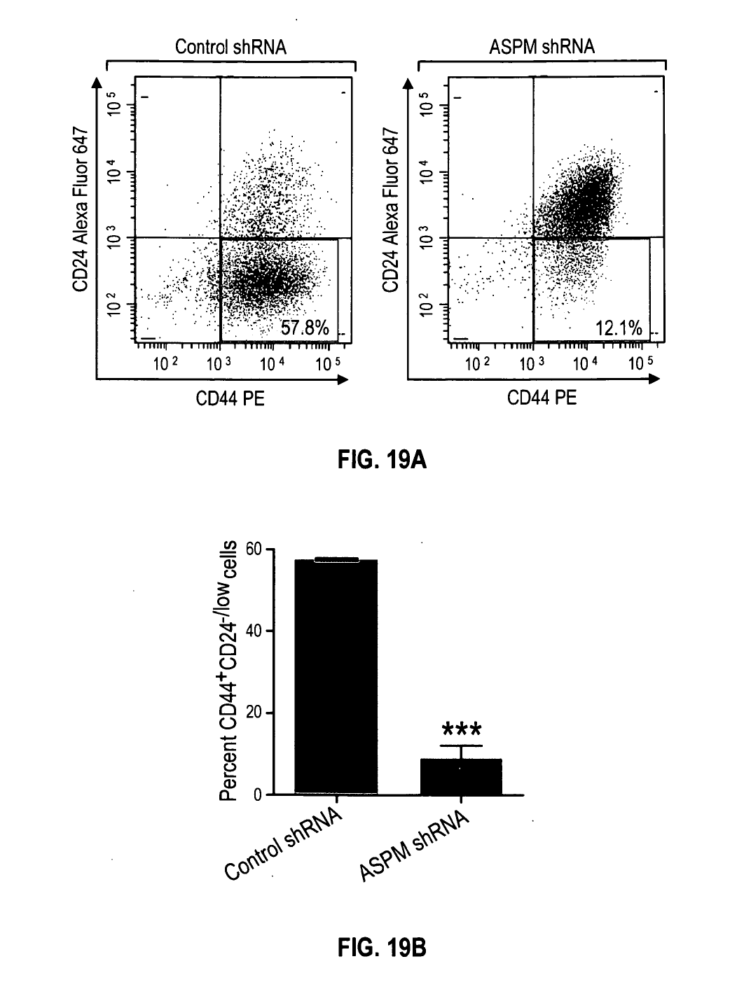

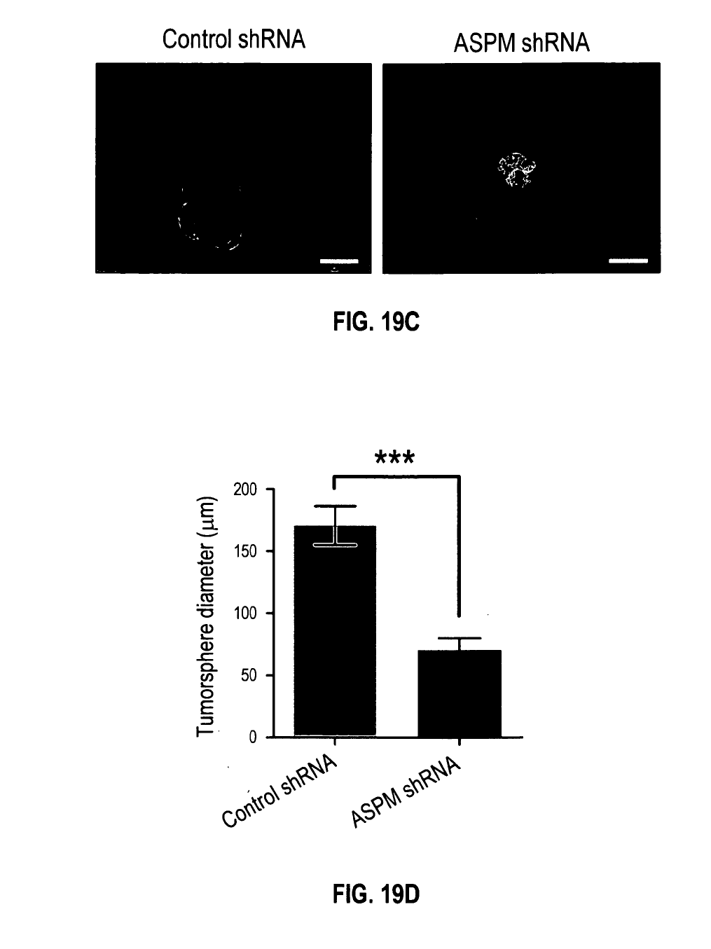

The findings that ASPM also supports the proliferation, migration and Wnt activity in breast cancer cells promoted us to speculate that it may also play a critical in the regulation of stem-like cells in breast cancer. To this end, we measured the proportion of cells with a CD44⁺CD24^{-/low} phenotype, which contains the enriched cancer stem-like cells in breast cancer (Al-Hajj et al., 2003), in control or ASPM shRNA-transduced breast cancer MDA-MB-436 cells. Cells were dissociated, antibody-labeled and resuspended in HBSS/2% FBS containing DAPI as previously described (Li et al., 2007). The antibodies used included PE anti-CD44, and Alexa Fluor 647 anti-CD24 (BD Biosciences). Flow cytometry was done using a FACSCanto II flow cytometer (BD Biosciences). As shown in FIG. 19A and FIG. 19B, knockdown of ASPM led to a substantial reduction (48.5% on average) of the CD44⁺CD24^{-/low} tumor cell population.

To explore the functional relevance of the above finding, we preformed tumorsphere assay on flow-sorted CD44⁺CD24^{-/low} MDA-MB-436 cells. CD44^{hi} CD24^{low} and CD44^{hi}CD24^{hi} cells were sorted by FACS (BD FACSAria™ III cell sorter; BD Biosciences) as previously described (Ginestier et al., 2007). Briefly, tumorspheres were maintained on ultralow adherent plates (Corning Inc., Lowell, Mass., USA) in MammoCult media according to the manufacturer's instructions (StemCell Technologies). Equal numbers of live cells were plated in ultralow attachment plates to generate the primary spheres. After 7 days, the mammospheres's sizes were measured and pictures were taken. FIG. 19C and FIG. 19D clearly shows that knockdown of ASPM substantially reduced the growth and the size of the tumorspheres. Together, these data suggests that ASPM is an important regulator of breast cancer stemness.

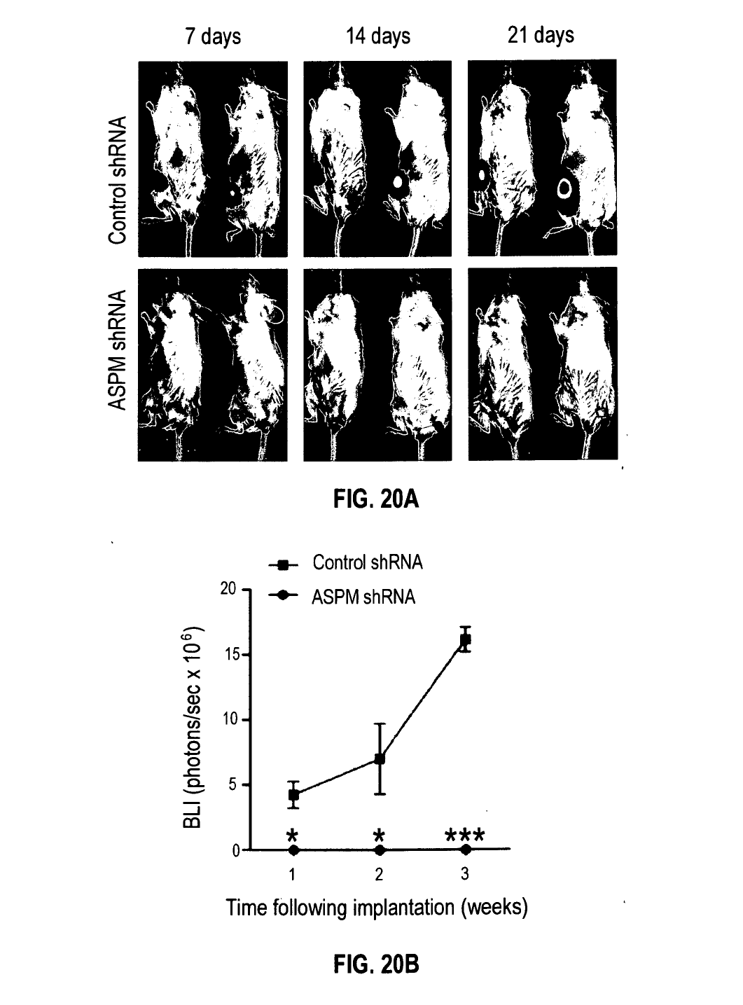

The role of ASPM in breast cancer growth, migration and stemness raised the possibility that it may contribute to breast cancer progression in vivo. To address this possibility, we stably expressed a firefly luciferase reporter in control or ASPM shRNA-transduced breast cancer MDA-MB-436 cells and ortho-topically implanted them into the mammary fat pads of NOD-SCID mice. As shown in FIG. 20, silencing of ASPM completely crippled the ability of breast cancer cells to initiate tumor growth in vivo while animals harboring control shRNA tumors exhibited significant growth over a period of 4 weeks following transplantation.

### Example 14

This example describes the role of ASPM in prostate cancer proliferation and migration and the therapeutic effect of ASPM inhibition.

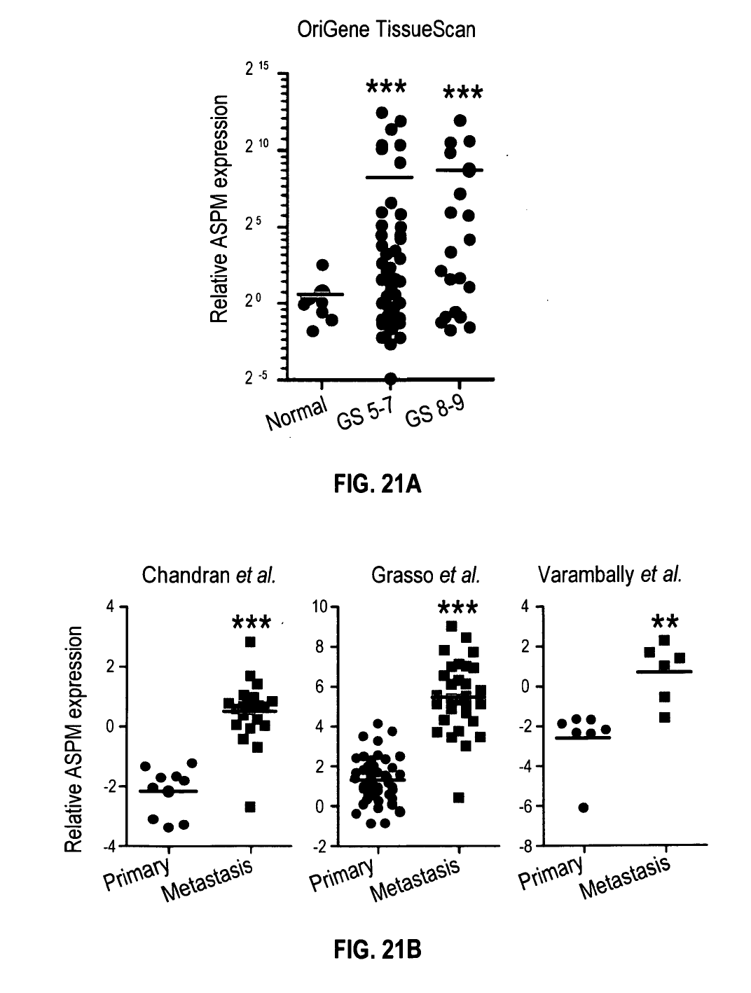

Given that ASPM is a critical regulator of cell proliferation, migration, stemness and tumor progression in PDAC and breast cancer, we assess if ASPM also plays a role in the malignant behaviors of prostate cancer cells, another type of gland-derived cancers. By measuring the transcript level of ASPM in a series of normal or malignant prostate tissues, we uncovered that ASPM is significantly up-regulated in prostate cancer compared with normal tissues FIG. 21A. Moreover, by interrogating published tumor transcriptome data sets from Oncomine (www.oncomine.org), we uncovered that the transcript level of ASPM significantly increased in metastatic prostate cancer compared with primary tumor. These clinical correlative analyses support the possibility that ASPM plays an important role in prostate cancer initiation and progression.

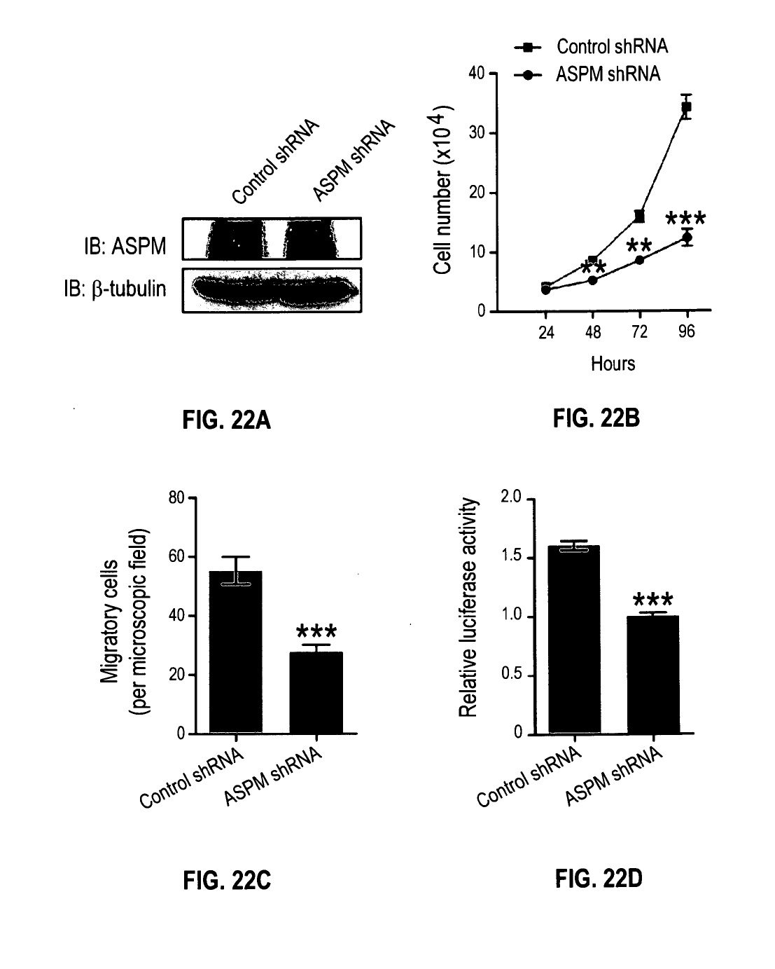

To address the functional importance of ASPM in prostate cancer, we stably down-regulated the expression of ASPM in prostate cancer PC-3 cells by using lentivirus-mediated RNAi as described in Example 4. FIG. 22A shows the level of ASPM knockdown as verified by immunoblot analysis. FIG. 22B shows that, similar to the findings in PDAC cells, knockdown of endogenous ASPM expression in PC-3 cells could respectively attenuate cellular proliferation.

To access the effect of ASPM silencing on the migratory capacity of prostate cancer cells, we conducted modified Boyden chamber assay as described in Example 4. As shown in FIG. 22C, silencing of ASPM expression significantly attenuated cellular migration of PC-3 cells in response to prostate stromal WPMY-1 cells (American Type Culture Collection).

To assess if ASPM regulates Wnt pathway activity, we expressed a Wnt reporter construct in prostate cancer PC-3 cells. Cells were transduced with Cignal Lenti TCF/LEF Reporter (Qiagen) according to the manufacturer's protocol. Following stimulation of the cells with recombinant human Wnt-3a (250 ng/mL for 16 hours; R&D Systems, Minneapolis, Minn.) or vehicle the reporter activity was measured by using the ONE-Glo™ Luciferase Assay System (Promega, Madison, Wis.). As shown in FIG. 22D, when the Wnt signaling was activated in PC-3 cells by the canonical Wnt ligand Wnt-3a, cells depleted with ASPM exhibited dramatically blunted Wnt-mediated luciferase reporter activation. This result confirmed that ASPM is functionally important for the Wnt signaling pathway activity in prostate cancer cells.

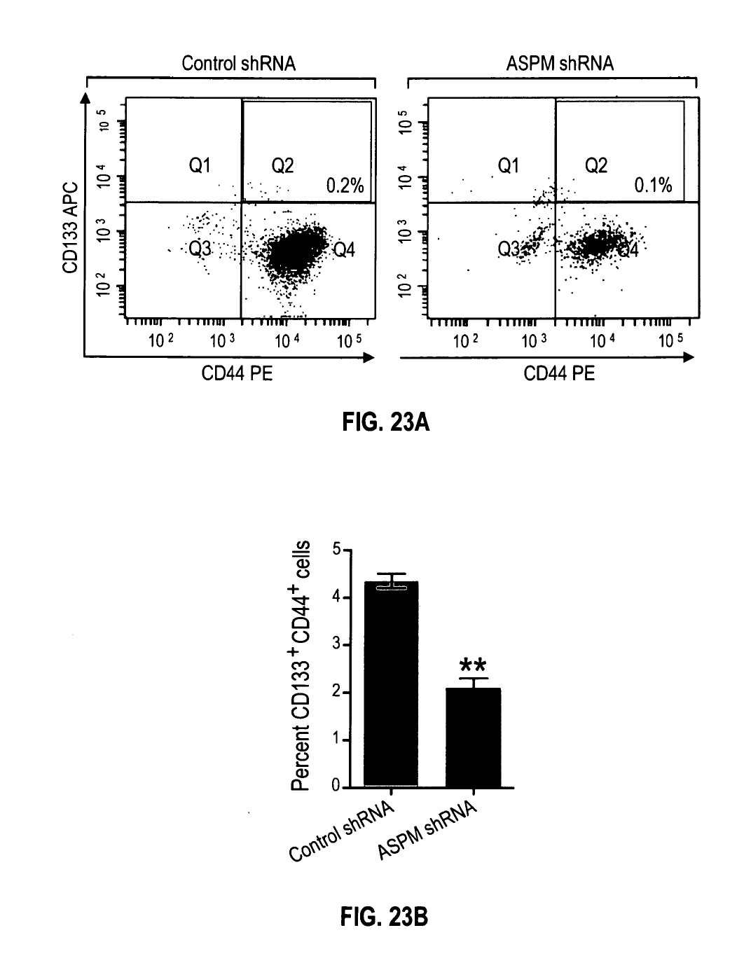

To further assess if ASPM also play a critical in the regulation of stem-like cells in prostate cancer, we measured the proportion of cells with a CD133⁺CD44⁺ phenotype, which contains the enriched cancer stem-like cells in breast cancer (Dubrovska et al., 2009), in control shRNA or ASPM shRNA-transduced prostate cancer PC-3 cells. Cells were dissociated, antibody-labeled and resuspended in HBSS/2% FBS containing DAPI as previously described (Li et al., 2007). The antibodies used included APC-anti-CD133, and PE-anti-CD44 (BD Biosciences). Flow cytometry was done using a FACSCanto II flow cytometer (BD Biosciences). As shown in FIG. 23, knockdown of ASPM led to a substantial reduction (51.7% on average) of the CD133⁺CD44⁺ tumor cell population, indicating that ASPM indeed contributes to prostate cancer stemness.

### REFERENCES

Adsay, N. V., Basturk, O., Bonnett, M., Kilinc, N., Andea, A. A., Feng, J., Che, M., Aulicino, M. R., Levi, E., and Cheng, J. D. (2005). A proposal for a new and more practical grading scheme for pancreatic ductal adenocarcinoma. Am J Surg Pathol 29, 724-733.

Al-Hajj, M., Wicha, M. S., Benito-Hernandez, A., Morrison, S. J., and Clarke, M. F. (2003). Prospective identification of tumorigenic breast cancer cells. Proc Natl Acad Sci USA 100, 3983-3988.

Arensman, M. D., Kovochich, A. N., Kulikauskas, R. M., Lay, A. R., Yang, P. T., Li, X., Donahue, T., Major, M. B., Moon, R. T., Chien, A. J., et al. (2013). WNT7B mediates autocrine Wnt/beta-catenin signaling and anchorage-independent growth in pancreatic adenocarcinoma. Oncogene.

Bello, D., Webber, M. M., Kleinman, H. K., Wartinger, D. D., and Rhim, J. S. (1997). Androgen responsive adult human prostatic epithelial cell lines immortalized by human papillomavirus 18. Carcinogenesis 18, 1215-1223.

Bikeye, S. N., Colin, C., Marie, Y., Vampouille, R., Ravassard, P., Rousseau, A., Boisselier, B., ldbaih, A., Calvo, C. F., Leuraud, P., et al. (2010). ASPM-associated stem cell proliferation is involved in malignant progression of gliomas and constitutes an attractive therapeutic target. Cancer Cell Int 10, 1.

Bond, J., Scott, S., Hampshire, D. J., Springell, K., Corry, P., Abramowicz, M. J., Mochida, G. H., Hennekam, R. C., Maher, E. R., Fryns, J. P., et al. (2003). Protein-truncating mutations in ASPM cause variable reduction in brain size. Am J Hum Genet 73, 1170-1177.

Bruning-Richardson, A., Bond, J., Alsiary, R., Richardson, J., Cairns, D. A., McCormack, L., Hutson, R., Burns, P., Wilkinson, N., Hall, G. D., et al. (2011). ASPM and microcephalin expression in epithelial ovarian cancer correlates with tumour grade and survival. British journal of cancer 104, 1602-1610.

Buchman, J. J., Durak, O., and Tsai, L. H. (2011). ASPM regulates Wnt signaling pathway activity in the developing brain. Genes Dev 25, 1909-1914.

Chandran, U. R., Ma, C., Dhir, R., Bisceglia, M., Lyons-Weiler, M., Liang, W., Michalopoulos, G., Becich, M., and Monzon, F. A. (2007). Gene expression profiles of prostate cancer reveal involvement of multiple molecular pathways in the metastatic process. BMC cancer 7, 64.

Chang, H. Y., Sneddon, J. B., Alizadeh, A. A., Sood, R., West, R. B., Montgomery, K., Chi, J. T., van de Rijn, M., Botstein, D., and Brown, P. O. (2004). Gene expression signature of fibroblast serum response predicts human cancer progression: similarities between tumors and wounds. PLoS Biol 2, E7.

Collisson, E. A., Sadanandam, A., Olson, P., Gibb, W. J., Truitt, M., Gu, S., Cooc, J., Weinkle, J., Kim, G. E., Jakkula, L., et al. (2011). Subtypes of pancreatic ductal adenocarcinoma and their differing responses to therapy. Nat Med 17, 500-503.

Curtis, C., Shah, S. P., Chin, S. F., Turashvili, G., Rueda, O. M., Dunning, M. J., Speed, D., Lynch, A. G., Samarajiwa, S., Yuan, Y., et al. (2012). The genomic and transcriptomic architecture of 2,000 breast tumours reveals novel subgroups. Nature 486, 346-352.

Debnath, J., and Brugge, J. S. (2005). Modelling glandular epithelial cancers in three-dimensional cultures. Nat Rev Cancer 5, 675-688.

Fournier, M. V., Martin, K. J., Kenny, P. A., Xhaja, K., Bosch, I., Yaswen, P., and Bissell, M. J. (2006). Gene expression signature in organized and growth-arrested mammary acini predicts good outcome in breast cancer. Cancer Res 66, 7095-7102.

Ginestier, C., Hur, M. H., Charafe-Jauffret, E., Monville, F., Dutcher, J., Brown, M., Jacquemier, J., Viens, P., Kleer, C. G., Liu, S., et al. (2007). ALDH1 is a marker of normal and malignant human mammary stem cells and a predictor of poor clinical outcome. Cell Stem Cell 1, 555-567.

Gleason, D. F. (1992). Histologic grading of prostate cancer: a perspective. Human pathology 23, 273-279.

Glinsky, G. V., Glinskii, A. B., Stephenson, A. J., Hoffman, R. M., and Gerald, W. L. (2004). Gene expression profiling predicts clinical outcome of prostate cancer. J Clin Invest 113, 913-923.

Gold, P., and Freedman, S. O. (1965). Demonstration of Tumor-Specific Antigens in Human Colonic Carcinomata by Immunological Tolerance and Absorption Techniques. J Exp Med 121, 439-462.

Grasso, C. S., Wu, Y. M., Robinson, D. R., Cao, X., Dhanasekaran, S. M., Khan, A. P., Quist, M. J., Jing, X., Lonigro, R. J., Brenner, J. C., et al. (2012). The mutational landscape of lethal castration-resistant prostate cancer. Nature 487, 239-243.

Grutzmann, R., Pilarsky, C., Ammerpohl, O., Luttges, J., Bohme, A., Sipos, B., Foerder, M., Alldinger, I., Jahnke, B., Schackert, H. K., et al. (2004). Gene expression profiling of microdissected pancreatic ductal carcinomas using high-density DNA microarrays. Neoplasia 6, 611-622.

Gutierrez-Barrera, A. M., Menter, D. G., Abbruzzese, J. L., and Reddy, S. A. (2007). Establishment of three-dimensional cultures of human pancreatic duct epithelial cells. Biochem Biophys Res Commun 358, 698-703.

Henshall, S. M., Afar, D. E., Hiller, J., Horvath, L. G., Quinn, D. I., Rasiah, K. K., Gish, K., Willhite, D., Kench, J. G., Gardiner-Garden, M., et al. (2003). Survival analysis of genome-wide gene expression profiles of prostate cancers identifies new prognostic targets of disease relapse. Cancer Res 63, 4196-4203.

Horvath, S., Zhang, B., Carlson, M., Lu, K. V., Zhu, S., Felciano, R. M., Laurance, M. F., Zhao, W., Qi, S., Chen, Z., et al. (2006). Analysis of oncogenic signaling networks in glioblastoma identifies ASPM as a molecular target. Proc Natl Acad Sci U S A 103, 17402-17407.

Hruban, R. H., and Fukushima, N. (2007). Pancreatic adenocarcinoma: update on the surgical pathology of carcinomas of ductal origin and PanINs. Mod Pathol 20 Suppl 1, S61-70.

Kenny, P. A., Lee, G. Y., Myers, C. A., Neve, R. M., Semeiks, J. R., Spellman, P. T., Lorenz, K., Lee, E. H., Barcellos-Hoff, M. H., Petersen, O. W., et al. (2007). The morphologies of breast cancer cell lines in three-dimensional assays correlate with their profiles of gene expression. Mol Oncol 1, 84-96.

Kolligs, F. T., Hu, G., Dang, C. V., and Fearon, E. R. (1999). Neoplastic transformation of RK3E by mutant beta-catenin requires deregulation of Tcf/Lef transcription but not activation of c-myc expression. Mol Cell Biol 19, 5696-5706.

Koprowski, H., Herlyn, M., Steplewski, Z., and Sears, H. F. (1981). Specific antigen in serum of patients with colon carcinoma. Science 212, 53-55.

Kouprina, N., Pavlicek, A., Collins, N. K., Nakano, M., Noskov, V. N., Ohzeki, J., Mochida, G. H., Risinger, J. I., Goldsmith, P., Gunsior, M., et al. (2005). The microcephaly ASPM gene is expressed in proliferating tissues and encodes for a mitotic spindle protein. Hum Mol Genet 14, 2155-2165.

Lee, G. Y., Kenny, P. A., Lee, E. H., and Bissell, M. J. (2007). Three-dimensional culture models of normal and malignant breast epithelial cells. Nat Methods 4, 359-365.

Li, C., Heidt, D. G., Dalerba, P., Burant, C. F., Zhang, L., Adsay, V., Wicha, M., Clarke, M. F., and Simeone, D. M. (2007). Identification of pancreatic cancer stem cells. Cancer Res 67, 1030-1037.

Lin, S. Y., Pan, H. W., Liu, S. H., Jeng, Y. M., Hu, F. C., Peng, S. Y., Lai, P. L., and Hsu, H. C. (2008). ASPM is a novel marker for vascular invasion, early recurrence, and poor prognosis of hepatocellular carcinoma. Clin Cancer Res 14, 4814-4820.

Liu, N., Furukawa, T., Kobari, M., and Tsao, M. S. (1998). Comparative phenotypic studies of duct epithelial cell lines derived from normal human pancreas and pancreatic carcinoma. Am J Pathol 153, 263-269.

Liu, R., Wang, X., Chen, G. Y., Dalerba, P., Gurney, A., Hoey, T., Sherlock, G., Lewicki, J., Shedden, K., and Clarke, M. F. (2007). The prognostic role of a gene signature from tumorigenic breast-cancer cells. N Engl J Med 356, 217-226.

Ma, X. J., Dahiya, S., Richardson, E., Erlander, M., and Sgroi, D. C. (2009). Gene expression profiling of the tumor microenvironment during breast cancer progression. Breast Cancer Res 11, R7.

Mondrinos, M. J., Koutzaki, S., Jiwanmall, E., Li, M., Dechadarevian, J. P., Lelkes, P. I., and Finck, C. M. (2006). Engineering three-dimensional pulmonary tissue constructs. Tissue Eng 12, 717-728.

Pasca di Magliano, M., Biankin, A. V., Heiser, P. W., Cano, D. A., Gutierrez, P. J., Deramaudt, T., Segara, D., Dawson, A. C., Kench, J. G., Henshall, S. M., et al. (2007). Common activation of canonical Wnt signaling in pancreatic adenocarcinoma. PLoS One 2, e1155.

Pawitan, Y., Bjohle, J., Amler, L., Borg, A. L., Egyhazi, S., Hall, P., Han, X., Holmberg, L., Huang, F., Klaar, S., et al. (2005). Gene expression profiling spares early breast cancer patients from adjuvant therapy: derived and validated in two population-based cohorts. Breast Cancer Res 7, R953-964.

Pencina, M. J., and D′Agostino, R. B. (2004). Overall C as a measure of discrimination in survival analysis: model specific population value and confidence interval estimation. Stat Med 23, 2109-2123.

Rakha, E. A., El-Sayed, M. E., Lee, A. H., Elston, C. W., Grainge, M. J., Hodi, Z., Blamey, R. W., and Ellis, I. O. (2008). Prognostic significance of Nottingham histologic grade in invasive breast carcinoma. J Clin Oncol 26, 3153-3158.

Ramaswamy, S., Ross, K. N., Lander, E. S., and Golub, T. R. (2003). A molecular signature of metastasis in primary solid tumors. Nat Genet 33, 49-54.

Richardson, A. L., Wang, Z. C., De Nicolo, A., Lu, X., Brown, M., Miron, A., Liao, X., Iglehart, J. D., Livingston, D. M., and Ganesan, S. (2006). X chromosomal abnormalities in basal-like human breast cancer. Cancer cell 9, 121-132.

Singh, D., Febbo, P. G., Ross, K., Jackson, D. G., Manola, J., Ladd, C., Tamayo, P., Renshaw, A. A., D'Amico, A. V., Richie, J. P., et al. (2002). Gene expression correlates of clinical prostate cancer behavior. Cancer cell 1, 203-209.

Sotiriou, C., Wirapati, P., Loi, S., Harris, A., Fox, S., Smeds, J., Nordgren, H., Farmer, P., Praz, V., Haibe-Kains, B., et al. (2006). Gene expression profiling in breast cancer: understanding the molecular basis of histologic grade to improve prognosis. Journal of the National Cancer Institute 98, 262-272.

Stamey, T. A., McNeal, J. E., Yemoto, C. M., Sigal, B. M., and Johnstone, I. M. (1999). Biological determinants of cancer progression in men with prostate cancer. JAMA 281, 1395-1400.

Stratford, J. K., Bentrem, D. J., Anderson, J. M., Fan, C., Volmar, K. A., Marron, J. S., Routh, E. D., Caskey, L. S., Samuel, J. C., Der, C. J., et al. (2010). A six-gene signature predicts survival of patients with localized pancreatic ductal adenocarcinoma. PLoS Med 7, e1000307.

van't Veer, L. J., Dai, H., van de Vijver, M. J., He, Y. D., Hart, A. A., Mao, M., Peterse, H. L., van der Kooy, K., Marton, M. J., Witteveen, A. T., et al. (2002). Gene expression profiling predicts clinical outcome of breast cancer. Nature 415, 530-536.

van de Vijver, M. J., He, Y. D., van't Veer, L. J., Dai, H., Hart, A. A., Voskuil, D. W., Schreiber, G. J., Peterse, J. L., Roberts, C., Marton, M. J., et al. (2002). A gene-expression signature as a predictor of survival in breast cancer. N Engl J Med 347, 1999-2009.

Varambally, S., Yu, J., Laxman, B., Rhodes, D. R., Mehra, R., Tomlins, S. A., Shah, R. B., Chandran, U., Monzon, F. A., Becich, M. J., et al. (2005). Integrative genomic and proteomic analysis of prostate cancer reveals signatures of metastatic progression. Cancer cell 8, 393-406.

Vermeulen, L., De Sousa, E. M. F., van der Heijden, M., Cameron, K., de Jong, J. H., Borovski, T., Tuynman, J. B., Todaro, M., Merz, C., Rodermond, H., et al. (2010). Wnt activity defines colon cancer stem cells and is regulated by the microenvironment. Nat Cell Biol 12, 468-476.

Wang, L., Heidt, D. G., Lee, C. J., Yang, H., Logsdon, C. D., Zhang, L., Fearon, E. R., Ljungman, M., and Simeone, D. M. (2009). Oncogenic function of ATDC in pancreatic cancer through Wnt pathway activation and beta-catenin stabilization. Cancer cell 15, 207-219.

Wang, Y., Klijn, J. G., Zhang, Y., Sieuwerts, A. M., Look, M. P., Yang, F., Talantov, D., Timmermans, M., Meijer-van Gelder, M. E., Yu, J., et al. (2005). Gene-expression profiles to predict distant metastasis of lymph-node-negative primary breast cancer. Lancet 365, 671-679.

Weaver, V. M., Petersen, O. W., Wang, F., Larabell, C. A., Briand, P., Damsky, C., and Bissell, M. J. (1997). Reversion of the malignant phenotype of human breast cells in three-dimensional culture and in vivo by integrin blocking antibodies. J Cell Biol 137, 231-245.

Webber, M. M., Bello, D., Kleinman, H. K., and Hoffman, M. P. (1997). Acinar differentiation by non-malignant immortalized human prostatic epithelial cells and its loss by malignant cells. Carcinogenesis 18, 1225-1231.

Wong, D. J., Liu, H., Ridky, T. W., Cassarino, D., Segal, E., and Chang, H. Y. (2008). Module map of stem cell genes guides creation of epithelial cancer stem cells. Cell Stem Cell 2, 333-344.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. The citation of any references herein is not an admission that such references are prior art to the present invention.

Unless otherwise indicated, all numbers expressing quantities of ingredients, reaction conditions, and so forth used in the specification, including claims, are to be understood as approximations and may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches. The recitation of series of numbers with differing amounts of significant digits in the specification is not to be construed as implying that numbers with fewer significant digits given have the same precision as numbers with more significant digits given.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.”

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method of predicting the clinical prognosis of a subject having a pancreatic cancer comprising: (a) obtaining a measurement of the transcript or protein expression levels of one or more marker genes in one or more tumor samples from the subject, wherein the marker genes are selected from ASPM, ATP9A, ACOX3, CDC45L, SLC40A1, AGR2, and those found in TABLE 2; and (c) comparing the expression levels of the markers genes in the tumor sample to one or a plurality of threshold reference levels.

2. The method of claim 1, further comprising the step of assigning the tumor a clinical prognosis group based on the comparison(s) in step (c).

3. The method of any of claim 1 or 2, further comprising administering a therapeutically effective amount of an agent for inhibiting ASPM.

4. The method of claim 1, wherein determining the transcript expression comprises polymerase chain reaction, northern blotting, RNase protection assay, or cDNA or oligonucleotide microarray analysis; and determining the protein expression comprises immunoblotting, immunohistochemistry, protein array, or two-dimensional protein electrophoresis and mass spectroscopy analysis.

5. The method of claim 1, wherein the clinical prognosis comprises (a) the time interval between the date of disease diagnosis or surgery and the date of disease recurrence or metastasis; (b) the time interval between the date of disease diagnosis or surgery and the date of death of the subject; or (c) changes in the number, size, or volume of one or a plurality of measurable tumor lesions.

6. The method of claim 1, wherein determining the threshold reference levels comprising: (a) obtaining samples of tumors from a large number of subjects with pancreatic cancer and whose clinical prognosis data are available; (b) determining the expression levels of said markers in said samples; (c) rank ordering in descending order said large number of subjects according to said expression levels of said samples or their combination; and (d) determining one or a plurality of threshold reference levels wherein said subjects whose tumors have expression levels of said markers above said threshold reference level(s) are predicted as having a higher or lower risk of poor clinical prognosis or disease progression than those with expression levels below said threshold reference level(s).

7. A method of predicting the clinical prognosis of a subject having a glandular cancer comprising: (a) obtaining one or more samples of a tumor from a subject with a glandular cancer; (b) determining transcript or protein expression level of ASPM; (c) comparing the expression levels of ASPM in said tumor sample to one or a plurality of threshold reference levels; and (d) assigning the tumor a clinical prognosis group based on the comparison(s) in (c).

8. A method of treating a glandular cancer in an individual, the method comprising inhibiting the expression and/or the activity of ASPM in said cancer.

9. The method of claim 8, wherein the glandular cancers are pancreatic cancer, breast cancer, and prostate cancer.

10. The method of claim 8, wherein the method comprises administering to said individual a nucleic acid complimentary to an ASPM mRNA, including an small interfering RNA, small hairpin RNA, microRNA, or antisense oligonucleotide.

11. The method of claim 8, wherein the method further comprises administering to said individual said nucleic acid complimentary to an ASPM mRNA that is sufficient to inhibit the ability of ASPM to increase the activity of Wnt signaling pathway.

12. The method of claim 11, wherein the activity of Wnt signaling pathway are measured by β-catenin levels or T-cell factor (TCF)/lymphoid enhancer-binding factor 1 (LEF1) activity.

13. The method of claim 8, wherein the method further comprises administering to said individual said nucleic acid complimentary to an ASPM mRNA that is sufficient to inhibit the ability of ASPM to promote or to maintain cancer stem cell populations or their tumor-initiating and/or metastasis-promoting capabilities

14. The method of claim 13, wherein the cancer stem cells can be defined by one or more markers, which comprise CD44, CD24, epithelial specific antigen (ESA), CD133, chemokine (C-X-C motif) receptor 4 (CXCR4), aldehyde dehydrogenase (ALDH) or any combination of the foregoing.

15. A kit for assaying ASPM levels for evaluating risk, presence, stage, or severity of pancreatic cancer or glandular cancers, wherein the kit comprises:
a reagent capable of detecting ASPM levels in a biological sample of a subject and a test substrate; and
optional instructions for contacting the reagent or substrate with a sample from the subject and instructions for evaluating the risk, predisposition, or prognosis for pancreatic cancer or glandular cancer in a subject, wherein increased ASPM levels indicate an increased risk, an increased predisposition, or a poor prognosis.
